(19) United States
(12) Patent Application Publication
(10) Pub. No.: US 2010/0144538 A1
(43) Pub. Date: Jun. 10, 2010

(54) GENEMAP OF THE HUMAN GENES ASSOCIATED WITH SCHIZOPHRENIA

(75) Inventors: Abdelmajid Belouchi, Beaconsfield (CA); John Verner Raelson, Hudson Heights (CA); Walter Edward Bradley, Montreal (CA); Bruno Paquin, Chateauguay (CA); Helene Fournier, Montreal (CA); Pascal Croteau, Laval (CA); Nouzha Paquin, Chateauguay (CA); Daniel Dubois, Laval (CA); Vanessa Bruat, Montreal (CA); Paul Van Eerdewegh, Carlisle, MA (US); Jonathan Segal, Efrat (IL); Randall David Little, Dorothee, CA (US); Tim Keith, Bedford, MA (US)

Correspondence Address: DOWELL & DOWELL P.C., 103 Oronoco St., Suite 220, Alexandria, VA, 22314, US

(73) Assignee: GENIZON BIOSCIENCES INC.

(21) Appl. No.: 12/449,899

(22) PCT Filed: Mar. 10, 2008
(86) PCT No.: PCT/US08/03125

§ 371 Date: Oct. 13, 2009

Related U.S. Application Data
(60) Provisional application No. 60/905,611, filed on Mar. 8, 2007.

(51) Int. Cl. C40B 20/00; G01N 33/68; G01N 33/48; C12Q 1/02; C12Q 1/68; G01N 33/53; G01N 33/50

(52) U.S. Cl. 506/2; 436/86; 436/94; 435/29; 435/6; 436/501; 204/403.01

(57) ABSTRACT

The present invention relates to the selection of a set of polymorphism markers for use in genome wide association studies based on linkage disequilibrium mapping. In particular, the invention relates to the fields of pharmacogenomics, diagnostics, patient therapy and the use of genetic haplotype information to predict an individual's susceptibility to SCHIZOPHRENIA disease and/or their response to a particular drug or drugs.

### PRIORITY

This application claims priority to U.S. Provisional Application No. 60/905,611, filed Mar. 8, 2007, which is hereby incorporated by reference in its entirety.

### FIELD OF THE INVENTION

The invention relates to the field of genomics and genetics, including genome analysis and the study of DNA variations. In particular, the invention relates to the fields of pharmacogenomics, diagnostics, patient therapy and the use of genetic haplotype information to predict an individual's susceptibility to SCHIZOPHRENIA disease and/or their response to a particular drug or drugs, so that drugs tailored to genetic differences of population groups may be developed and/or administered to the appropriate population.

The invention also relates to a GeneMap for SCHIZOPHRENIA disease, which links variations in DNA (including both genic and non-genic regions) to an individual's susceptibility to SCHIZOPHRENIA disease and/or response to a particular drug or drugs. The invention further relates to the genes disclosed in the GeneMap (see Tables 2-4), which is related to methods and reagents for detection of an individual's increased or decreased risk for SCHIZOPHRENIA disease and related sub-phenotypes, by identifying at least one polymorphism in one or a combination of the genes from the GeneMap. Also related are the candidate regions identified in Table 1, which are associated with SCHIZOPHRENIA disease. In addition, the invention further relates to nucleotide sequences of those genes including genomic DNA sequences, DNA sequences, single nucleotide polymorphisms (SNPs), other types of polymorphisms (insertions, deletions, microsatellites), alleles and haplotypes (see Sequence Listing and Tables 5-35).

The invention further relates to isolated nucleic acids comprising these nucleotide sequences and isolated polypeptides or peptides encoded thereby. Also related are expression vectors and host cells comprising the disclosed nucleic acids or fragments thereof, as well as antibodies that bind to the encoded polypeptides or peptides.

The present invention further relates to ligands that modulate the activity of the disclosed genes or gene products. In addition, the invention relates to diagnostics and therapeutics for SCHIZOPHRENIA disease, utilizing the disclosed nucleic acids, polymorphisms, chromosomal regions, GeneMaps, polypeptides or peptides, antibodies and/or ligands and small molecules that activate or repress relevant signaling events.

### BACKGROUND OF THE INVENTION

Schizophrenia is a severe psychiatric condition that affects approximately one percent of the population worldwide (Lewis et al., 2000). People with schizophrenia often experience both “positive” symptoms (e.g., delusions, hallucinations, paranoia, psychosis, disorganized thinking, and agitation) and “negative” symptoms (e.g., lack of drive or initiative, social withdrawal, apathy, impaired attention, cognitive impairements and emotional unresponsiveness).

There are an estimated 45 million people with schizophrenia in the world, with more than 33 million of them in developing countries. This disease places a heavy burden on the patient's family and relatives, both in terms of the direct and indirect costs involved, and the social stigma associated with the illness, sometimes over generations. Moreover, schizophrenia accounts for one fourth of all mental health costs and takes up one in three psychiatric hospital beds. Most schizophrenia patients are never able to work. The cost of schizophrenia to society is enormous. The most common cause of death among schizophrenic patients is suicide (in 10% of patients) which represents a 20 times higher risk than for the general population. Deaths from heart disease and from diseases of the respiratory and digestive system are also increased among schizophrenic patients.

Studies of the inheritance of schizophrenia have revealed that it is a multi-factorial disease characterized by multiple genetic susceptibility elements; each likely contributing a modest increase in risk (Karayiorgou et al., 1997).

Complex disorders such as schizophrenia are believed to involve several genes rather than single genes, as observed in rare disorders. This makes detection of any particular gene substantially more difficult than in a rare disorder, where a single gene mutation segregating according to a Mendelian inheritance pattern is the causative mutation. Any one of the multiple interacting gene mutations involved in the etiology of a complex and common disorder will impart a lower relative risk for the disorder than will the single gene mutation involved in a simple genetic disorder. Low relative risk alleles are more difficult to detect and, as a result, the success of positional cloning using linkage mapping that was achieved for simple genetic disorder genes has not been repeated for complex disorders.

Several approaches have been proposed to discover and characterize multiple genes in complex genetic disorders. These gene discovery methods can be subdivided into hypothesis-free disorder association studies and hypothesis-driven candidate gene or region studies. The candidate gene approach relies on the analysis of a gene in patients who have a disorder or a genetic disorder in which the gene is thought to play a role. This approach is limited in utility because it only provides for the investigation of genes with known functions. Although variant sequences of candidate genes may be identified using this approach, it is inherently limited by the fact that variant sequences in other genes that contribute to the phenotype will be necessarily missed when the technique is employed. A genome-wide scan (GWS) has been shown to be efficient in identifying schizophrenia susceptibility markers, such as the NRG1 gene on chromosome 8. In contrast to the candidate gene approach, a GWS searches throughout the genome without any a priori hypothesis and consequently can identify genes that are not obvious candidates for the complex genetic disorder as well as genes that are relevant candidates for the disorder. Furthermore, it can identify structurally important chromosomal regions a “that can influence the expression of specific, disorder-related genes.

Family-based linkage mapping methods were initially used for disorder locus identification. This technique locates genes based on the relatively limited number of genetic recombination events within the families used in the study, and results in large chromosomal regions containing hundreds of genes, any one of which could be the disorder-causing gene. Population-based, or linkage disequilibrium (LD) mapping is based on the premise that regions adjacent to a gene of interest are co-transmitted through the generations along with the gene. As a result, LD extends over shorter genetic regions than does linkage (Hewett et al., 2002), and can facilitate detection of genes with lower relative risk than family linkage mapping approaches. It also defines much smaller candidate regions which may contain only a few genes, making the identification of the actual disorder gene much easier.

It has been estimated that a GWS that uses a general population and case/control association (LD) analysis would require approximately 700,000 SNP markers (Carlson et al., 2003). The cost of a GWS at this marker density for a sufficient sample size for statistical power is economically prohibitive. The use of a founder population (genetic isolates), such as the French Canadian population of Quebec, is one solution to the problem with LD analysis. The French Canadian population in Quebec (Quebec Founder Population—QFP) provides one of the best resources in the world for gene discovery based on its high levels of genetic sharing and genetic homogeneity. By combining DNA collected from the QFP, high throughput genotyping capabilities and proprietary algorithms for genetic analysis, a comprehensive genome-wide association study is facilitated. The present invention relates specifically to a set of schizophrenia-related genes (GeneMap) and targets which present attractive points of therapeutic intervention for schizophrenia.

Current treatments do not address the root cause of the disease. Despite a preponderance of evidence showing inheritance of a risk for SCHIZOPHRENIA disease through epidemiological studies and genome wide linkage analyses, the genes affecting SCHIZOPHRENIA disease have yet to be discovered. There is a need in the art for identifying specific genes related to SCHIZOPHRENIA disease to enable the development of therapeutics that address the causes of the disease rather than relieving its symptoms.

The present invention relates specifically to a set of SCHIZOPHRENIA disease-causing genes (GeneMap) and targets which present attractive points of therapeutic intervention and diagnostics.

In view of the foregoing, identifying susceptibility genes associated with SCHIZOPHRENIA disease and their respective biochemical pathways will facilitate the identification of diagnostic markers as well as novel targets for improved therapeutics. It will also improve the quality of life for those afflicted by this disease and will reduce the economic costs of these afflictions at the individual and societal level. The identification of those genetic markers would provide the basis for novel genetic tests and eliminate or reduce the therapeutic methods currently used. The identification of those genetic markers will also provide the development of effective therapeutic intervention for the battery of laboratory, psychological and clinical evaluations typically required to diagnose SCHIZOPHRENIA. The present invention satisfies this need.

### DESCRIPTION OF THE FILES CONTAINED ON THE CD-R

The contents of the submission on compact discs submitted herewith are incorporated herein by reference in their entirety: A compact disc copy of the Sequence Listing (COPY 1) (filename: GENI 026 01WO SeqList.txt, date recorded: Mar. 10, 2008, file size: 37,722 kilobytes); a duplicate compact disc copy of the Sequence Listing (COPY 2) (filename: GENI 026 01WO SeqList.txt, date recorded: Mar. 10, 2008, file size: 37,722 kilobytes); a duplicate compact disc copy of the Sequence Listing (COPY 3) (filename: GENI 026 01WO SeqList.txt, date recorded: Mar. 10, 2008, file size: 37,722 kilobytes); a computer readable format copy of the Sequence Listing (CRF COPY) (filename: GENI 026 01WO SeqList.txt, date recorded: Mar. 10, 2008, file size: 37,722 kilobytes).

Three compact disc copies (COPY 1, COPY 2 and COPY 3) of Tables 1-38 are herewith submitted and are incorporated herein by reference in their entirety. Each compact disc contains a copy of the following files:

filename: Table1.txt, date recorded: Mar. 10, 2008, file size: 55 kilobytes;
filename: Table2.txt, date recorded: Mar. 10, 2008, file size: 426 kilobytes;
filename: Table3.txt, date recorded: Mar. 10, 2008, file size: 670 kilobytes;
filename: Table4.txt, date recorded: Mar. 10, 2008, file size: 2 kilobytes;
filename: Table5.1.txt, date recorded: Mar. 10, 2008, file size: 3 kilobytes;
filename: Table5.2.txt, date recorded: Mar. 10, 2008, file size: 3 kilobytes;
filename: Table6.1.txt, date recorded: Mar. 10, 2008, file size: 14 kilobytes;
filename: Table6.2.txt, date recorded: Mar. 10, 2008, file size: 99 kilobytes;
filename: Table7.1.txt, date recorded: Mar. 10, 2008, file size: 55 kilobytes;
filename: Table7.2.txt, date recorded: Mar. 10, 2008, file size: 178 kilobytes;
filename: Table8.1.txt, date recorded: Mar. 10, 2008, file size: 19 kilobytes;
filename: Table8.2.txt, date recorded: Mar. 10, 2008, file size: 49 kilobytes;
filename: Table9.1.txt, date recorded: Mar. 10, 2008, file size: 28 kilobytes;
filename: Table9.2.txt, date recorded: Mar. 10, 2008, file size: 27 kilobytes;
filename: Table9.3.txt, date recorded: Mar. 10, 2008, file size: 165 kilobytes;
filename: Table9.4.txt, date recorded: Mar. 10, 2008, file size: 164 kilobytes;
filename: Table10.1.txt, date recorded: Mar. 10, 2008, file size: 20 kilobytes;
filename: Table10.2.txt, date recorded: Mar. 10, 2008, file size: 24 kilobytes;
filename: Table11.1.txt, date recorded: Mar. 10, 2008, file size: 67 kilobytes;
filename: Table11.2.txt, date recorded: Mar. 10, 2008, file size: 336 kilobytes;
filename: Table12.1.txt, date recorded: Mar. 10, 2008, file size: 696 kilobytes;
filename: Table12.2.txt, date recorded: Mar. 10, 2008, file size: 1748 kilobytes;
filename: Table13.1.txt, date recorded: Mar. 10, 2008, file size: 55 kilobytes;
filename: Table13.2.txt, date recorded: Mar. 10, 2008, file size: 191 kilobytes;
filename: Table14.1.txt, date recorded: Mar. 10, 2008, file size: 12 kilobytes;
filename: Table14.2.txt, date recorded: Mar. 10, 2008, file size: 58 kilobytes;
filename: Table15.1.txt, date recorded: Mar. 10, 2008, file size: 66 kilobytes;
filename: Table15.2.txt date recorded: Mar. 10, 2008, file size: 359 kilobytes;
filename: Table16.1.txt, date recorded: Mar. 10, 2008, file size: 40 kilobytes;
filename: Table16.2.txt, date recorded: Mar. 10, 2008, file size: 38 kilobytes;
filename: Table16.3.txt, date recorded: Mar. 10, 2008, file size: 105 kilobytes;
filename: Table17.1.txt, date recorded: Mar. 10, 2008, file size: 21 kilobytes;
filename: Table17.2.txt, date recorded: Mar. 10, 2008, file size: 20 kilobytes;
filename: Table17.3.txt, date recorded: Mar. 10, 2008, file size: 44 kilobytes;
filename: Table18.1.txt, date recorded: Mar. 10, 2008, file size: 40 kilobytes;
filename: Table18.2.txt, date recorded: Mar. 10, 2008, file size: 39 kilobytes;
filename: Table18.3.txt, date recorded: Mar. 10, 2008, file size: 139 kilobytes;
filename: Table19.1.txt, date recorded: Mar. 10, 2008, file size: 25 kilobytes;
filename: Table19.2.txt, date recorded: Mar. 10, 2008, file size: 21 kilobytes;
filename: Table19.3.txt, date recorded: Mar. 10, 2008, file size: 11 kilobytes;
filename: Table20.1.txt, date recorded: Mar. 10, 2008, file size: 30 kilobytes;
filename: Table20.2.txt, date recorded: Mar. 10, 2008, file size: 28 kilobytes;
filename: Table20.3.txt, date recorded: Mar. 10, 2008, file size: 131 kilobytes;
filename: Table21.1.txt, date recorded: Mar. 10, 2008, file size: 32 kilobytes;
filename: Table21.2.txt, date recorded: Mar. 10, 2008, file size: 29 kilobytes;
filename: Table22.1.txt, date recorded: Mar. 10, 2008, file size: 194 kilobytes;
filename: Table22.2.txt, date recorded: Mar. 10, 2008, file size: 567 kilobytes;
filename: Table23.1.txt, date recorded: Mar. 10, 2008, file size: 55 kilobytes;
filename: Table23.2.txt, date recorded: Mar. 10, 2008, file size: 101 kilobytes;
filename: Table24.1.txt, date recorded: Mar. 10, 2008, file size: 230 kilobytes;
filename: Table24.2.txt, date recorded: Mar. 10, 2008, file size: 552 kilobytes;
filename: Table25.1.txt, date recorded: Mar. 10, 2008, file size: 8 kilobytes;
filename: Table25.2.txt, date recorded: Mar. 10, 2008, file size: 5 kilobytes;
filename: Table26.1.txt, date recorded: Mar. 10, 2008, file size: 36 kilobytes;
filename: Table26.2.txt, date recorded: Mar. 10, 2008, file size: 48 kilobytes;
filename: Table27.1.txt, date recorded: Mar. 10, 2008, file size: 170 kilobytes;
filename: Table27.2.txt, date recorded: Mar. 10, 2008, file size: 378 kilobytes;
filename: Table28.1.txt, date recorded: Mar. 10, 2008, file size: 6 kilobytes;
filename: Table28.2.txt, date recorded: Mar. 10, 2008, file size: 4 kilobytes;
filename: Table29.1.txt, date recorded: Mar. 10, 2008, file size: 10 kilobytes;
filename: Table29.2.txt, date recorded: Mar. 10, 2008, file size: 13 kilobytes;
filename: Table30.1.txt, date recorded: Mar. 10, 2008, file size: 35 kilobytes;
filename: Table30.2.txt, date recorded: Mar. 10, 2008, file size: 174 kilobytes;
filename: Table31.1.txt, date recorded: Mar. 10, 2008, file size: 36 kilobytes;
filename: Table31.2.txt, date recorded: Mar. 10, 2008, file size: 46 kilobytes;
filename: Table32.1.txt, date recorded: Mar. 10, 2008, file size: 61 kilobytes;
filename: Table32.2.txt, date recorded: Mar. 10, 2008, file size: 58 kilobytes;
filename: Table32.3.txt, date recorded: Mar. 10, 2008, file size: 241 kilobytes;
filename: Table33.1.txt, date recorded: Mar. 10, 2008, file size: 9 kilobytes;
filename: Table33.2.txt, date recorded: Mar. 10, 2008, file size: 47 kilobytes;
filename: Table34.1, date recorded: Mar. 10, 2008, file size: 7 kilobytes;
filename: Table34.2.txt, date recorded: Mar. 10, 2008, file size: 9 kilobytes;
filename: Table35.1.txt, date recorded: Mar. 10, 2008, file size: 27 kilobytes;
filename: Table35.2.txt, date recorded: Mar. 10, 2008, file size: 65 kilobytes;
filename: Table36.txt, date recorded: Mar. 10, 2008, file size: 15 kilobytes;
filename: Table37.txt, date recorded: Mar. 10, 2008, file size: 31 kilobytes; and
filename: Table38.txt, date recorded: Mar. 10, 2008, file size: 8 kilobytes.

LENGTHY TABLES
The patent application contains a lengthy table section. A copy of the table is available in electronic form from the USPTO web site (http://seqdata.uspto.gov/?pageRequest=docDetail&DocID=US20100144538A1). An electronic copy of the table will also be available from the USPTO upon request and payment of the fee set forth in 37 CFR 1.19(b)(3).

### TABLE DESCRIPTIONS

Table 1. List of schizophrenia disease candidate regions identified from the Genome Wide Scan association analyses. The first column denotes the region identifier. The second and third columns correspond to the chromosome and cytogenetic band, respectively. The fourth and fifth columns correspond to the chromosomal start and end coordinates of the NCBI genome assembly derived from build 36.
Table 2. List of candidate genes from the regions identified from the genome wide association analysis. The first column corresponds to the region identifier provided in Table 1. The second and third columns correspond to the chromosome and cytogenetic band, respectively. The fourth and fifth columns corresponds to the chromosomal start coordinates of the NCBI genome assembly derived from build 36 (B36) and the end coordinates (the start and end position relate to the +orientation of the NCBI assembly and don't necessarily correspond to the orientation of the gene). The sixth and seventh columns correspond to the official gene symbol and gene name, respectively, and were obtained from the NCBI Entrez Gene database. The eighth column corresponds to the NCBI Entrez Gene Identifier (GeneID). The ninth and tenth columns correspond to the Sequence IDs from nucleotide (cDNA) and protein entries in the Sequence Listing.
Table 3. List of candidate genes based on EST clustering from the regions identified from the various genome wide analyses. The first column corresponds to the region identifier provided in Table 1. The second column corresponds to the chromosome number. The third and fourth columns correspond to the chromosomal start and end coordinates of the NCBI genome assemblies derived from build 36 (B36). The fifth column corresponds to the ECGene Identifier, corresponding to the ECGene track of UCSC. These ECGene entries were determined by their overlap with the regions from Table 1, based on the start and end coordinates of both Region and ECGene identifiers. The sixth and seventh columns correspond to the Sequence IDs from nucleotide and protein entries in the Sequence Listing.
Table 4. List of micro RNA (miRNA) from the regions identified from the genome wide association analyses derived from build 36 (B36). To identify the miRNA from B36, these miRNA entries were determined by their overlap with the regions from Table 1, based on the start and end coordinates of both Region and miRNA identifiers. The first column corresponds to the region identifier provided in Table 1. The second column corresponds to the chromosome number. The third and fourth columns correspond to the chromosomal start and end coordinates of the NCBI genome assembly derived from build 36 (the start and end position relate to the + orientation of the NCBI assembly and do not necessarily correspond to the orientation of the miRNA). The fifth and sixth columns correspond to the miRNA accession and miRNA id, respectively, and were obtained from the miRBase database. The seventh column corresponds to the NCBI Entrez Gene Identifier (GeneID). The eighth column corresponds to the Sequence ID from nucleotide (RNA) in the Sequence Listing.
Table 5.1. Genome wide association study results in the Quebec Founder Population (QFP). SNP markers found to be associated with schizophrenia from the analysis of genome wide scan (GWS) data: CIAS1-1_cr1_not_w1. Columns include: Region ID; Chromosome; Build 36 location in base pairs (bp); rs#, dbSNP data base (NCBI) reference number; Sequence ID, unique numerical identifier for this patent application; Sequence, 21 by of sequence covering 10 base pair of unique sequence flanking either side of central polymorphic SNP; −log 10 P values for GWS, −log 10 of the P value for statistical significance from the GWS for single SNP markers (both T test and Permutation test p-values are displayed; see Example section) and for the most highly associated multi-marker haplotypes centered at the reference marker and defined by the sliding windows of specified sizes.
Table 5.2. List of significantly associated haplotypes based on the schizophrenia Disease GWS results using the Quebec Founder Population (QFP). Individual haplotypes with associated relative risks are presented in each row of the table; these values were extracted from the associated marker haplotype window with the most significant p value for each SNP in Table 5.1. The first column lists the region ID as presented in Table 1. The Haplotype column lists the specific nucleotides for the individual SNP alleles contributing to the haplotype reported. The Case and Control columns correspond to the numbers of cases and controls, respectively, containing the haplotype variant noted in the Haplotype column. The Total Case and Total Control columns list the total numbers of cases and controls for which genotype data was available for the haplotype in question. The RR column gives to the relative risk for each particular haplotype. The remainder of the columns lists the SeqIDs for the SNPs contributing to the haplotype and their relative location with respect to the central marker. The Central marker (0) column lists the SeqID for the central marker on which the haplotype is based. Flanking markers are identified by minus (−) or plus (+) signs to indicate the relative location of flanking SNPs.
Table 6.1. Genome wide association study results in the Quebec Founder Population (QFP). SNP markers found to be associated with schizophrenia from the analysis of genome wide scan (GWS) data: PTPRD-1_cr2-not. Columns include: Region ID; Chromosome; Build 36 location in base pairs (bp); rs#, dbSNP data base (NCBI) reference number; Sequence ID, unique numerical identifier for this patent application; Sequence, 21 by of sequence covering 10 base pair of unique sequence flanking either side of central polymorphic SNP; −log 10 P values for GWS, −log 10 of the P value for statistical significance from the GWS for single SNP markers (both T test and Permutation test p-values are displayed; see Example section) and for the most highly associated multi-marker haplotypes centered at the reference marker and defined by the sliding windows of specified sizes.
Table 6.2. List of significantly associated haplotypes based on the schizophrenia Disease GWS results using the Quebec Founder Population (QFP). Individual haplotypes with associated relative risks are presented in each row of the table; these values were extracted from the associated marker haplotype window with the most significant p value for each SNP in Table 6.1. The first column lists the region ID as presented in Table 1. The Haplotype column lists the specific nucleotides for the individual SNP alleles contributing to the haplotype reported. The Case and Control columns correspond to the numbers of cases and controls, respectively, containing the haplotype variant noted in the Haplotype column. The Total Case and Total Control columns list the total numbers of cases and controls for which genotype data was available for the haplotype in question. The RR column gives to the relative risk for each particular haplotype. The remainder of the columns lists the SeqIDs for the SNPs contributing to the haplotype and their relative location with respect to the central marker. The Central marker (0) column lists the SeqID for the central marker on which the haplotype is based. Flanking markers are identified by minus (−) or plus (+) signs to indicate the relative location of flanking SNPs.
Table 7.1. Genome wide association study results in the Quebec Founder Population (QFP). SNP markers found to be associated with schizoprenia from the analysis of genome wide scan (GWS) data: SPG3A-1_cp_not. Columns include: Region ID; Chromosome; Build 36 location in base pairs (bp); rs#, dbSNP data base (NCBI) reference number; Sequence ID, unique numerical identifier for this patent application; Sequence, 21 by of sequence covering 10 base pair of unique sequence flanking either side of central polymorphic SNP; −log 10 P values for GWS, −log 10 of the P value for statistical significance from the GWS for single SNP markers (both T test and Permutation test p-values are displayed; see Example section) and for the most highly associated multi-marker haplotypes centered at the reference marker and defined by the sliding windows of specified sizes.
Table 7.2. List of significantly associated haplotypes based on the schizophrenia Disease GWS results using the Quebec Founder Population (QFP). Individual haplotypes with associated relative risks are presented in each row of the table; these values were extracted from the associated marker haplotype window with the most significant p value for each SNP in Table 7.1. The first column lists the region ID as presented in Table 1. The Haplotype column lists the specific nucleotides for the individual SNP alleles contributing to the haplotype reported. The Case and Control columns correspond to the numbers of cases and controls, respectively, containing the haplotype variant noted in the Haplotype column. The Total Case and Total Control columns list the total numbers of cases and controls for which genotype data was available for the haplotype in question. The RR column gives to the relative risk for each particular haplotype. The remainder of the columns lists the SeqIDs for the SNPs contributing to the haplotype and their relative location with respect to the central marker. The Central marker (0) column lists the SeqID for the central marker on which the haplotype is based. Flanking markers are identified by minus (−) or plus (+) signs to indicate the relative location of flanking SNPs.
Table 8.1. Genome wide association study results in the Quebec Founder Population (QFP). SNP markers found to be associated with schizophrenia from the analysis of genome wide scan (GWS) data: SPG3A-1_cp_has. Columns include: Region ID; Chromosome; Build 36 location in base pairs (bp); rs#, dbSNP data base (NCBI) reference number; Sequence ID, unique numerical identifier for this patent application; Sequence, 21 by of sequence covering 10 base pair of unique sequence flanking either side of central polymorphic SNP; −log 10 P values for GWS, −log 10 of the P value for statistical significance from the GWS for single SNP markers (both T test and Permutation test p-values are displayed; see Example section) and for the most highly associated multi-marker haplotypes centered at the reference marker and defined by the sliding windows of specified sizes.
Table 8.2. List of significantly associated haplotypes based on the schizophrenia Disease GWS results using the Quebec Founder Population (QFP). Individual haplotypes with associated relative risks are presented in each row of the table; these values were extracted from the associated marker haplotype window with the most significant p value for each SNP in Table 8.1. The first column lists the region ID as presented in Table 1. The Haplotype column lists the specific nucleotides for the individual SNP alleles contributing to the haplotype reported. The Case and Control columns correspond to the numbers of cases and controls, respectively, containing the haplotype variant noted in the Haplotype column. The Total Case and Total Control columns list the total numbers of cases and controls for which genotype data was available for the haplotype in question. The RR column gives to the relative risk for each particular haplotype. The remainder of the columns lists the SeqIDs for the SNPs contributing to the haplotype and their relative location with respect to the central marker. The Central marker (0) column lists the SeqID for the central marker on which the haplotype is based. Flanking markers are identified by minus (−) or plus (+) signs to indicate the relative location of flanking SNPs.
Table 9.1. Genome wide association study results in the Quebec Founder Population (QFP). SNP markers found to be associated with schizophrenia from the analysis of genome wide scan (GWS) data: SPG3A-1-cr1_not (all results not to claim). Columns include: Region ID; Chromosome; Build 36 location in base pairs (bp); rs#, dbSNP data base (NCBI) reference number; Sequence ID, unique numerical identifier for this patent application; Sequence, 21 by of sequence covering 10 base pair of unique sequence flanking either side of central polymorphic SNP; −log 10 P values for GWS, −log 10 of the P value for statistical significance from the GWS for single SNP markers (both T test and Permutation test p-values are displayed; see Example section) and for the most highly associated multi-marker haplotypes centered at the reference marker and defined by the sliding windows of specified sizes.
Table 9.2. Genome wide association study results in the Quebec Founder Population (QFP). SNP markers found to be associated with schizophrenia from the analysis of genome wide scan (GWS) data: SPG3A-1-cr1_not (to claim). Columns include: Region ID; Chromosome; Build 36 location in base pairs (bp); rs#, dbSNP data base (NCBI) reference number; Sequence ID, unique numerical identifier for this patent application; Sequence, 21 by of sequence covering 10 base pair of unique sequence flanking either side of central polymorphic SNP; −log 10 P values for GWS, −log 10 of the P value for statistical significance from the GWS for single SNP markers (both T test and Permutation test p-values are displayed; see Example section) and for the most highly associated multi-marker haplotypes centered at the reference marker and defined by the sliding windows of specified sizes.
Table 9.3. List of significantly associated haplotypes based on the schizophrenia Disease GWS results using the Quebec Founder Population (QFP). Individual haplotypes with associated relative risks are presented in each row of the table; these values were extracted from the associated marker haplotype window with the most significant p value for each SNP in Table 9.1 (not to claim). The first column lists the region ID as presented in Table 1. The Haplotype column lists the specific nucleotides for the individual SNP alleles contributing to the haplotype reported. The Case and Control columns correspond to the numbers of cases and controls, respectively, containing the haplotype variant noted in the Haplotype column. The Total Case and Total Control columns list the total numbers of cases and controls for which genotype data was available for the haplotype in question. The RR column gives to the relative risk for each particular haplotype. The remainder of the columns lists the SeqIDs for the SNPs contributing to the haplotype and their relative location with respect to the central marker. The Central marker (0) column lists the SeqID for the central marker on which the haplotype is based. Flanking markers are identified by minus (−) or plus (+) signs to indicate the relative location of flanking SNPs.
Table 9.4. List of significantly associated haplotypes based on the schizophrenia Disease GWS results using the Quebec Founder Population (QFP). Individual haplotypes with associated relative risks are presented in each row of the table; these values were extracted from the associated marker haplotype window with the most significant p value for each SNP in Table 9.2 (to claim). The first column lists the region ID as presented in Table 1. The Haplotype column lists the specific nucleotides for the individual SNP alleles contributing to the haplotype reported. The Case and Control columns correspond to the numbers of cases and controls, respectively, containing the haplotype variant noted in the Haplotype column. The Total Case and Total Control columns list the total numbers of cases and controls for which genotype data was available for the haplotype in question. The RR column gives to the relative risk for each particular haplotype. The remainder of the columns lists the SeqIDs for the SNPs contributing to the haplotype and their relative location with respect to the central marker. The Central marker (0) column lists the SeqID for the central marker on which the haplotype is based. Flanking markers are identified by minus (−) or plus (+) signs to indicate the relative location of flanking SNPs.
Table 10.1. Genome wide association study results in the Quebec Founder Population (QFP). SNP markers found to be associated with schizophrenia from the analysis of genome wide scan (GWS) data: PAFAH1B1-1-cr_has. Columns include: Region ID; Chromosome; Build 36 location in base pairs (bp); rs#, dbSNP data base (NCBI) reference number; Sequence ID, unique numerical identifier for this patent application; Sequence, 21 by of sequence covering 10 base pair of unique sequence flanking either side of central polymorphic SNP; −log 10 P values for GWS, −log 10 of the P value for statistical significance from the GWS for single SNP markers.
Table 10.2. List of significantly associated haplotypes based on the schizophrenia Disease GWS results using the Quebec Founder Population (QFP). Individual haplotypes with associated relative risks are presented in each row of the table; these values were extracted from the associated marker haplotype window with the most significant p value for each SNP in Table 10.1. The first column lists the region ID as presented in Table 1. The Haplotype column lists the specific nucleotides for the individual SNP alleles contributing to the haplotype reported. The Case and Control columns correspond to the numbers of cases and controls, respectively, containing the haplotype variant noted in the Haplotype column. The Total Case and Total Control columns list the total numbers of cases and controls for which genotype data was available for the haplotype in question. The RR column gives to the relative risk for each particular haplotype. The remainder of the columns lists the SeqIDs for the SNPs contributing to the haplotype and their relative location with respect to the central marker. The Central marker (0) column lists the SeqID for the central marker on which the haplotype is based. Flanking markers are identified by minus (−) or plus (+) signs to indicate the relative location of flanking SNPs.
Table 11.1. Genome wide association study results in the Quebec Founder Population (QFP). SNP markers found to be associated with schizophrenia from the analysis of genome wide scan (GWS) data: PAFAH1B1-1-cr_not. Columns include: Region ID; Chromosome; Build 36 location in base pairs (bp); rs#, dbSNP data base (NCBI) reference number; Sequence ID, unique numerical identifier for this patent application; Sequence, 21 by of sequence covering 10 base pair of unique sequence flanking either side of central polymorphic SNP; −log 10 P values for GWS, −log 10 of the P value for statistical significance from the GWS for single SNP markers and for the most highly associated multi-marker haplotypes centered at the reference marker and defined by the sliding windows of specified sizes.
Table 11.2. List of significantly associated haplotypes based on the schizophrenia Disease GWS results using the Quebec Founder Population (QFP). Individual haplotypes with associated relative risks are presented in each row of the table; these values were extracted from the associated marker haplotype window with the most significant p value for each SNP in Table 11.1. The first column lists the region ID as presented in Table 1. The Haplotype column lists the specific nucleotides for the individual SNP alleles contributing to the haplotype reported. The Case and Control columns correspond to the numbers of cases and controls, respectively, containing the haplotype variant noted in the Haplotype column. The Total Case and Total Control columns list the total numbers of cases and controls for which genotype data was available for the haplotype in question. The RR column gives to the relative risk for each particular haplotype. The remainder of the columns lists the SeqIDs for the SNPs contributing to the haplotype and their relative location with respect to the central marker. The Central marker (0) column lists the SeqID for the central marker on which the haplotype is based. Flanking markers are identified by minus (−) or plus (+) signs to indicate the relative location of flanking SNPs.
Table 12.1. Genome wide association study results in the Quebec Founder Population (QFP). SNP markers found to be associated with schizophrenia from the analysis of genome wide scan (GWS) data: Full_sample. Columns include: Region ID; Chromosome; Build 36 location in base pairs (bp); rs#, dbSNP data base (NCBI) reference number; Sequence ID, unique numerical identifier for this patent application; Sequence, 21 by of sequence covering 10 base pair of unique sequence flanking either side of central polymorphic SNP; −log 10 P values for GWS, −log 10 of the P value for statistical significance from the GWS for single SNP markers (both T test and Permutation test p-values are displayed; see Example section) and for the most highly associated multi-marker haplotypes centered at the reference marker and defined by the sliding windows of specified sizes.
Table 12.2. List of significantly associated haplotypes based on the schizophrenia Disease GWS results using the Quebec Founder Population (QFP). Individual haplotypes with associated relative risks are presented in each row of the table; these values were extracted from the associated marker haplotype window with the most significant p value for each SNP in Table 12.1. The first column lists the region ID as presented in Table 1. The Haplotype column lists the specific nucleotides for the individual SNP alleles contributing to the haplotype reported. The Case and Control columns correspond to the numbers of cases and controls, respectively, containing the haplotype variant noted in the Haplotype column. The Total Case and Total Control columns list the total numbers of cases and controls for which genotype data was available for the haplotype in question. The RR column gives to the relative risk for each particular haplotype. The remainder of the columns lists the SeqIDs for the SNPs contributing to the haplotype and their relative location with respect to the central marker. The Central marker (0) column lists the SeqID for the central marker on which the haplotype is based. Flanking markers are identified by minus (−) or plus (+) signs to indicate the relative location of flanking SNPs.
Table 13.1. Genome wide association study results in the Quebec Founder Population (QFP). SNP markers found to be associated with schizophrenia from the analysis of genome wide scan (GWS) data: Paranoid. Columns include: Region ID; Chromosome; Build 36 location in base pairs (bp); rs#, dbSNP data base (NCBI) reference number; Sequence ID, unique numerical identifier for this patent application; Sequence, 21 by of sequence covering 10 base pair of unique sequence flanking either side of central polymorphic SNP; −log 10 P values for GWS, −log 10 of the P value for statistical significance from the GWS for single SNP markers (both T test and Permutation test p-values are displayed; see Example section) and for the most highly associated multi-marker haplotypes centered at the reference marker and defined by the sliding windows of specified sizes.
Table 13.2. List of significantly associated haplotypes based on the schizophrenia Disease GWS results using the Quebec Founder Population (QFP). Individual haplotypes with associated relative risks are presented in each row of the table; these values were extracted from the associated marker haplotype window with the most significant p value for each SNP in Table 13.1. The first column lists the region ID as presented in Table 1. The Haplotype column lists the specific nucleotides for the individual SNP alleles contributing to the haplotype reported. The Case and Control columns correspond to the numbers of cases and controls, respectively, containing the haplotype variant noted in the Haplotype column. The Total Case and Total Control columns list the total numbers of cases and controls for which genotype data was available for the haplotype in question. The RR column gives to the relative risk for each particular haplotype. The remainder of the columns lists the SeqIDs for the SNPs contributing to the haplotype and their relative location with respect to the central marker. The Central marker (0) column lists the SeqID for the central marker on which the haplotype is based. Flanking markers are identified by minus (−) or plus (+) signs to indicate the relative location of flanking SNPs.
Table 14.1. Genome wide association study results in the Quebec Founder Population (QFP). SNP markers found to be associated with schizophrenia from the analysis of genome wide scan (GWS) data: NRG1-1_cp1-has. Columns include: Region ID; Chromosome; Build 36 location in base pairs (bp); rs#, dbSNP data base (NCBI) reference number; Sequence ID, unique numerical identifier for this patent application; Sequence, 21 by of sequence covering 10 base pair of unique sequence flanking either side of central polymorphic SNP; −log 10 P values for GWS, −log 10 of the P value for statistical significance from the GWS for single SNP markers and for the most highly associated multi-marker haplotypes centered at the reference marker and defined by the sliding windows of specified sizes.
Table 14.2. List of significantly associated haplotypes based on the schizophrenia Disease GWS results using the Quebec Founder Population (QFP). Individual haplotypes with associated relative risks are presented in each row of the table; these values were extracted from the associated marker haplotype window with the most significant p value for each SNP in Table 14.1. The first column lists the region ID as presented in Table 1. The Haplotype column lists the specific nucleotides for the individual SNP alleles contributing to the haplotype reported. The Case and Control columns correspond to the numbers of cases and controls, respectively, containing the haplotype variant noted in the Haplotype column. The Total Case and Total Control columns list the total numbers of cases and controls for which genotype data was available for the haplotype in question. The RR column gives to the relative risk for each particular haplotype. The remainder of the columns lists the SeqIDs for the SNPs contributing to the haplotype and their relative location with respect to the central marker. The Central marker (0) column lists the SeqID for the central marker on which the haplotype is based. Flanking markers are identified by minus (−) or plus (+) signs to indicate the relative location of flanking SNPs.
Table 15.1. Genome wide association study results in the Quebec Founder Population (QFP). SNP markers found to be associated with schizophrenia from the analysis of genome wide scan (GWS) data: CIAS1-1_cr2_has. Columns include: Region ID; Chromosome; Build 36 location in base pairs (bp); rs#, dbSNP data base (NCBI) reference number; Sequence ID, unique numerical identifier for this patent application; Sequence, 21 by of sequence covering 10 base pair of unique sequence flanking either side of central polymorphic SNP; −log 10 P values for GWS, −log 10 of the P value for statistical significance from the GWS for single SNP markers and for the most highly associated multi-marker haplotypes centered at the reference marker and defined by the sliding windows of specified sizes.
Table 15.2. List of significantly associated haplotypes based on the schizophrenia Disease GWS results using the Quebec Founder Population (QFP). Individual haplotypes with associated relative risks are presented in each row of the table; these values were extracted from the associated marker haplotype window with the most significant p value for each SNP in Table 15.1. The first column lists the region ID as presented in Table 1. The Haplotype column lists the specific nucleotides for the individual SNP alleles contributing to the haplotype reported. The Case and Control columns correspond to the numbers of cases and controls, respectively, containing the haplotype variant noted in the Haplotype column. The Total Case and Total Control columns list the total numbers of cases and controls for which genotype data was available for the haplotype in question. The RR column gives to the relative risk for each particular haplotype. The remainder of the columns lists the SeqIDs for the SNPs contributing to the haplotype and their relative location with respect to the central marker. The Central marker (0) column lists the SeqID for the central marker on which the haplotype is based. Flanking markers are identified by minus (−) or plus (+) signs to indicate the relative location of flanking SNPs.
Table 16.1. Genome wide association study results in the Quebec Founder Population (QFP). SNP markers found to be associated with schizophrenia from the analysis of genome wide scan (GWS) data: NRG1-1_cp1-not (all results not to claim). Columns include: Region ID; Chromosome; Build 36 location in base pairs (bp); rs#, dbSNP data base (NCBI) reference number; Sequence ID, unique numerical identifier for this patent application; Sequence, 21 by of sequence covering 10 base pair of unique sequence flanking either side of central polymorphic SNP; −log 10 P values for GWS, −log 10 of the P value for statistical significance from the GWS for single SNP markers and for the most highly associated multi-marker haplotypes centered at the reference marker and defined by the sliding windows of specified sizes.
Table 16.2. Genome wide association study results in the Quebec Founder Population (QFP). SNP markers found to be associated with schizophrenia from the analysis of genome wide scan (GWS) data: NRG1-1_cp1-not (to claim). Columns include: Region ID; Chromosome; Build 36 location in base pairs (bp); rs#, dbSNP data base (NCBI) reference number; Sequence ID, unique numerical identifier for this patent application; Sequence, 21 by of sequence covering 10 base pair of unique sequence flanking either side of central polymorphic SNP; −log 10 P values for GWS, −log 10 of the P value for statistical significance from the GWS for single SNP markers and for the most highly associated multi-marker haplotypes centered at the reference marker and defined by the sliding windows of specified sizes.
Table 16.3. List of significantly associated haplotypes based on the schizophrenia Disease GWS results using the Quebec Founder Population (QFP). Individual haplotypes with associated relative risks are presented in each row of the table; these values were extracted from the associated marker haplotype window with the most significant p value for each SNP in Table 16.2. The first column lists the region ID as presented in Table 1. The Haplotype column lists the specific nucleotides for the individual SNP alleles contributing to the haplotype reported. The Case and Control columns correspond to the numbers of cases and controls, respectively, containing the haplotype variant noted in the Haplotype column. The Total Case and Total Control columns list the total numbers of cases and controls for which genotype data was available for the haplotype in question. The RR column gives to the relative risk for each particular haplotype. The remainder of the columns lists the SeqIDs for the SNPs contributing to the haplotype and their relative location with respect to the central marker. The Central marker (0) column lists the SeqID for the central marker on which the haplotype is based. Flanking markers are identified by minus (−) or plus (+) signs to indicate the relative location of flanking SNPs.
Table 17.1. Genome wide association study results in the Quebec Founder Population (QFP). SNP markers found to be associated with schizophrenia from the analysis of genome wide scan (GWS) data: NRG1-1_cp2-not (all results, not to claim). Columns include: Region ID; Chromosome; Build 36 location in base pairs (bp); rs#, dbSNP data base (NCBI) reference number; Sequence ID, unique numerical identifier for this patent application; Sequence, 21 by of sequence covering 10 base pair of unique sequence flanking either side of central polymorphic SNP; −log 10 P values for GWS, −log 10 of the P value for statistical significance from the GWS for single SNP markers and for the most highly associated multi-marker haplotypes centered at the reference marker and defined by the sliding windows of specified sizes.
Table 17.2. Genome wide association study results in the Quebec Founder Population (QFP). SNP markers found to be associated with schizophrenia from the analysis of genome wide scan (GWS) data: NRG1-1_cp2-not (to claim). Columns include: Region ID; Chromosome; Build 36 location in base pairs (bp); rs#, dbSNP data base (NCBI) reference number; Sequence ID, unique numerical identifier for this patent application; Sequence, 21 by of sequence covering 10 base pair of unique sequence flanking either side of central polymorphic SNP; −log 10 P values for GWS, −log 10 of the P value for statistical significance from the GWS for single SNP markers and for the most highly associated multi-marker haplotypes centered at the reference marker and defined by the sliding windows of specified sizes.
Table 17.3. List of significantly associated haplotypes based on the schizophrenia Disease GWS results using the Quebec Founder Population (QFP). Individual haplotypes with associated relative risks are presented in each row of the table; these values were extracted from the associated marker haplotype window with the most significant p value for each SNP in Table 17.2. The first column lists the region ID as presented in Table 1. The Haplotype column lists the specific nucleotides for the individual SNP alleles contributing to the haplotype reported. The Case and Control columns correspond to the numbers of cases and controls, respectively, containing the haplotype variant noted in the Haplotype column. The Total Case and Total Control columns list the total numbers of cases and controls for which genotype data was available for the haplotype in question. The RR column gives to the relative risk for each particular haplotype. The remainder of the columns lists the SeqIDs for the SNPs contributing to the haplotype and their relative location with respect to the central marker. The Central marker (0) column lists the SeqID for the central marker on which the haplotype is based. Flanking markers are identified by minus (−) or plus (+) signs to indicate the relative location of flanking SNPs.
Table 18.1. Genome wide association study results in the Quebec Founder Population (QFP). SNP markers found to be associated with schizophrenia from the analysis of genome wide scan (GWS) data: NRG1-1_cr1-has (all results, not to claim). Columns include: Region ID; Chromosome; Build 36 location in base pairs (bp); rs#, dbSNP data base (NCBI) reference number; Sequence ID, unique numerical identifier for this patent application; Sequence, 21 by of sequence covering 10 base pair of unique sequence flanking either side of central polymorphic SNP; −log 10 P values for GWS, −log 10 of the P value for statistical significance from the GWS for single SNP markers and for the most highly associated multi-marker haplotypes centered at the reference marker and defined by the sliding windows of specified sizes.
Table 18.2. Genome wide association study results in the Quebec Founder Population (QFP). SNP markers found to be associated with schizophrenia from the analysis of genome wide scan (GWS) data: NRG1-1_cr1-has (to claim). Columns include: Region ID; Chromosome; Build 36 location in base pairs (bp); rs#, dbSNP data base (NCBI) reference number; Sequence ID, unique numerical identifier for this patent application; Sequence, 21 by of sequence covering 10 base pair of unique sequence flanking either side of central polymorphic SNP; −log 10 P values for GWS, −log 10 of the P value for statistical significance from the GWS for single SNP markers and for the most highly associated multi-marker haplotypes centered at the reference marker and defined by the sliding windows of specified sizes.
Table 18.3. List of significantly associated haplotypes based on the schizophrenia Disease GWS results using the Quebec Founder Population (QFP). Individual haplotypes with associated relative risks are presented in each row of the table; these values were extracted from the associated marker haplotype window with the most significant p value for each SNP in Table 18.2. The first column lists the region ID as presented in Table 1. The Haplotype column lists the specific nucleotides for the individual SNP alleles contributing to the haplotype reported. The Case and Control columns correspond to the numbers of cases and controls, respectively, containing the haplotype variant noted in the Haplotype column. The Total Case and Total Control columns list the total numbers of cases and controls for which genotype data was available for the haplotype in question. The RR column gives to the relative risk for each particular haplotype. The remainder of the columns lists the SeqIDs for the SNPs contributing to the haplotype and their relative location with respect to the central marker. The Central marker (0) column lists the SeqID for the central marker on which the haplotype is based. Flanking markers are identified by minus (−) or plus (+) signs to indicate the relative location of flanking SNPs.
Table 19.1. Genome wide association study results in the Quebec Founder Population (QFP). SNP markers found to be associated with schizophrenia from the analysis of genome wide scan (GWS) data: NRG1-1_cr1-not (all results, not to claim. Columns include: Region ID; Chromosome; Build 36 location in base pairs (bp); rs#, dbSNP data base (NCBI) reference number; Sequence ID, unique numerical identifier for this patent application; Sequence, 21 by of sequence covering 10 base pair of unique sequence flanking either side of central polymorphic SNP; −log 10 P values for GWS, −log 10 of the P value for statistical significance from the GWS for single SNP markers and for the most highly associated multi-marker haplotypes centered at the reference marker and defined by the sliding windows of specified sizes.
Table 19.2. Genome wide association study results in the Quebec Founder Population (QFP). SNP markers found to be associated with schizophrenia from the analysis of genome wide scan (GWS) data: NRG1-1_cr1-not (to claim). Columns include: Region ID; Chromosome; Build 36 location in base pairs (bp); rs#, dbSNP data base (NCBI) reference number; Sequence ID, unique numerical identifier for this patent application; Sequence, 21 by of sequence covering 10 base pair of unique sequence flanking either side of central polymorphic SNP; −log 10 P values for GWS, −log 10 of the P value for statistical significance from the GWS for single SNP markers and for the most highly associated multi-marker haplotypes centered at the reference marker and defined by the sliding windows of specified sizes.
Table 19.3. List of significantly associated haplotypes based on the schizophrenia Disease GWS results using the Quebec Founder Population (QFP). Individual haplotypes with associated relative risks are presented in each row of the table; these values were extracted from the associated marker haplotype window with the most significant p value for each SNP in Table 19.2. The first column lists the region ID as presented in Table 1. The Haplotype column lists the specific nucleotides for the individual SNP alleles contributing to the haplotype reported. The Case and Control columns correspond to the numbers of cases and controls, respectively, containing the haplotype variant noted in the Haplotype column. The Total Case and Total Control columns list the total numbers of cases and controls for which genotype data was available for the haplotype in question. The RR column gives to the relative risk for each particular haplotype. The remainder of the columns lists the SeqIDs for the SNPs contributing to the haplotype and their relative location with respect to the central marker. The Central marker (0) column lists the SeqID for the central marker on which the haplotype is based. Flanking markers are identified by minus (−) or plus (+) signs to indicate the relative location of flanking SNPs.
Table 20.1. Genome wide association study results in the Quebec Founder Population (QFP). SNP markers found to be associated with schizophrenia from the analysis of genome wide scan (GWS) data: NRG1-1_cr2-has (all results, not to claim). Columns include: Region ID; Chromosome; Build 36 location in base pairs (bp); rs#, dbSNP data base (NCBI) reference number; Sequence ID, unique numerical identifier for this patent application; Sequence, 21 by of sequence covering 10 base pair of unique sequence flanking either side of central polymorphic SNP; −log 10 P values for GWS, −log 10 of the P value for statistical significance from the GWS for single SNP markers and for the most highly associated multi-marker haplotypes centered at the reference marker and defined by the sliding windows of specified sizes.
Table 20.2. Genome wide association study results in the Quebec Founder Population (QFP). SNP markers found to be associated with schizophrenia from the analysis of genome wide scan (GWS) data: NRG1-1_cr2-has (to claim). Columns include: Region ID; Chromosome; Build 36 location in base pairs (bp); rs#, dbSNP data base (NCBI) reference number; Sequence ID, unique numerical identifier for this patent application; Sequence, 21 by of sequence covering 10 base pair of unique sequence flanking either side of central polymorphic SNP; −log 10 P values for GWS, −log 10 of the P value for statistical significance from the GWS for single SNP markers and for the most highly associated multi-marker haplotypes centered at the reference marker and defined by the sliding windows of specified sizes.
Table 20.3. List of significantly associated haplotypes based on the schizophrenia Disease GWS results using the Quebec Founder Population (QFP). Individual haplotypes with associated relative risks are presented in each row of the table; these values were extracted from the associated marker haplotype window with the most significant p value for each SNP in Table 20.2. The first column lists the region ID as presented in Table 1. The Haplotype column lists the specific nucleotides for the individual SNP alleles contributing to the haplotype reported. The Case and Control columns correspond to the numbers of cases and controls, respectively, containing the haplotype variant noted in the Haplotype column. The Total Case and Total Control columns list the total numbers of cases and controls for which genotype data was available for the haplotype in question. The RR column gives to the relative risk for each particular haplotype. The remainder of the columns lists the SeqIDs for the SNPs contributing to the haplotype and their relative location with respect to the central marker. The Central marker (0) column lists the SeqID for the central marker on which the haplotype is based. Flanking markers are identified by minus (−) or plus (+) signs to indicate the relative location of flanking SNPs.
Table 21.1. Genome wide association study results in the Quebec Founder Population (QFP). SNP markers found to be associated with schizophrenia from the analysis of genome wide scan (GWS) data: NRG1-1_cr2-not. Columns include: Region ID; Chromosome; Build 36 location in base pairs (bp); rs#, dbSNP data base (NCBI) reference number; Sequence ID, unique numerical identifier for this patent application; Sequence, 21 by of sequence covering 10 base pair of unique sequence flanking either side of central polymorphic SNP; −log 10 P values for GWS, −log 10 of the P value for statistical significance from the GWS for single SNP markers and for the most highly associated multi-marker haplotypes centered at the reference marker and defined by the sliding windows of specified sizes.
Table 21.2. List of significantly associated haplotypes based on the schizophrenia Disease GWS results using the Quebec Founder Population (QFP). Individual haplotypes with associated relative risks are presented in each row of the table; these values were extracted from the associated marker haplotype window with the most significant p value for each SNP in Table 21.1. The first column lists the region ID as presented in Table 1. The Haplotype column lists the specific nucleotides for the individual SNP alleles contributing to the haplotype reported. The Case and Control columns correspond to the numbers of cases and controls, respectively, containing the haplotype variant noted in the Haplotype column. The Total Case and Total Control columns list the total numbers of cases and controls for which genotype data was available for the haplotype in question. The RR column gives to the relative risk for each particular haplotype. The remainder of the columns lists the SeqIDs for the SNPs contributing to the haplotype and their relative location with respect to the central marker. The Central marker (0) column lists the SeqID for the central marker on which the haplotype is based. Flanking markers are identified by minus (−) or plus (+) signs to indicate the relative location of flanking SNPs.
Table 22.1. Genome wide association study results in the Quebec Founder Population (QFP). SNP markers found to be associated with schizophrenia from the analysis of genome wide scan (GWS) data: Female Affected. Columns include: Region ID; Chromosome; Build 36 location in base pairs (bp); rs#, dbSNP data base (NCBI) reference number; Sequence ID, unique numerical identifier for this patent application; Sequence, 21 by of sequence covering 10 base pair of unique sequence flanking either side of central polymorphic SNP; −log 10 P values for GWS, −log 10 of the P value for statistical significance from the GWS for single SNP markers (both T test and Permutation test p-values are displayed; see Example section) and for the most highly associated multi-marker haplotypes centered at the reference marker and defined by the sliding windows of specified sizes.
Table 22.2 List of significantly associated haplotypes based on the schizophrenia Disease GWS results using the Quebec Founder Population (QFP). Individual haplotypes with associated relative risks are presented in each row of the table; these values were extracted from the associated marker haplotype window with the most significant p value for each SNP in Table 22.1. The first column lists the region ID as presented in Table 1. The Haplotype column lists the specific nucleotides for the individual SNP alleles contributing to the haplotype reported. The Case and Control columns correspond to the numbers of cases and controls, respectively, containing the haplotype variant noted in the Haplotype column. The Total Case and Total Control columns list the total numbers of cases and controls for which genotype data was available for the haplotype in question. The RR column gives to the relative risk for each particular haplotype. The remainder of the columns lists the SeqIDs for the SNPs contributing to the haplotype and their relative location with respect to the central marker. The Central marker (0) column lists the SeqID for the central marker on which the haplotype is based. Flanking markers are identified by minus (−) or plus (+) signs to indicate the relative location of flanking SNPs.
Table 23.1. Genome wide association study results in the Quebec Founder Population (QFP). SNP markers found to be associated with schizophrenia from the analysis of genome wide scan (GWS) data: Female_less_than_{—}25. Columns include: Region ID; Chromosome; Build 36 location in base pairs (bp); rs#, dbSNP data base (NCBI) reference number; Sequence ID, unique numerical identifier for this patent application; Sequence, 21 by of sequence covering 10 base pair of unique sequence flanking either side of central polymorphic SNP; −log 10 P values for GWS, −log 10 of the P value for statistical significance from the GWS for single SNP markers (both T test and Permutation test p-values are displayed; see Example section) and for the most highly associated multi-marker haplotypes centered at the reference marker and defined by the sliding windows of specified sizes.
Table 23.2. List of significantly associated haplotypes based on the schizophrenia Disease GWS results using the Quebec Founder Population (QFP). Individual haplotypes with associated relative risks are presented in each row of the table; these values were extracted from the associated marker haplotype window with the most significant p value for each SNP in Table 23.1. The first column lists the region ID as presented in Table 1. The Haplotype column lists the specific nucleotides for the individual SNP alleles contributing to the haplotype reported. The Case and Control columns correspond to the numbers of cases and controls, respectively, containing the haplotype variant noted in the Haplotype column. The Total Case and Total Control columns list the total numbers of cases and controls for which genotype data was available for the haplotype in question. The RR column gives to the relative risk for each particular haplotype. The remainder of the columns lists the SeqIDs for the SNPs contributing to the haplotype and their relative location with respect to the central marker. The Central marker (0) column lists the SeqID for the central marker on which the haplotype is based. Flanking markers are identified by minus (−) or plus (+) signs to indicate the relative location of flanking SNPs.
Table 24.1. Genome wide association study results in the Quebec Founder Population (QFP). SNP markers found to be associated with schizophrenia from the analysis of genome wide scan (GWS) data: Female_more_than_{—}25. Columns include: Region ID; Chromosome; Build 36 location in base pairs (bp); rs#, dbSNP data base (NCBI) reference number; Sequence ID, unique numerical identifier for this patent application; Sequence, 21 by of sequence covering 10 base pair of unique sequence flanking either side of central polymorphic SNP; −log 10 P values for GWS, −log 10 of the P value for statistical significance from the GWS for single SNP markers (both T test and Permutation test p-values are displayed; see Example section) and for the most highly associated multi-marker haplotypes centered at the reference marker and defined by the sliding windows of specified sizes.
Table 24.2. List of significantly associated haplotypes based on the schizophrenia Disease GWS results using the Quebec Founder Population (QFP). Individual haplotypes with associated relative risks are presented in each row of the table; these values were extracted from the associated marker haplotype window with the most significant p value for each SNP in Table 24.1. The first column lists the region ID as presented in Table 1. The Haplotype column lists the specific nucleotides for the individual SNP alleles contributing to the haplotype reported. The Case and Control columns correspond to the numbers of cases and controls, respectively, containing the haplotype variant noted in the Haplotype column. The Total Case and Total Control columns list the total numbers of cases and controls for which genotype data was available for the haplotype in question. The RR column gives to the relative risk for each particular haplotype. The remainder of the columns lists the SeqIDs for the SNPs contributing to the haplotype and their relative location with respect to the central marker. The Central marker (0) column lists the SeqID for the central marker on which the haplotype is based. Flanking markers are identified by minus (−) or plus (+) signs to indicate the relative location of flanking SNPs.
Table 25.1. Genome wide association study results in the Quebec Founder Population (QFP). SNP markers found to be associated with schizophrenia from the analysis of genome wide scan (GWS) data: Male Affected. Columns include: Region ID; Chromosome; Build 36 location in base pairs (bp); rs#, dbSNP data base (NCBI) reference number; Sequence ID, unique numerical identifier for this patent application; Sequence, 21 by of sequence covering 10 base pair of unique sequence flanking either side of central polymorphic SNP; −log 10 P values for GWS, −log 10 of the P value for statistical significance from the GWS for single SNP markers (both T test and Permutation test p-values are displayed; see Example section) and for the most highly associated multi-marker haplotypes centered at the reference marker and defined by the sliding windows of specified sizes.
Table 25.2. List of significantly associated haplotypes based on the schizophrenia Disease GWS results using the Quebec Founder Population (QFP). Individual haplotypes with associated relative risks are presented in each row of the table; these values were extracted from the associated marker haplotype window with the most significant p value for each SNP in Table 25.1. The first column lists the region ID as presented in Table 1. The Haplotype column lists the specific nucleotides for the individual SNP alleles contributing to the haplotype reported. The Case and Control columns correspond to the numbers of cases and controls, respectively, containing the haplotype variant noted in the Haplotype column. The Total Case and Total Control columns list the total numbers of cases and controls for which genotype data was available for the haplotype in question. The RR column gives to the relative risk for each particular haplotype. The remainder of the columns lists the SeqIDs for the SNPs contributing to the haplotype and their relative location with respect to the central marker. The Central marker (0) column lists the SeqID for the central marker on which the haplotype is based. Flanking markers are identified by minus (−) or plus (+) signs to indicate the relative location of flanking SNPs.
Table 26.1. Genome wide association study results in the Quebec Founder Population (QFP). SNP markers found to be associated with schizophrenia from the analysis of genome wide scan (GWS) data: WNT7A-1-cr1_has_w1. Columns include: Region ID; Chromosome; Build 36 location in base pairs (bp); rs#, dbSNP data base (NCBI) reference number; Sequence ID, unique numerical identifier for this patent application; Sequence, 21 by of sequence covering 10 base pair of unique sequence flanking either side of central polymorphic SNP; −log 10 P values for GWS, −log 10 of the P value for statistical significance from the GWS for single SNP markers.
Table 26.2. List of significantly associated haplotypes based on the schizophrenia Disease GWS results using the Quebec Founder Population (QFP). Individual haplotypes with associated relative risks are presented in each row of the table; these values were extracted from the associated marker haplotype window with the most significant p value for each SNP in Table 26.1. The first column lists the region ID as presented in Table 1. The Haplotype column lists the specific nucleotides for the individual SNP alleles contributing to the haplotype reported. The Case and Control columns correspond to the numbers of cases and controls, respectively, containing the haplotype variant noted in the Haplotype column. The Total Case and Total Control columns list the total numbers of cases and controls for which genotype data was available for the haplotype in question. The RR column gives to the relative risk for each particular haplotype. The remainder of the columns lists the SeqIDs for the SNPs contributing to the haplotype and their relative location with respect to the central marker. The Central marker (0) column lists the SeqID for the central marker on which the haplotype is based. Flanking markers are identified by minus (−) or plus (+) signs to indicate the relative location of flanking SNPs.
Table 27.1. Genome wide association study results in the Quebec Founder Population (QFP). SNP markers found to be associated with schizophrenia from the analysis of genome wide scan (GWS) data: Male less than 20. Columns include: Region ID; Chromosome; Build 36 location in base pairs (bp); rs#, dbSNP data base (NCBI) reference number; Sequence ID, unique numerical identifier for this patent application; Sequence, 21 by of sequence covering 10 base pair of unique sequence flanking either side of central polymorphic SNP; −log 10 P values for GWS, −log 10 of the P value for statistical significance from the GWS for single SNP markers (both T test and Permutation test p-values are displayed; see Example section) and for the most highly associated multi-marker haplotypes centered at the reference marker and defined by the sliding windows of specified sizes.
Table 27.2. List of significantly associated haplotypes based on the schizophrenia Disease GWS results using the Quebec Founder Population (QFP). Individual haplotypes with associated relative risks are presented in each row of the table; these values were extracted from the associated marker haplotype window with the most significant p value for each SNP in Table 27.1. The first column lists the region ID as presented in Table 1. The Haplotype column lists the specific nucleotides for the individual SNP alleles contributing to the haplotype reported. The Case and Control columns correspond to the numbers of cases and controls, respectively, containing the haplotype variant noted in the Haplotype column. The Total Case and Total Control columns list the total numbers of cases and controls for which genotype data was available for the haplotype in question. The RR column gives to the relative risk for each particular haplotype. The remainder of the columns lists the SeqIDs for the SNPs contributing to the haplotype and their relative location with respect to the central marker. The Central marker (0) column lists the SeqID for the central marker on which the haplotype is based. Flanking markers are identified by minus (−) or plus (+) signs to indicate the relative location of flanking SNPs.
Table 28.1 Male more than 20. Genome wide association study results in the Quebec Founder Population (QFP). SNP markers found to be associated with schizophrenia from the analysis of genome wide scan (GWS) data: Male more than 20. Columns include: Region ID; Chromosome; Build 36 location in base pairs (bp); rs#, dbSNP data base (NCBI) reference number; Sequence ID, unique numerical identifier for this patent application; Sequence, 21 by of sequence covering 10 base pair of unique sequence flanking either side of central polymorphic SNP; −log 10 P values for GWS, −log 10 of the P value for statistical significance from the GWS for single SNP markers (both T test and Permutation test p-values are displayed; see Example section) and for the most highly associated multi-marker haplotypes centered at the reference marker and defined by the sliding windows of specified sizes.
Table 28.2. List of significantly associated haplotypes based on the schizophrenia Disease GWS results using the Quebec Founder Population (QFP). Individual haplotypes with associated relative risks are presented in each row of the table; these values were extracted from the associated marker haplotype window with the most significant p value for each SNP in Table 28.1. The first column lists the region ID as presented in Table 1. The Haplotype column lists the specific nucleotides for the individual SNP alleles contributing to the haplotype reported. The Case and Control columns correspond to the numbers of cases and controls, respectively, containing the haplotype variant noted in the Haplotype column. The Total Case and Total Control columns list the total numbers of cases and controls for which genotype data was available for the haplotype in question. The RR column gives to the relative risk for each particular haplotype. The remainder of the columns lists the SeqIDs for the SNPs contributing to the haplotype and their relative location with respect to the central marker. The Central marker (0) column lists the SeqID for the central marker on which the haplotype is based. Flanking markers are identified by minus (−) or plus (+) signs to indicate the relative location of flanking SNPs.
Table 29.1. Genome wide association study results in the Quebec Founder Population (QFP). SNP markers found to be associated with schizophrenia from the analysis of genome wide scan (GWS) data: CIAS1-1-cr2_not. Columns include: Region ID; Chromosome; Build 36 location in base pairs (bp); rs#, dbSNP data base (NCBI) reference number; Sequence ID, unique numerical identifier for this patent application; Sequence, 21 by of sequence covering 10 base pair of unique sequence flanking either side of central polymorphic SNP; −log 10 P values for GWS, −log 10 of the P value for statistical significance from the GWS for single SNP markers.
Table 29.2. List of significantly associated haplotypes based on the schizophrenia Disease GWS results using the Quebec Founder Population (QFP). Individual haplotypes with associated relative risks are presented in each row of the table; these values were extracted from the associated marker haplotype window with the most significant p value for each SNP in Table 29.1. The first column lists the region ID as presented in Table 1. The Haplotype column lists the specific nucleotides for the individual SNP alleles contributing to the haplotype reported. The Case and Control columns correspond to the numbers of cases and controls, respectively, containing the haplotype variant noted in the Haplotype column. The Total Case and Total Control columns list the total numbers of cases and controls for which genotype data was available for the haplotype in question. The RR column gives to the relative risk for each particular haplotype. The remainder of the columns lists the SeqIDs for the SNPs contributing to the haplotype and their relative location with respect to the central marker. The Central marker (0) column lists the SeqID for the central marker on which the haplotype is based. Flanking markers are identified by minus (−) or plus (+) signs to indicate the relative location of flanking SNPs.
Table 30.1. Genome wide association study results in the Quebec Founder Population (QFP). SNP markers found to be associated with schizophrenia from the analysis of genome wide scan (GWS) data: WNT7A-1-cr1_not. Columns include: Region ID; Chromosome; Build 36 location in base pairs (bp); rs#, dbSNP data base (NCBI) reference number; Sequence ID, unique numerical identifier for this patent application; Sequence, 21 by of sequence covering 10 base pair of unique sequence flanking either side of central polymorphic SNP; −log 10 P values for GWS, −log 10 of the P value for statistical significance from the GWS for single SNP markers and for the most highly associated multi-marker haplotypes centered at the reference marker and defined by the sliding windows of specified sizes.
Table 30.2. List of significantly associated haplotypes based on the schizophrenia Disease GWS results using the Quebec Founder Population (QFP). Individual haplotypes with associated relative risks are presented in each row of the table; these values were extracted from the associated marker haplotype window with the most significant p value for each SNP in Table 30.1. The first column lists the region ID as presented in Table 1. The Haplotype column lists the specific nucleotides for the individual SNP alleles contributing to the haplotype reported. The Case and Control columns correspond to the numbers of cases and controls, respectively, containing the haplotype variant noted in the Haplotype column. The Total Case and Total Control columns list the total numbers of cases and controls for which genotype data was available for the haplotype in question. The RR column gives to the relative risk for each particular haplotype. The remainder of the columns lists the SeqIDs for the SNPs contributing to the haplotype and their relative location with respect to the central marker. The Central marker (0) column lists the SeqID for the central marker on which the haplotype is based. Flanking markers are identified by minus (−) or plus (+) signs to indicate the relative location of flanking SNPs.
Table 31.1. Genome wide association study results in the Quebec Founder Population (QFP). SNP markers found to be associated with schizophrenia from the analysis of genome wide scan (GWS) data: PTPRD-1_cp_not. Columns include: Region ID; Chromosome; Build 36 location in base pairs (bp); rs#, dbSNP data base (NCBI) reference number; Sequence ID, unique numerical identifier for this patent application; Sequence, 21 by of sequence covering 10 base pair of unique sequence flanking either side of central polymorphic SNP; −log 10 P values for GWS, −log 10 of the P value for statistical significance from the GWS for single SNP markers.
Table 31.2. List of significantly associated haplotypes based on the schizophrenia Disease GWS results using the Quebec Founder Population (QFP). Individual haplotypes with associated relative risks are presented in each row of the table; these values were extracted from the associated marker haplotype window with the most significant p value for each SNP in Table 31.1. The first column lists the region ID as presented in Table 1. The Haplotype column lists the specific nucleotides for the individual SNP alleles contributing to the haplotype reported. The Case and Control columns correspond to the numbers of cases and controls, respectively, containing the haplotype variant noted in the Haplotype column. The Total Case and Total Control columns list the total numbers of cases and controls for which genotype data was available for the haplotype in question. The RR column gives to the relative risk for each particular haplotype. The remainder of the columns lists the SeqIDs for the SNPs contributing to the haplotype and their relative location with respect to the central marker. The Central marker (0) column lists the SeqID for the central marker on which the haplotype is based. Flanking markers are identified by minus (−) or plus (+) signs to indicate the relative location of flanking SNPs.
Table 32.1. Genome wide association study results in the Quebec Founder Population (QFP). SNP markers found to be associated with schizophrenia from the analysis of genome wide scan (GWS) data: PTPRD-1_cp-has (all results, not to claim). Columns include: Region ID; Chromosome; Build 36 location in base pairs (bp); rs#, dbSNP data base (NCBI) reference number; Sequence ID, unique numerical identifier for this patent application; Sequence, 21 by of sequence covering 10 base pair of unique sequence flanking either side of central polymorphic SNP; −log 10 P values for GWS, −log 10 of the P value for statistical significance from the GWS for single SNP markers and for the most highly associated multi-marker haplotypes centered at the reference marker and defined by the sliding windows of specified sizes.
Table 32.2. Genome wide association study results in the Quebec Founder Population (QFP). SNP markers found to be associated with schizophrenia from the analysis of genome wide scan (GWS) data: PTPRD-1_cp-has (to claim). Columns include: Region ID; Chromosome; Build 36 location in base pairs (bp); rs#, dbSNP data base (NCBI) reference number; Sequence ID, unique numerical identifier for this patent application; Sequence, 21 by of sequence covering 10 base pair of unique sequence flanking either side of central polymorphic SNP; −log 10 P values for GWS, −log 10 of the P value for statistical significance from the GWS for single SNP markers and for the most highly associated multi-marker haplotypes centered at the reference marker and defined by the sliding windows of specified sizes.
Table 32.3. List of significantly associated haplotypes based on the schizophrenia Disease GWS results using the Quebec Founder Population (QFP). Individual haplotypes with associated relative risks are presented in each row of the table; these values were extracted from the associated marker haplotype window with the most significant p value for each SNP in Table 32.2. The first column lists the region ID as presented in Table 1. The Haplotype column lists the specific nucleotides for the individual SNP alleles contributing to the haplotype reported. The Case and Control columns correspond to the numbers of cases and controls, respectively, containing the haplotype variant noted in the Haplotype column. The Total Case and Total Control columns list the total numbers of cases and controls for which genotype data was available for the haplotype in question. The RR column gives to the relative risk for each particular haplotype. The remainder of the columns lists the SeqIDs for the SNPs contributing to the haplotype and their relative location with respect to the central marker. The Central marker (0) column lists the SeqID for the central marker on which the haplotype is based. Flanking markers are identified by minus (−) or plus (+) signs to indicate the relative location of flanking SNPs.
Table 33.1. Genome wide association study results in the Quebec Founder Population (QFP). SNP markers found to be associated with schizophrenia from the analysis of genome wide scan (GWS) data: PTPRD-1_cr1_not. Columns include: Region ID; Chromosome; Build 36 location in base pairs (bp); rs#, dbSNP data base (NCBI) reference number; Sequence ID, unique numerical identifier for this patent application; Sequence, 21 by of sequence covering 10 base pair of unique sequence flanking either side of central polymorphic SNP; −log 10 P values for GWS, −log 10 of the P value for statistical significance from the GWS for single SNP markers and for the most highly associated multi-marker haplotypes centered at the reference marker and defined by the sliding windows of specified sizes.
Table 33.2. List of significantly associated haplotypes based on the schizophrenia Disease GWS results using the Quebec Founder Population (QFP). Individual haplotypes with associated relative risks are presented in each row of the table; these values were extracted from the associated marker haplotype window with the most significant p value for each SNP in Table 33.1. The first column lists the region ID as presented in Table 1. The Haplotype column lists the specific nucleotides for the individual SNP alleles contributing to the haplotype reported. The Case and Control columns correspond to the numbers of cases and controls, respectively, containing the haplotype variant noted in the Haplotype column. The Total Case and Total Control columns list the total numbers of cases and controls for which genotype data was available for the haplotype in question. The RR column gives to the relative risk for each particular haplotype. The remainder of the columns lists the SeqIDs for the SNPs contributing to the haplotype and their relative location with respect to the central marker. The Central marker (0) column lists the SeqID for the central marker on which the haplotype is based. Flanking markers are identified by minus (−) or plus (+) signs to indicate the relative location of flanking SNPs.
Table 34.1. Genome wide association study results in the Quebec Founder Population (QFP). SNP markers found to be associated with schizophrenia from the analysis of genome wide scan (GWS) data: PTPRD-1_cr1-has_w1. Columns include: Region ID; Chromosome; Build 36 location in base pairs (bp); rs#, dbSNP data base (NCBI) reference number; Sequence ID, unique numerical identifier for this patent application; Sequence, 21 by of sequence covering 10 base pair of unique sequence flanking either side of central polymorphic SNP; −log 10 P values for GWS, −log 10 of the P value for statistical significance from the GWS for single SNP markers.
Table 34.2. List of significantly associated haplotypes based on the schizophrenia Disease GWS results using the Quebec Founder Population (QFP). Individual haplotypes with associated relative risks are presented in each row of the table; these values were extracted from the associated marker haplotype window with the most significant p value for each SNP in Table 34.1. The first column lists the region ID as presented in Table 1. The Haplotype column lists the specific nucleotides for the individual SNP alleles contributing to the haplotype reported. The Case and Control columns correspond to the numbers of cases and controls, respectively, containing the haplotype variant noted in the Haplotype column. The Total Case and Total Control columns list the total numbers of cases and controls for which genotype data was available for the haplotype in question. The RR column gives to the relative risk for each particular haplotype. The remainder of the columns lists the SeqIDs for the SNPs contributing to the haplotype and their relative location with respect to the central marker. The Central marker (0) column lists the SeqID for the central marker on which the haplotype is based. Flanking markers are identified by minus (−) or plus (+) signs to indicate the relative location of flanking SNPs.
Table 35.1. Genome wide association study results in the Quebec Founder Population (QFP). SNP markers found to be associated with schizophrenia from the analysis of genome wide scan (GWS) data: PTPRD-1_cr2-has_w1. Columns include: Region ID; Chromosome; Build 36 location in base pairs (bp); rs#, dbSNP data base (NCBI) reference number; Sequence ID, unique numerical identifier for this patent application; Sequence, 21 by of sequence covering 10 base pair of unique sequence flanking either side of central polymorphic SNP; −log 10 P values for GWS, −log 10 of the P value for statistical significance from the GWS for single SNP markers.
Table 35.2. List of significantly associated haplotypes based on the schizophrenia Disease GWS results using the Quebec Founder Population (QFP). Individual haplotypes with associated relative risks are presented in each row of the table; these values were extracted from the associated marker haplotype window with the most significant p value for each SNP in Table 35.1. The first column lists the region ID as presented in Table 1. The Haplotype column lists the specific nucleotides for the individual SNP alleles contributing to the haplotype reported. The Case and Control columns correspond to the numbers of cases and controls, respectively, containing the haplotype variant noted in the Haplotype column. The Total Case and Total Control columns list the total numbers of cases and controls for which genotype data was available for the haplotype in question. The RR column gives to the relative risk for each particular haplotype. The remainder of the columns lists the SeqIDs for the SNPs contributing to the haplotype and their relative location with respect to the central marker. The Central marker (0) column lists the SeqID for the central marker on which the haplotype is based. Flanking markers are identified by minus (−) or plus (+) signs to indicate the relative location of flanking SNPs.
Table 36. Probes used for the in situ hybridization (ISH) study (see Example section for details).
Table 37. Description of Primer sequences used for the semi-quantitative gene expression profiling by RT-PCR (see Example section for details).
Table 38. PCR product sequences.

### DEFINITIONS

Throughout the description of the present invention, several terms are used that are specific to the science of this field. For the sake of clarity and to avoid any misunderstanding, these definitions are provided to aid in the understanding of the specification and claims.

Allele: One of a pair, or series, of forms of a gene or non-genic region that occur at a given locus in a chromosome. Alleles are symbolized with the same basic symbol (e.g., B for dominant and b for recessive; B1, B2, Bn for n additive alleles at a locus). In a normal diploid cell there are two alleles of any one gene (one from each parent), which occupy the same relative position (locus) on homologous chromosomes. Within a population there may be more than two alleles of a gene. See multiple alleles. SNPs also have alleles, i.e., the two (or more) nucleotides that characterize the SNP.
Amplification of nucleic acids: refers to methods such as polymerase chain reaction (PCR), ligation amplification (or ligase chain reaction, LCR) and amplification methods based on the use of Q-beta replicase. These methods are well known in the art and are described, for example, in U.S. Pat. Nos. 4,683,195 and 4,683,202. Reagents and hardware for conducting PCR are commercially available. Primers useful for amplifying sequences from the disorder region are preferably complementary to, and preferably hybridize specifically to, sequences in the disorder region or in regions that flank a target region therein. Genes from Tables 2-4 generated by amplification may be sequenced directly. Alternatively, the amplified sequence(s) may be cloned prior to sequence analysis.
Antigenic component: is a moiety that binds to its specific antibody with sufficiently high affinity to form a detectable antigen-antibody complex.
Antibodies: refer to polyclonal and/or monoclonal antibodies and fragments thereof, and immunologic binding equivalents thereof, that can bind to proteins and fragments thereof or to nucleic acid sequences from the disorder region, particularly from the disorder gene products or a portion thereof. The term antibody is used both to refer to a homogeneous molecular entity, or a mixture such as a serum product made up of a plurality of different molecular entities. Proteins may be prepared synthetically in a protein synthesizer and coupled to a carrier molecule and injected over several months into rabbits. Rabbit sera are tested for immunoreactivity to the protein or fragment. Monoclonal antibodies may be made by injecting mice with the proteins, or fragments thereof. Monoclonal antibodies can be screened by ELISA and tested for specific immunoreactivity with protein or fragments thereof (Harlow et al. 1988, Antibodies: A Laboratory Manual, Cold Spring Harbor Laboratory, Cold Spring Harbor, N.Y.). These antibodies will be useful in developing assays as well as therapeutics.
Associated allele: refers to an allele at a polymorphic locus that is associated with a particular phenotype of interest, e.g., a predisposition to a disorder or a particular drug response.
cDNA: refers to complementary or copy DNA produced from an RNA template by the action of RNA-dependent DNA polymerase (reverse transcriptase). Thus, a cDNA clone means a duplex DNA sequence complementary to an RNA molecule of interest, included in a cloning vector or PCR amplified. This term includes genes from which the intervening sequences have been removed.
cDNA library: refers to a collection of recombinant DNA molecules containing cDNA inserts that together comprise essentially all of the expressed genes of an organism or tissue. A cDNA library can be prepared by methods known to one skilled in the art (see, e.g., Cowell and Austin, 1997, “DNA Library Protocols,” Methods in Molecular Biology). Generally, RNA is first isolated from the cells of the desired organism, and the RNA is used to prepare cDNA molecules.
Cloning: refers to the use of recombinant DNA techniques to insert a particular gene or other DNA sequence into a vector molecule. In order to successfully clone a desired gene, it is necessary to use methods for generating DNA fragments, for joining the fragments to vector molecules, for introducing the composite DNA molecule into a host cell in which it can replicate, and for selecting the clone having the target gene from amongst the recipient host cells.
Cloning vector: refers to a plasmid or phage DNA or other DNA molecule that is able to replicate in a host cell. The cloning vector is typically characterized by one or more endonuclease recognition sites at which such DNA sequences may be cleaved in a determinable fashion without loss of an essential biological function of the DNA, and which may contain a selectable marker suitable for use in the identification of cells containing the vector.
Coding sequence or a protein-coding sequence: is a polynucleotide sequence capable of being transcribed into mRNA and/or capable of being translated into a polypeptide or peptide. The boundaries of the coding sequence are typically determined by a translation start codon at the 5′-terminus and a translation stop codon at the 3′-terminus.
Complement of a nucleic acid sequence: refers to the antisense sequence that participates in Watson-Crick base-pairing with the original sequence.
Disorder region: refers to the portions of the human chromosomes displayed in Table 1 bounded by the markers from Tables 2-35.
Disorder-associated nucleic acid or polypeptide sequence: refers to a nucleic acid sequence that maps to region of Table 1 or the polypeptides encoded therein (Tables 2-4, nucleic acids, and polypeptides). For nucleic acids, this encompasses sequences that are identical or complementary to the gene sequences from Tables 2-4, as well as sequence-conservative, function-conservative, and non-conservative variants thereof. For polypeptides, this encompasses sequences that are identical to the polypeptide, as well as function-conservative and non-conservative variants thereof. Included are the alleles of naturally-occurring polymorphisms causative of SCHIZOPHRENIA disease such as, but not limited to, alleles that cause altered expression of genes of Tables 2-4 and alleles that cause altered protein levels or stability (e.g., decreased levels, increased levels, expression in an inappropriate tissue type, increased stability, and decreased stability).
Expression vector: refers to a vehicle or plasmid that is capable of expressing a gene that has been cloned into it, after transformation or integration in a host cell. The cloned gene is usually placed under the control of (i.e., operably linked to) a regulatory sequence.
Function-conservative variants: are those in which a change in one or more nucleotides in a given codon position results in a polypeptide sequence in which a given amino acid residue in the polypeptide has been replaced by a conservative amino acid substitution. Function-conservative variants also include analogs of a given polypeptide and any polypeptides that have the ability to elicit antibodies specific to a designated polypeptide.
Founder population: Also a population isolate, this is a large number of people who have mostly descended, in genetic isolation from other populations, from a much smaller number of people who lived many generations ago.
Gene: Refers to a DNA sequence that encodes through its template or messenger RNA a sequence of amino acids characteristic of a specific peptide, polypeptide, or protein. The term “gene” also refers to a DNA sequence that encodes an RNA product. The term gene as used herein with reference to genomic DNA includes intervening, non-coding regions, as well as regulatory regions, and can include 5′ and 3′ ends. A gene sequence is wild-type if such sequence is usually found in individuals unaffected by the disorder or condition of interest. However, environmental factors and other genes can also play an important role in the ultimate determination of the disorder. In the context of complex disorders involving multiple genes (oligogenic disorder), the wild type, or normal sequence can also be associated with a measurable risk or susceptibility, receiving its reference status based on its frequency in the general population.
GeneMaps: are defined as groups of gene(s) that are directly or indirectly involved in at least one phenotype of a disorder (some non-limiting example of GeneMaps comprises varius combinations of genes from Tables 2-4). As such, GeneMaps enable the development of synergistic diagnostic products, creating “theranostics”.
Genotype: Set of alleles at a specified locus or loci.
Haplotype: The allelic pattern of a group of (usually contiguous) DNA markers or other polymorphic loci along an individual chromosome or double helical DNA segment. Haplotypes identify individual chromosomes or chromosome segments. The presence of shared haplotype patterns among a group of individuals implies that the locus defined by the haplotype has been inherited, identical by descent (IBD), from a common ancestor. Detection of identical by descent haplotypes is the basis of linkage disequilibrium (LD) mapping. Haplotypes are broken down through the generations by recombination and mutation. In some instances, a specific allele or haplotype may be associated with susceptibility to a disorder or condition of interest, e.g., SCHIZOPHRENIA disease. In other instances, an allele or haplotype may be associated with a decrease in susceptibility to a disorder or condition of interest, i.e., a protective sequence.
Host: includes prokaryotes and eukaryotes. The term includes an organism or cell that is the recipient of an expression vector (e.g., autonomously replicating or integrating vector).
Hybridizable: nucleic acids are hybridizable to each other when at least one strand of the nucleic acid can anneal to another nucleic acid strand under defined stringency conditions. In some embodiments, hybridization requires that the two nucleic acids contain at least 10 substantially complementary nucleotides; depending on the stringency of hybridization, however, mismatches may be tolerated. The appropriate stringency for hybridizing nucleic acids depends on the length of the nucleic acids and the degree of complementarity, and can be determined in accordance with the methods described herein.
Identity by descent (IBD): Identity among DNA sequences for different individuals that is due to the fact that they have all been inherited from a common ancestor. LD mapping identifies IBD haplotypes as the likely location of disorder genes shared by a group of patients.
Identity: as known in the art, is a relationship between two or more polypeptide sequences or two or more polynucleotide sequences, as determined by comparing the sequences. In the art, identity also means the degree of sequence relatedness between polypeptide or polynucleotide sequences, as the case may be, as determined by the match between strings of such sequences. Identity and similarity can be readily calculated by known methods, including but not limited to those described in A. M. Lesk (ed), 1988, Computational Molecular Biology, Oxford University Press, NY; D. W. Smith (ed), 1993, Biocomputing. Informatics and Genome Projects, Academic Press, NY; A. M. Griffin and H. G. Griffin, H. G (eds), 1994, ComputerAnalysis of Sequence Data, Part 1, Humana Press, NJ; G. von Heinje, 1987, Sequence Analysis in Molecular Biology, Academic Press; and M. Gribskov and J. Devereux (eds), 1991, Sequence Analysis Primer, M Stockton Press, NY; H. Carillo and D. Lipman, 1988, SIAM J. Applied Math., 48:1073.
Immunogenic component: is a moiety that is capable of eliciting a humoral and/or cellular immune response in a host animal.
Isolated nucleic acids: are nucleic acids separated away from other components (e.g., DNA, RNA, and protein) with which they are associated (e.g., as obtained from cells, chemical synthesis systems, or phage or nucleic acid libraries). Isolated nucleic acids are at least 60% free, preferably 75% free, and most preferably 90% free from other associated components. In accordance with the present invention, isolated nucleic acids can be obtained by methods described herein, or other established methods, including isolation from natural sources (e.g., cells, tissues, or organs), chemical synthesis, recombinant methods, combinations of recombinant and chemical methods, and library screening methods.

Isolated polypeptides or peptides: are those that are separated from other components (e.g., DNA, RNA, and other polypeptides or peptides) with which they are associated (e.g., as obtained from cells, translation systems, or chemical synthesis systems). In a preferred embodiment, isolated polypeptides or peptides are at least 10% pure; more preferably, 80% or 90% pure. Isolated polypeptides and peptides include those obtained by methods described herein, or other established methods, including isolation from natural sources (e.g., cells, tissues, or organs), chemical synthesis, recombinant methods, or combinations of recombinant and chemical methods. Proteins or polypeptides referred to herein as recombinant are proteins or polypeptides produced by the expression of recombinant nucleic acids. A portion as used herein with regard to a protein or polypeptide, refers to fragments of that protein or polypeptide. The fragments can range in size from 5 amino acid residues to all but one residue of the entire protein sequence. Thus, a portion or fragment can be at least 5, 5-50, 50-100, 100-200, 200-400, 400-800, or more consecutive amino acid residues of a protein or polypeptide.

Linkage disequilibrium (LD): the situation in which the alleles for two or more loci do not occur together in individuals sampled from a population at frequencies predicted by the product of their individual allele frequencies. In other words, markers that are in LD do not follow Mendel's second law of independent random segregation. LD can be caused by any of several demographic or population artifacts as well as by the presence of genetic linkage between markers. However, when these artifacts are controlled and eliminated as sources of LD, then LD results directly from the fact that the loci involved are located close to each other on the same chromosome so that specific combinations of alleles for different markers (haplotypes) are inherited together. Markers that are in high LD can be assumed to be located near each other and a marker or haplotype that is in high LD with a genetic trait can be assumed to be located near the gene that affects that trait. The physical proximity of markers can be measured in family studies where it is called linkage or in population studies where it is called linkage disequilibrium.
LD mapping: population based gene mapping, which locates disorder genes by identifying regions of the genome where haplotypes or marker variation patterns are shared statistically more frequently among disorder patients compared to healthy controls. This method is based upon the assumption that many of the patients will have inherited an allele associated with the disorder from a common ancestor (IBD), and that this allele will be in LD with the disorder gene.
Locus: a specific position along a chromosome or DNA sequence. Depending upon context, a locus could be a gene, a marker, a chromosomal band or a specific sequence of one or more nucleotides.
Minor allele frequency (MAF): the population frequency of one of the alleles for a given polymorphism, which is equal or less than 50%. The sum of the MAF and the Major allele frequency equals one.
Markers: an identifiable DNA sequence that is variable (polymorphic) for different individuals within a population. These sequences facilitate the study of inheritance of a trait or a gene. Such markers are used in mapping the order of genes along chromosomes and in following the inheritance of particular genes; genes closely linked to the marker or in LD with the marker will generally be inherited with it. Two types of markers are commonly used in genetic analysis, microsatellites and SNPs.
Microsatellite: DNA of eukaryotic cells comprising a repetitive, short sequence of DNA that is present as tandem repeats and in highly variable copy number, flanked by sequences unique to that locus.
Mutant sequence: if it differs from one or more wild-type sequences. For example, a nucleic acid from a gene listed in Tables 2-4 containing a particular allele of a single nucleotide polymorphism may be a mutant sequence. In some cases, the individual carrying this allele has increased susceptibility toward the disorder or condition of interest. In other cases, the mutant sequence might also refer to an allele that decreases the susceptibility toward a disorder or condition of interest and thus acts in a protective manner. The term mutation may also be used to describe a specific allele of a polymorphic locus.
Non-conservative variants: are those in which a change in one or more nucleotides in a given codon position results in a polypeptide sequence in which a given amino acid residue in a polypeptide has been replaced by a non-conservative amino acid substitution. Non-conservative variants also include polypeptides comprising non-conservative amino acid substitutions.
Nucleic acid or polynucleotide: purine- and pyrimidine-containing polymers of any length, either polyribonucleotides or polydeoxyribonucleotide or mixed polyribo polydeoxyribonucleotides. This includes single- and double-stranded molecules, i.e., DNA-DNA, DNA-RNA and RNA-RNA hybrids, as well as protein nucleic acids (PNA) formed by conjugating bases to an amino acid backbone. This also includes nucleic acids containing modified bases.
Nucleotide: a nucleotide, the unit of a DNA molecule, is composed of a base, a 2′-deoxyribose and phosphate ester(s) attached at the 5′ carbon of the deoxyribose. For its incorporation in DNA, the nucleotide needs to possess three phosphate esters but it is converted into a monoester in the process.
Operably linked: means that the promoter controls the initiation of expression of the gene. A promoter is operably linked to a sequence of proximal DNA if upon introduction into a host cell the promoter determines the transcription of the proximal DNA sequence(s) into one or more species of RNA. A promoter is operably linked to a DNA sequence if the promoter is capable of initiating transcription of that DNA sequence.
Ortholog: denotes a gene or polypeptide obtained from one species that has homology to an analogous gene or polypeptide from a different species.
Paralog: denotes a gene or polypeptide obtained from a given species that has homology to a distinct gene or polypeptide from that same species.
Phenotype: any visible, detectable or otherwise measurable property of an organism such as symptoms of, or susceptibility to, a disorder.
Polymorphism: occurrence of two or more alternative genomic sequences or alleles between or among different genomes or individuals at a single locus. A polymorphic site thus refers specifically to the locus at which the variation occurs. In some cases, an individual carrying a particular allele of a polymorphism has an increased or decreased susceptibility toward a disorder or condition of interest.
Portion and fragment: are synonymous. A portion as used with regard to a nucleic acid or polynucleotide refers to fragments of that nucleic acid or polynucleotide. The fragments can range in size from 8 nucleotides to all but one nucleotide of the entire gene sequence. Preferably, the fragments are at least about 8 to about 10 nucleotides in length; at least about 12 nucleotides in length; at least about 15 to about 20 nucleotides in length; at least about 25 nucleotides in length; or at least about 35 to about 55 nucleotides in length.
Probe or primer: refers to a nucleic acid or oligonucleotide that forms a hybrid structure with a sequence in a target region of a nucleic acid due to complementarity of the probe or primer sequence to at least one portion of the target region sequence.
Protein and polypeptide: are synonymous. Peptides are defined as fragments or portions of polypeptides, preferably fragments or portions having at least one functional activity (e.g., proteolysis, adhesion, fusion, antigenic, or intracellular activity) as the complete polypeptide sequence.
Recombinant nucleic acids: nucleic acids which have been produced by recombinant DNA methodology, including those nucleic acids that are generated by procedures which rely upon a method of artificial replication, such as the polymerase chain reaction (PCR) and/or cloning into a vector using restriction enzymes. Portions of recombinant nucleic acids which code for polypeptides can be identified and isolated by, for example, the method of M. Jasin et al., U.S. Pat. No. 4,952,501.
Regulatory sequence: refers to a nucleic acid sequence that controls or regulates expression of structural genes when operably linked to those genes. These include, for example, the lac systems, the trp system, major operator and promoter regions of the phage lambda, the control region of fd coat protein and other sequences known to control the expression of genes in prokaryotic or eukaryotic cells. Regulatory sequences will vary depending on whether the vector is designed to express the operably linked gene in a prokaryotic or eukaryotic host, and may contain transcriptional elements such as enhancer elements, termination sequences, tissue-specificity elements and/or translational initiation and termination sites.
Sample: as used herein refers to a biological sample, such as, for example, tissue or fluid isolated from an individual or animal (including, without limitation, plasma, serum, cerebrospinal fluid, lymph, tears, nails, hair, saliva, milk, pus, and tissue exudates and secretions) or from in vitro cell culture-constituents, as well as samples obtained from, for example, a laboratory procedure.
Single nucleotide polymorphism (SNP): variation of a single nucleotide. This includes the replacement of one nucleotide by another and deletion or insertion of a single nucleotide. Typically, SNPs are biallelic markers although tri- and tetra-allelic markers also exist. For example, SNP ANC may comprise allele C or allele A (Tables 5-35). Thus, a nucleic acid molecule comprising SNP ANC may include a C or A at the polymorphic position. For clarity purposes, an ambiguity code is used in Tables 5-35 and the sequence listing, to represent the variations. For a combination of SNPs, the term “haplotype” is used, e.g. the genotype of the SNPs in a single DNA strand that are linked to one another. In certain embodiments, the term “haplotype” is used to describe a combination of SNP alleles, e.g., the alleles of the SNPs found together on a single DNA molecule. In specific embodiments, the SNPs in a haplotype are in linkage disequilibrium with one another.
Sequence-conservative: variants are those in which a change of one or more nucleotides in a given codon position results in no alteration in the amino acid encoded at that position (i.e., silent mutation).
Substantially homologous: a nucleic acid or fragment thereof is substantially homologous to another if, when optimally aligned (with appropriate nucleotide insertions and/or deletions) with the other nucleic acid (or its complementary strand), there is nucleotide sequence identity in at least 60% of the nucleotide bases, usually at least 70%, more usually at least 80%, preferably at least 90%, and more preferably at least 95-98% of the nucleotide bases. Alternatively, substantial homology exists when a nucleic acid or fragment thereof will hybridize, under selective hybridization conditions, to another nucleic acid (or a complementary strand thereof). Selectivity of hybridization exists when hybridization which is substantially more selective than total lack of specificity occurs. Typically, selective hybridization will occur when there is at least about 55% sequence identity over a stretch of at least about nine or more nucleotides, preferably at least about 65%, more preferably at least about 75° k, and most preferably at least about 90% (M. Kanehisa, 1984, Nucl. Acids Res. 11:203-213). The length of homology comparison, as described, may be over longer stretches, and in certain embodiments will often be over a stretch of at least 14 nucleotides, usually at least 20 nucleotides, more usually at least 24 nucleotides, typically at least 28 nucleotides, more typically at least 32 nucleotides, and preferably at least 36 or more nucleotides.
Wild-type gene from Tables 2-4: refers to the reference sequence. The wild-type gene sequences from Tables 2-4 used to identify the variants (polymorphisms, alleles, and haplotypes) described in detail herein.

Technical and scientific terms used herein have the meanings commonly understood by one of ordinary skill in the art to which the present invention pertains, unless otherwise defined. Reference is made herein to various methodologies known to those of skill in the art. Publications and other materials setting forth such known methodologies to which reference is made are incorporated herein by reference in their entireties as though set forth in full. Standard reference works setting forth the general principles of recombinant DNA technology include J. Sambrook et al., 1989, Molecular Cloning: A Laboratory Manual, 2d Ed., Cold Spring Harbor Laboratory Press, Cold Spring Harbor, N.Y.; P. B. Kaufman et al., (eds), 1995, Handbook of Molecular and Cellular Methods in Biology and Medicine, CRC Press, Boca Raton; M. J. McPherson (ed), 1991, Directed Mutagenesis: A Practical Approach, IRL Press, Oxford; J. Jones, 1992, Amino Acid and Peptide Synthesis, Oxford Science Publications, Oxford; B. M. Austen and O. M. R. Westwood, 1991, Protein Targeting and Secretion, IRL Press, Oxford; D. N Glover (ed), 1985, DNA Cloning, Volumes I and 11; M. J. Gait (ed), 1984, Oligonucleotide Synthesis; B. D. Hames and S. J. Higgins (eds), 1984, Nucleic Acid Hybridization; Quirke and Taylor (eds), 1991, PCR-A Practical Approach; Harries and Higgins (eds), 1984, Transcription and Translation; R. I. Freshney (ed), 1986, Animal Cell Culture; Immobilized Cells and Enzymes, 1986, IRL Press; Perbal, 1984, A Practical Guide to Molecular Cloning, J. H. Miller and M. P. Calos (eds), 1987, Gene Transfer Vectors for Mammalian Cells, Cold Spring Harbor Laboratory Press; M. J. Bishop (ed), 1998, Guide to Human Genome Computing, 2d Ed., Academic Press, San Diego, Calif.; L. F. Peruski and A. H. Peruski, 1997, The Internet and the New Biology. Tools for Genomic and Molecular Research, American Society for Microbiology, Washington, D.C. Standard reference works setting forth the general principles of immunology include S. Sell, 1996, Immunology, Immunopathology & Immunity, 5th Ed., Appleton & Lange, Publ., Stamford, Conn.; D. Male et al., 1996, Advanced Immunology, 3d Ed., Times Mirror Intl Publishers Ltd., Publ., London; D. P. Stites and A. L Terr, 1991, Basic and Clinical Immunology, 7th Ed., Appleton & Lange, Publ., Norwalk, Conn.; and A. K. Abbas et al., 1991, Cellular and Molecular Immunology, W. B. Saunders Co., Publ., Philadelphia, Pa. Any suitable materials and/or methods known to those of skill can be utilized in carrying out the present invention; however, preferred materials and/or methods are described. Materials, reagents, and the like to which reference is made in the following description and examples are generally obtainable from commercial sources, and specific vendors are cited herein.

### BRIEF DESCRIPTION OF THE DRAWINGS

FIG. 1. Mouse mRNA localization matrix applied to single and multiple mRNA localization assessment & comparative studies, cresyl violet staining. Slide 1 to 7: All-Stage, Whole-Body Sections throughout the embryonic (1 and 2), postnatal developmental stages (3 and 5) and adulthood (6 and 7). Slide 8: Adult Mouse Reproductive Organs: 1. Uterus, control; 2. Uterus, gestation day 5.5; 3. Uterus, gestation day 7.5; 4. Ovary; 5. Mammary gland; 6. Prostate; 7. Epididymis; 8. Testis; 9. Seminal vesicle; Slide 9: Adult Mouse Tissue Array, General: 10. Brain, sagittal sections; 11. Thyroid; 12. Pituitary gland; 13. Adrenal gland; 14. Trigeminal ganglion; 15. Ovary; 16. Uterus; 17. Kidney; 18. Testis; 19. Thymus; 20. Seminal vesicle; 21. Salivary gland; 22. Urinary Bladder; 23. Lung; 24. Prostate; 25. Liver; 26. Gallbladder; 27. Epididymis; 28. Adipose tissue; Slide 10: Adult Mouse Brain Arrays

FIG. 2. KMO expression in the embryonic (e10.5, e12.5 and e15.5) and postnatal (p1 and p10) mice. A to D) X-ray film autoradiography following hybridization with antisense riboprobe (Seq ID: 19612) after 4-day exposure, showing a pattern of Kmo mRNA distribution seen as bright labeling on dark field. E) Control (sense, Seq ID: 19611) hybridization of the section comparable to D. Abbreviations: K—kidney; Li—liver; Re—retina; Sp—spleen; (s)—sense. Magnification ×1.6.

FIG. 3. KMO expression in the adult mouse. A) Anatomical view of the adult mouse after staining with cresyl violet. B) X-ray film autoradiography after hybridization with antisense riboprobe (Seq ID: 19612) showing the presence of Kmo mRNA in the liver, spleen, lymph nodes and kidney. C) Control (sense, Seq ID: 19611) hybridization of an adjacent section comparable to B. Abbreviations: Cx—cortex, kidney; K—kidney; Li—liver; LN—lymph nodes; OMe—outer medulla, kidney; Th—thymus; (as)—antisense; (s)—sense. Magnification ×2.7

FIG. 4. KMO expression in the adult mouse tissue arrays. A) Two-day X-ray film autoradiography after hybridization with antisense riboprobe (Seq ID: 19612) showing Kmo mRNA detection in the reproductive organs (RO) seen as bright labeling on dark field. There is no evidence of mRNA labeling in these tissues. B) Kmo mRNA shown in the general tissue array (TA). Kmo expression is detectable in the spleen, kidney and liver. C) Kmo mRNA in the brain tissue arrays. Medium to high level mRNA concentration with exception of the striatum. D) Control (sense, Seq ID: 19611) hybridization of the section comparable to B. Abbreviations: BA—brain arrays; Cx—kidney cortex; K—kidney; Li—liver; Me—kidney medulla; RO—reproductive organs; TA—tissue arrays; (s)—sense. Magnification ×1.6.

FIG. 5. KMO expression in the adult mouse whole body section of the liver and lymphatic node. A) Emulsion autoradiography after hybridization with antisense riboprobe (Seq ID: 19612) showing Kmo mRNA labelling in the liver and lymph node seen as bright on darkfield illumination. B) The same fragment as in (A) seen under lightfield illumination, cresyl violet staining. C) Control (sense, Seq ID: 19611) hybridization of an adjacent section comparable to A under darkfield illumination. D) The same fragment as in (C) seen under lightfield illumination, cresyl violet staining. E) Liver at higher magnification. Large arrow indicates labelled hepatocytes. F) Control (sense, Seq ID: 19611) hybridization in the liver cells at high magnification. Abbreviations: In intestine tissue; Li—liver; LN—lymph node; (s)—sense. Magnifications: (A to D) ×54; (E and F) ×540.

FIG. 6. KMO expression in the adult mouse spleen. A) Emulsion autoradiography after hybridization with antisense riboprobe (Seq ID: 19612) showing ubiquitous Kmo mRNA labelling in the spleen seen as bright on darkfield illumination. B) The same fragment as in (A) seen under lightfield illumination, cresyl violet staining. C) Control (sense, Seq ID: 19611) hybridization of an adjacent section comparable to A under darkfield illumination. D) The same fragment as in (C) seen under lightfield illumination, cresyl violet staining. E) Spleen at higher magnification. Kmo mRNA labeling seems to follow cell density. F) Control (sense) hybridization in the spleen at high magnification. Abbreviations: RP—red pulp; WP—white pulp; (s)—sense. Magnifications: (A to D) ×54; (E and F) ×540.

FIG. 7. KMO expression in the adult mouse kidney cortex. A) Emulsion autoradiography after hybridization with antisense riboprobe (Seq ID: 19612) showing Kmo mRNA labelling in the cortex seen as bright on darkfield illumination. Note the tubules labeled but glomeruli free of labelling. B) The same fragment as in (A) seen under lightfield illumination, cresyl violet staining. C) Control (sense, Seq ID: 19611) hybridization of an adjacent section comparable to A under darkfield illumination. D) The same fragment as in (C) seen under lightfield illumination, cresyl violet staining. Abbreviations: Cx—kidney cortex; GI—glomerulus; (s)—sense. Magnifications: (A to D) ×54.

FIG. 8. KMO expression in the adult mouse kidney cortex. A) Emulsion autoradiography after hybridization with antisense riboprobe (Seq ID: 19612) showing Kmo mRNA labelling in the tubules of the kidney cortex seen as silver grain labeling under lightfield illumination, cresyl violet staining. Labelled tubules are seen in the proximity of the glomerulus, the later free of labeling. B) Deep cortex/outer medulla fragment seen under lightfield illumination, cresyl violet staining; the lobules are labelled. C) Control (sense, Seq ID: 19611) hybridization of an adjacent section comparable to A. D) Control (sense, Seq ID: 19611) hybridization of an adjacent section comparable to B. Abbreviations: Cp capillary; GI—glomerulus; Tu—renal tubule; (as)—antisense; (s)—sense. Magnifications: (A to D) ×540.

FIG. 9. CADM3 expression in the embryonic (e10.5, e12.5 and e15.5) and postnatal (p1 and p10) mice. A to D) X-ray film autoradiography following hybridization with antisense riboprobe (Seq ID: 19614) after 3-day exposure, showing a pattern of Cadm3 mRNA distribution seen as bright labeling on dark field. Labelling seems to be concentrated in the CNS brain and spinal and PNS trigeminal gangion and dorsal root ganglia. E) Control (sense, Seq ID: 19613) hybridization of the section comparable to D. Abbreviations: Br—brain; DRG—dorsal root ganglion; Re—retina; SC—spinal cord; Tg—trigeminal ganglion; (s)—sense. Magnification ×1.6.

FIG. 10. CADM3 expression in the adult mouse. A) Anatomical view of the adult mouse after staining with cresyl violet. B) X-ray film autoradiography after hybridization with antisense riboprobe (Seq ID: 19614) showing the presence of Cadm3 mRNA in the brain, spinal cord and dorsal root ganglia. C) Control (sense, Seq ID: 19613) hybridization of an adjacent section comparable to B. Abbreviations: Br—brain; Cb—cerebellum; Cx—cortex, DRG—dorsal root ganglion; H—heart; Li—liver; LI—large intestine; SI—small intestine; Tg—trigeminal ganglion; Th—thymus; (as)—antisense; (s)—sense. Magnification ×2.7

FIG. 11. CADM3 expression in the adult mouse tissue arrays. A) Two-day X-ray film autoradiography after hybridization with antisense riboprobe (Seq ID: 19614) showing Cadm3 mRNA detection in the reproductive organs (RO) seen as bright labeling on dark field. There is evidence of light mRNA labelling in the pregnant mice uteri on day 5.5 and 7.5. B) Cadm3 mRNA shown in the general tissue array (TA). Cadm3 expression is detectable in the brain and trigeminal ganglion. Weak labeling is noted in the uterus. C) Cadm3 mRNA in the brain tissue arrays (BA). Generally high-level mRNA concentration in the brain gray matter regions. D) Control (sense, Seq ID: 19613) hybridization of the section comparable to B. Abbreviations: Br—brain; Cb—cerebellum; Hip—hippocampus; OL—olfactory lobe; TG—trigeminal ganglion; Ut—uterus; (s)—sense. Magnification ×1.6.

FIG. 12. CADM3 expression in the adult mouse brain cortex and hippocampus. A) Emulsion autoradiography after hybridization with antisense riboprobe (Seq ID: 19614) showing Cadm3 mRNA labelling in the cortex and hippocampus seen as bright on darkfield illumination. B) The same fragment as in (A) seen under lightfield illumination, cresyl violet staining. C) Control (sense, Seq ID: 19613) hybridization of an adjacent section comparable to A under darkfield illumination. D) The same fragment as in (C) seen under lightfield illumination, cresyl violet staining. E) Superficial layers of the cortex (layers I and II) at higher magnification. Large arrow indicates labelled neurons, small arrows indicate the unlabelled glial cells. F) Fragment of the area 3 of the hippocampus with labeled pyramidal neurons (large arrow) and unlabelled glial cells. Abbreviations: CA1 to CA3 hippocampus cornu ammonis area 1 to 3; α-corpus calosum; Cx I and Cx II—cortical layer I and II; (s)—sense. Magnifications: (A to D) ×19; (E and F) ×440.

FIG. 13. CADM3 expression in the cerebellum. A) Emulsion autoradiography after hybridization with antisense riboprobe (Seq ID: 19614) revealing a widespread Cadm3 mRNA labelling distribution in the cerebellum seen as bright on darkfield illumination. B) The same fragment as in (A) seen under lightfield illumination, cresyl violet staining. C) Control (sense, Seq ID: 19613) hybridization of an adjacent section comparable to A under darkfield illumination. D) The same fragment as in (C) seen under lightfield illumination, cresyl violet staining. Abbreviations: Cb—cerebellum; DCN—deep cerebellar nuclei; IC—inferior colliculus; (s)—sense. Magnifications: ×24.

FIG. 14. CADM3 expression in the adult mouse trigeminal ganglion. A) Emulsion autoradiography after hybridization with antisense riboprobe (Seq ID: 19614) showing Cadm3 mRNA labelling in the trigeminal ganglion seen as bright on darkfield illumination. Note the group of labeled neurons (arrows). B) The same fragment as in (A) seen under lightfield illumination, cresyl violet staining. C) Control (sense, Seq ID: 19613) hybridization of an adjacent section comparable to A under darkfield illumination. D) Group of labeled neurons (large arrows) seen at higher magnification. Small arrows indicated unlabelled satellite glial cells. Magnifications: (A to C) ×54, (D) ×540.

FIG. 15. CADM3 expression in the postnatal mouse plexus Auerbach. A) Emulsion autoradiography after hybridization with antisense riboprobe (Seq ID: 19614) showing Cadm3 mRNA labelling in the intestinal plexus (arrow) seen as bright under darkfield illumination. B) The same fragment as in (A) seen under lightfield illumination, cresyl violet staining. C) Control (sense, Seq ID: 19613) hybridization of an adjacent section comparable to A. D) Fragment of the intestinal wall with labelled neuron (arrow) at high magnification. Abbreviations: In—intestine; SMC—smooth muscle cells; (s)—sense. Magnifications: (A to C) ×24; (D) ×540.

FIG. 16. PTPRD expression in the embryonic (e10.5, e12.5 and e15.5) and postnatal (p1 and p10) mice. A to D) X-ray film autoradiography following hybridization with antisense riboprobe (Seq ID: 19616) after 2-day exposure, showing a pattern of Ptprd mRNA distribution seen as bright labeling on dark field. Labelling seems to be mostly concentrated in the CNS brain and spinal and PNS dorsal root ganglia. Also labeled are the kidney and retina. E) Control (sense, Seq ID: 19615) hybridization of the section comparable to D. Abbreviations: BM—bone marrow; Br—brain; CNS central nervous system; DRG—dorsal root ganglion; K—kidney; Li—liver; Ov—ovary; Re—retina; SC—spinal cord; (s)—sense. Magnification ×1.6.

FIG. 17. PTPRD expression in the adult mouse. A) Anatomical view of the adult mouse after staining with cresyl violet. B) X-ray film autoradiography after hybridization with antisense riboprobe (Seq ID: 19616) showing the presence of Ptprd mRNA in the brain, spinal cord, dorsal root ganglia, liver, kidney, small and large intestine and bone marrow. C) Control (sense, Seq ID: 19615) hybridization of an adjacent section comparable to B. Abbreviations: BM—bone marrow; Br—brain; Cb—cerebellum; DRG—dorsal root ganglion; H—heart; Li—liver; LI—large intestine; SI—small intestine; (as)—antisense; (s)—sense. Magnification ×2.7

FIG. 18. PTPRD expression in the adult mouse tissue arrays. A) Two-day X-ray film autoradiography after hybridization with antisense riboprobe (Seq ID: 19616) showing Ptprd mRNA detection in the reproductive organs (RO) seen as bright labeling on dark field. There is evidence of mRNA labelling in the ovary. B) Ptprd mRNA shown in the general tissue array (TA). Ptprd expression is detectable in the brain, trigeminal ganglion, adrenal gland, pituitary, kidney, ovary and liver. Weak labeling is noted in the testis. C) Ptprd mRNA in the brain tissue arrays (BA). Heterogeneous distribution mRNA in the brain gray matter regions. D) Control (sense, Seq ID: 19615) hybridization of the section comparable to B. Abbreviations: Adr—adrenal gland; Br—brain; Cx cerebral cortex; Hip—hippocampus; K—kidney; Li—liver; Ov—ovary; Pit—pituitary gland; Rt—reticular thalamic nucleus; T—testis; TG—trigeminal ganglion; (s)—sense. Magnification ×1.6.

FIG. 19. PTPRD expression in the adult mouse brain cortex and hippocampus. A) Emulsion autoradiography after hybridization with antisense riboprobe (Seq ID: 19616) showing Ptprd mRNA labelling in the cortex and hippocampus seen as bright on darkfield illumination. Pronounced labeling can be seen in the hippocampal area CA2. B) The same fragment as in (A) seen under lightfield illumination, cresyl violet staining. C) Control (sense, Seq ID: 19615) hybridization of an adjacent section comparable to A under darkfield illumination. D) Fragment of the area 2 and 3 of the hippocampus with labelled pyramidal neurons. Abbreviations: III—3^{rd} ventricle; CA1 and CA2—hippocampus cornu ammonis area 1 and 2; cc—corpus callosum; Cx—cortex; DG—dentate gyrus; Hip—hippocampus; (s)—sense. Magnifications: (A to D) ×20; (E and F) ×460.

FIG. 20. PTPRD expression in the reticular thalamic nucleus. A) Emulsion autoradiography after hybridization with antisense riboprobe (Seq ID: 19616) revealing a Ptprd mRNA labelling in the reticular thalamic nucleus, hippocampus area CA2 and subiculum seen as bright on darkfield illumination. B) The same fragment as in (A) seen under lightfield illumination, cresyl violet staining. C) Control (sense, Seq ID: 19615) hybridization of an adjacent section comparable to A under darkfield illumination. D) Fragment of the thalamic reticular nucleus with multiple labelled neurons. Abbreviations: CA2—cornu Ammonis area 2 of the hippocampus; Cx—cortex; Hb—habenula; Hip—hippocampus; Rt—reticular thalamic nucleus; Sc—subiculum; Th—thalamus; (s)—sense. Magnifications: (A to C) ×25; (D) ×380.

FIG. 21. PTPRD expression in the olfactory lobe, cortex, cerebellum and corpos callosum. A) Emulsion autoradiography after hybridization with antisense riboprobe (Seq ID: 19616) showing Ptprd mRNA labelling in the olfactory lobe. Heavy arrow points into a mitral cells layer. B) Cerebral cortex displaying numerous population of neurons with medium-level labeling (medium arrow). C) Cerebellum with Purkinje cells layer, unlabelled (long thin arrow). D) Corpus callosum white matter with oligodendrocytes recognizable by their characteristic topography (small arrows). Magnifications: (A to C) ×25, (D) ×380.

FIG. 22. PTPRD expression in the adult mouse adrenal gland. A) Emulsion autoradiography after hybridization with antisense riboprobe (Seq ID: 19616) showing Ptprd mRNA labelling in the adrenal gland cortex seen on darkfield illumination. Arrow points into cortical region containing aldosteron synthesizing cells. B) The same fragment as in (A) seen under lightfield illumination, cresyl violet staining. C) Control (sense, Seq ID: 19615) hybridization of an adjacent section comparable to A under darkfield illumination. D) Fragment of the cortex with labeled cells in the aldosteron synthesis region (large arrows). Abbreviations: Cx—adrenal cortex; Me—medulla; (s)—sense. Magnifications: (A to C) ×54, (D) ×380.

FIG. 23. PTPRD expression in the adult mouse ovary. A) Emulsion autoradiography after hybridization with antisense riboprobe (Seq ID: 19616) showing Ptprd mRNA labelling in the ovary growing follicles (arrows). B) The same fragment as in (A) seen under lightfield illumination, cresyl violet staining. C) Control (sense, Seq ID: 19615) hybridization of an adjacent section comparable to A under darkfield illumination. D) Fragment of the ovary with follicular cells labelled. Abbreviations: F—follicle; FC—follicular cells; Ov—ovary; (s)—sense. Magnifications: (A to C) ×25; (D) ×380.

FIG. 24. PTPRD expression in the postnatal mouse intestine. A) Emulsion autoradiography after hybridization with antisense riboprobe (Seq ID: 19616) showing Ptprd mRNA labelling in the intestine seen on darkfield illumination. Arrow points labeled intestinal villi. B) The same fragment as in (A) seen under lightfield illumination, cresyl violet staining. C) Control (sense, Seq ID: 19615) hybridization of an adjacent section comparable to A under darkfield illumination. D) Fragment of the villus with labeled epithelial cells (arrow). Abbreviations: Ep—epithelium; SI—small intestine; (s)—sense. Magnifications: (A to C) ×25, (D) ×380.

FIG. 25. TMEFF2 expression in the embryonic (e10.5, e12.5 and e15.5) and postnatal (p1 and p10) mice. A to D) X-ray film autoradiography following hybridization with antisense riboprobe (Seq ID: 19618) after 4-day exposure, showing a pattern of Tmeff2 mRNA distribution seen as bright labeling on dark field. Labelling seems to be mostly concentrated in the CNS brain and spinal and PNS trigeminal gangion, stellar ganglion and dorsal root ganglia. Also labeled are the membranous structures and the plexus Auerbach in the intestinal wall. E) Control (sense, Seq ID: 19617) hybridization of the section comparable to D. Abbreviations: Au—Auerbach plexus; Br—brain; Cb—cerebellum; Cx—cerebral cortex; DRG—dorsal root ganglion; Mb—membranes; SC—spinal cord; SG—stellar ganglion; TG—trigeminal ganglion; (s)—sense. Magnification ×1.6.

FIG. 26. TMEFF2 expression in the adult mouse. A) Anatomical view of the adult mouse after staining with cresyl violet. B) X-ray film autoradiography after hybridization with antisense riboprobe (Seq ID: 19618) showing the presence of Tmeff2 mRNA in the brain, spinal cord and dorsal root ganglia. C) Control (sense, Seq ID: 19617) hybridization of an adjacent section comparable to B. Note non-specific labeling in the blood vessels (asterisk). Abbreviations: Br—brain; Cb—cerebellum; Cx—cortex, DRG—dorsal root ganglion; H—heart; Li—liver; Tg—trigeminal ganglion; Th—thymus; (as)—antisense; (s)—sense. Magnification ×2.7

FIG. 27. TMEFF2 expression in the adult mouse tissue arrays. A) Two-day X-ray film autoradiography after hybridization with antisense riboprobe (Seq ID: 19618) showing Tmeff2 mRNA detection in the reproductive organs (RO) seen as bright labeling on dark field. There is evidence of light mRNA labelling in the ovary. B) Tmeff2 mRNA shown in the general tissue array (TA). Tmeff2 expression is detectable in the brain, trigeminal ganglion and adrenal gland. Weak labeling is noted in the uterus. C) Tmeff2 mRNA in the brain tissue arrays (BA). Generally high-level mRNA concentration in the brain gray matter regions. D) Control (sense, Seq ID: 19617) hybridization of the section comparable to B. Abbreviations: Br—brain; Cb—cerebellum; Hb—habenula; Hip—hippocampus; Ov—ovary; TG—trigeminal ganglion; Ut—uterus; (s)—sense. Magnification ×1.6.

FIG. 28. TMEFF2 expression in the adult mouse brain cortex and hippocampus. A) Emulsion autoradiography after hybridization with antisense riboprobe (Seq ID: 19618) showing Tmeff2 mRNA labelling in the cortex and hippocampus seen as bright on darkfield illumination. B) The same fragment as in (A) seen under lightfield illumination, cresyl violet staining. C) Control (sense, Seq ID: 19617) hybridization of an adjacent section comparable to A under darkfield illumination. D) The same fragment as in (C) seen under lightfield illumination, cresyl violet staining. E) Layer IV of the cortex at higher magnification. Large arrow indicates labelled neuron, small arrows point into unlabelled neurons, asterisks indicate glial cells free of labelling. F) Fragment of the area 3 of the hippocampus with labelled pyramidal neurons (large arrow). Some unlabelled glial cells are seen (asterisk). Abbreviations: CA1 to CA3 hippocampus cornu ammonis area 1 to 3; Cx—cortex; DG dentate gyrus; Hip—hippocampus; (s)—sense. Magnifications: (A to D) ×20; (E and F) ×460.

FIG. 29. TMEFF2 expression in the cerebellum. A) Emulsion autoradiography after hybridization with antisense riboprobe (Seq ID: 19618) revealing a widespread Tmeff2 mRNA labelling distribution in the cerebellum seen as bright on darkfield illumination. Arrow indicates Purkinje cells. B) The same fragment as in (A) seen under lightfield illumination, cresyl violet staining. C) Control (sense, Seq ID: 19617) hybridization of an adjacent section comparable to A under darkfield illumination. D) Fragment of cerebellar folia showing Purkinje cells labeled (arrows). Abbreviations: Cb—cerebellum; DCN—deep cerebellar nuclei; IC—inferior colliculus; (s)—sense. Magnifications: (A to C) ×23; (D) ×540.

FIG. 30. TMEFF2 expression in the adult mouse trigeminal ganglion. A) Emulsion autoradiography after hybridization with antisense riboprobe (Seq ID: 19618) showing Tmeff2 mRNA labelling in the trigeminal ganglion seen as bright on darkfield illumination. Arrow points into a group of labeled neurons. B) The same fragment as in (A) seen under lightfield illumination, cresyl violet staining. C) Control (sense, Seq ID: 19617) hybridization of an adjacent section comparable to A under darkfield illumination. D) Group of labeled neurons (large arrows) seen at higher magnification mixed with unlabelled neurons (small arrows). Asterisks indicate unlabelled satellite glial cells. Magnifications: (A to C) ×54, (D) ×540.

FIG. 31. TMEFF2 expression in the adult mouse adrenal gland. A) Emulsion autoradiography after hybridization with antisense riboprobe (Seq ID: 19618) showing Tmeff2 mRNA labelling in the adrenal gland medulla seen as bright on darkfield illumination. Arrow points into medulla containing adrenal-peptide synthesizing cells, cortical region containing corticoid aldosteron synthesizing cells unlabelled. B) The same fragment as in (A) seen under lightfield illumination, cresyl violet staining. C) Control (sense, Seq ID: 19617) hybridization of an adjacent section comparable to A under darkfield illumination. D) Fragment of the medulla with labeled cells (large arrows) and cortical region free of labeling. Abbreviations: Adr GI—adrenal gland; Cx—adrenal cortex; Me—medulla; (s)—sense. Magnifications: (A to C) ×54, (D) ×540.

FIG. 32. TMEFF2 expression in the postnatal mouse plexus Auerbach. A) Emulsion autoradiography after hybridization with antisense riboprobe (Seq ID: 19618) showing Tmeff2 mRNA labelling in the myenteric plexus (arrow) seen as bright under darkfield illumination. The labelling reveals a collection of ganglia (arrows) forming Auerbach's plexus, which is a main nerve supply to the gastrointestinal tract. B) The same fragment as in (A) seen under lightfield illumination, cresyl violet staining. C) Control (sense, Seq ID: 19617) hybridization of an adjacent section comparable to A under darkfield illumination. D) Control (sense, Seq ID: 19617) hybridization of an adjacent section comparable to B. E) Fragment of the intestinal wall with labelled ganglion neuron (arrow) at high magnification. F) Control (sense) hybridization of an adjacent section comparable to E under lightfield illumination. Abbreviations: In intestine; SMC smooth muscle cells; (s)—sense. Magnifications: (A to C) ×22; (D) ×500.

FIG. 33. Schizophrenia Gene Map, including analysis for Full cohort, Conditionals, Subphenotypes, and Gender Specific.

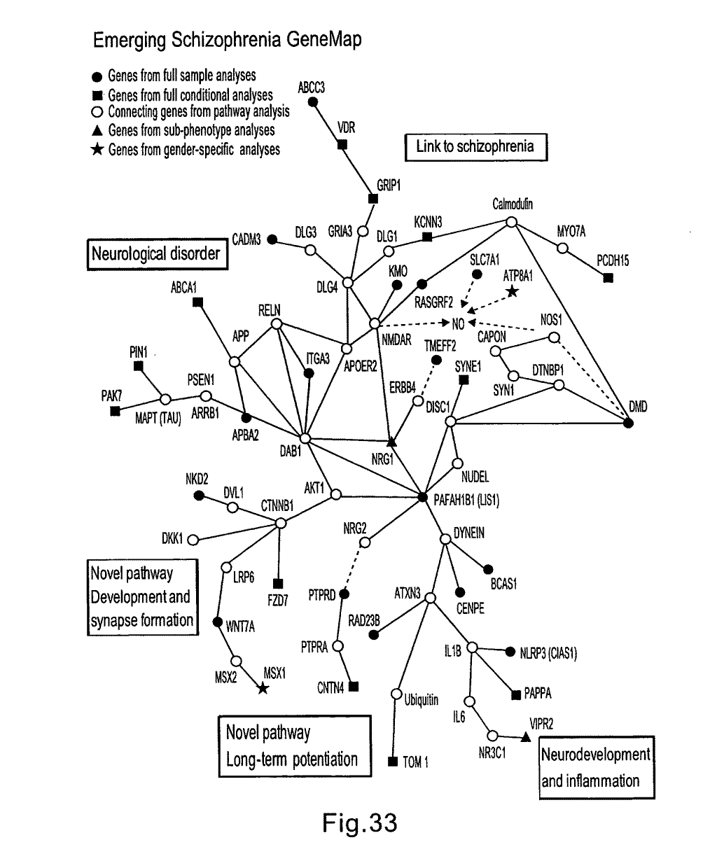

### DETAILED DESCRIPTION OF THE INVENTION

### Genome Wide Association Study to Construct a GeneMap for Schizophrenia

The present invention is based on the discovery of genes associated with SCHIZOPHRENIA disease. In the preferred embodiment, disease-associated loci (candidate regions; Table 1) are identified by the statistically significant differences in allele or haplotype frequencies between the cases and the controls.

The invention also provides a method for the discovery of genes associated with SCHIZOPHRENIA disease and the construction of a GeneMap for SCHIZOPHRENIA disease in a human population, comprising the following steps (see also Example section herein):

### Step 1: Recruit Patients (Cases) and Controls

In the preferred embodiment, 500 patients diagnosed for SCHIZOPHRENIA disease along with 500 independent controls samples are recruited from the Quebec Founder Population (QFP).

In another embodiment, more or less than 500 patients and controls are recruited.

In another embodiment, 500 patients diagnosed for SCHIZOPHRENIA disease along with two family members are recruited from the Quebec Founder Population (QFP). The preferred trios recruited are parent-parent-child (PPC) trios. Trios can also be recruited as parent-child-child (PCC) trios. In another preferred embodiment, more or less than 500 trios are recruited

In yet another embodiment, the present invention is performed as a whole or partially with DNA samples from individuals of another founder population than the Quebec population or from the general population.

### Step 2: DNA Extraction and Quantitation

Any sample comprising cells or nucleic acids from patients or controls may be used. Preferred samples are those easily obtained from the patient or control. Such samples include, but are not limited to blood, peripheral lymphocytes, buccal swabs, epithelial cell swabs, nails, hair, bronchoalveolar lavage fluid, sputum, or other body fluid or tissue obtained from an individual.

In one embodiment, DNA is extracted from such samples in the quantity and quality necessary to perform the invention using conventional DNA extraction and quantitation techniques. The present invention is not linked to any DNA extraction or quantitation platform in particular.

### Step 3: Genotype the Recruited Individuals

In one embodiment, assay-specific and/or locus-specific and/or allele-specific oligonucleotides for every SNP marker of the present invention (Tables 5-35) are organized onto one or more arrays. The genotype at each SNP locus is revealed by hybridizing short PCR fragments comprising each SNP locus onto these arrays. The arrays permit a high-throughput genome wide association study using DNA samples from individuals of the Quebec founder population. Such assay-specific and/or locus-specific and/or allele-specific oligonucleotides necessary for scoring each SNP of the present invention are preferably organized onto a solid support. Such supports can be arrayed on wafers, glass slides, beads or any other type of solid support.

In another embodiment, the assay-specific and/or locus-specific and/or allele-specific oligonucleotides are not organized onto a solid support but are still used as a whole, in panels or one by one. The present invention is therefore not linked to any genotyping platform in particular.

In another embodiment, one or more portions of the SNP maps (publicly available maps and our own proprietary QLDM map) are used to screen the whole genome, a subset of chromosomes, a chromosome, a subset of genomic regions or a single genomic region.

In the preferred embodiment, the individuals composing the cases and controls or the trios are preferably individually genotyped with at least 100,000 markers, generating at least a few million genotypes; more preferably, at least a hundred million. In another embodiment, individuals are pooled in cases and control pools for genotyping and genetic analysis.

Step 4: Exclude the Markers that Did not Pass the Quality Control of the Assay.

Preferably, the quality controls comprises, but are not limited to, the following criteria: eliminate SNPs that had a high rate of Mendelian errors (cut-off at 1% Mendelian error rate), that deviate from the Hardy-Weinberg equilibrium, that are non-polymorphic in the Quebec founder population or have too many missing data (cut-off at 1% missing values or higher), or simply because they are non-polymorphic in the Quebec founder population (cut-off at 1%≦10% minor allele frequency (MAF)).

### Step 5: Perform the Genetic Analysis on the Results Obtained Using Haplotype Information as Well as Single-Marker Association.

In the preferred embodiment, genetic analysis is performed on all the genotypes from Step 3.

In another embodiment, genetic analysis is performed on a subset of markers from Step 3 or from markers that passed the quality controls from Step 4.

In one embodiment, the genetic analysis consists of, but is not limited to features corresponding to Phase information and haplotype structures. Phase information and haplotype structures are preferably deduced from trio genotypes using Phasefinder. Since chromosomal assignment (phase) cannot be estimated when all trio members are heterozygous, an Expectation-Maximization (EM) algorithm may be used to resolve chromosomal assignment ambiguities after Phasefinder.

In yet another embodiment, the PL-EM algorithm (Partition-Ligation EM; Niu et al., Am. J. Hum. Genet. 70:157 (2002)) can be used to estimate haplotypes from the “genotype” data as a measured estimate of the reference allele frequency of a SNP in 15-marker windows that advance in increments of one marker across the data set. The results from such algorithms are converted into 15-marker haplotype files. Subsequently, the individual 15-marker block files are assembled into one continuous block of haplotypes for the entire chromosome. These extended haplotypes can then be used for further analysis. Such haplotype assembly algorithms take the consensus estimate of the allele call at each marker over all separate estimations (most markers are estimated 15 different times as the 15 marker blocks pass over their position).

In another embodiment, the haplotype frequencies among patients are compared to those among the controls using LDSTATS, a program that assesses the association of haplotypes with the disease. Such program defines haplotypes using multi-marker windows that advance across the marker map in one-marker increments. Such windows can be 1, 3, 5, 7 or 9 markers wide, and all these window sizes are tested concurrently. Larger multi-marker haplotype windows can also be used. At each position the frequency of haplotypes in cases is compared to the frequency of haplotypes in controls. Such allele frequency differences for single marker windows can be tested using Pearson's Chi-square with any degree of freedom. Multi-allelic haplotype association can be tested using Smith's normalization of the square root of Pearson's Chi-square. Such significance of association can be reported in two ways:

The significance of association within any one haplotype window is plotted against the marker that is central to that window.

P-values of association for each specific marker are calculated as a pooled P-value across all haplotype windows in which they occur. The pooled P-value is calculated using an expected value and variance calculated using a permutation test that considers covariance between individual windows. Such pooled P-values can yield narrower regions of gene location than the window data (see Example 3 herein for details on various analysis methods, such as LDSTATS v2.0 and v4.0).

In another embodiment, conditional haplotype and subtype analyses can be performed on subsets of the original set of cases and controls using the program LDSTATS. For conditional analyses, the selection of a subset of cases and their matched controls can be based on the carrier status of cases at a gene or locus of interest (see conditional analysis section in Example 3 herein). Various conditional haplotypes can be derived, such as protective haplotypes and risk haplotypes.

### Step 6: SNP and DNA Polymorphism Discovery

In the preferred embodiment, all the candidate genes and regions identified in step 5 are sequenced for polymorphism identification.

In another embodiment, the entire region, including all introns, is sequenced to identify all polymorphisms.

In yet another embodiment, the candidate genes are prioritized for sequencing, and only functional gene elements (promoters, conserved noncoding sequences, exons and splice sites) are sequenced.

In yet another embodiment, previously identified polymorphisms in the candidate regions can also be used. For example, SNPs from dbSNP, or others can also be used rather than resequencing the candidate regions to identify polymorphisms. The discovery of SNPs and DNA polymorphisms generally comprises a step consisting of determining the major haplotypes in the region to be sequenced. The preferred samples are selected according to which haplotypes contribute to the association signal observed in the region to be sequenced. The purpose is to select a set of samples that covers all the major haplotypes in the given region. Each major haplotype is preferably analyzed in at least a few individuals.

Any analytical procedure may be used to detect the presence or absence of variant nucleotides at one or more polymorphic positions of the invention. In general, the detection of allelic variation requires a mutation discrimination technique, optionally an amplification reaction and optionally a signal generation system. Any means of mutation detection or discrimination may be used. For instance, DNA sequencing, scanning methods, hybridization, extension based methods, incorporation based methods, restriction enzyme-based methods and ligation-based methods may be used in the methods of the invention.

Sequencing methods include, but are not limited to, direct sequencing, and sequencing by hybridization. Scanning methods include, but are not limited to, protein truncation test (PTT), single-strand conformation polymorphism analysis (SSCP), denaturing gradient gel electrophoresis (DGGE), temperature gradient gel electrophoresis (TGGE), cleavage, heteroduplex analysis, chemical mismatch cleavage (CMC), and enzymatic mismatch cleavage. Hybridization-based methods of detection include, but are not limited to, solid phase hybridization such as dot blots, multiple allele specific diagnostic assay (MASDA), reverse dot blots, and oligonucleotide arrays (DNA Chips). Solution phase hybridization amplification methods may also be used, such as Taqman. Extension based methods include, but are not limited to, amplification refraction mutation systems (ARMS), amplification refractory mutation systems (ALEX), and competitive oligonucleotide priming systems (COPS). Incorporation based methods include, but are not limited to, mini-sequencing and arrayed primer extension (APEX). Restriction enzyme-based detection systems include, but are not limited to, restriction site generating PCR. Lastly, ligation based detection methods include, but are not limited to, oligonucleotide ligation assays (OLA). Signal generation or detection systems that may be used in the methods of the invention include, but are not limited to, fluorescence methods such as fluorescence resonance energy transfer (FRET), fluorescence quenching, fluorescence polarization as well as other chemiluminescence, electrochemiluminescence, Raman, radioactivity, colometric methods, hybridization protection assays and mass spectrometry methods. Further amplification methods include, but are not limited to self sustained replication (SSR), nucleic acid sequence based amplification (NASBA), ligase chain reaction (LCR), strand displacement amplification (SDA) and branched DNA (B-DNA).

Sequencing can also be performed using a proprietary sequencing technology (Cantaloupe; PCT/EP2005/002870).

### Step 7: Ultrafine Mapping

This step further maps the candidate regions and genes confirmed in the previous step to identify and validate the responsible polymorphisms associated with SCHIZOPHRENIA disease in the human population.

In a preferred embodiment, the discovered SNPs and polymorphisms of step 6 are ultrafine mapped at a higher density of markers than the GWS described herein using the same technology described in step 3.

### Step 8: GeneMap Construction

The confirmed variations in DNA (including both genic and non-genic regions) are used to build a GeneMap for SCHIZOPHRENIA disease. The gene content of this GeneMap is described in more detail below. Such GeneMap can be used for other methods of the invention comprising the diagnostic methods described herein, the susceptibility to SCHIZOPHRENIA disease, the response to a particular drug, the efficacy of a particular drug, the screening methods described herein and the treatment methods described herein.

As is evident to one of ordinary skill in the art, all of the above steps or the steps do not need to be performed, or performed in a given order to practice or use the SNPs, genomic regions, genes, proteins, etc. in the methods of the invention.

Genes from the GeneMap

In one embodiment the GeneMap consists of genes and targets, in a variety of combinations, identified from the candidate regions listed in Table 1. In another embodiment, all genes from Tables 2-4 are present in the GeneMap. In another preferred embodiment, the GeneMap consists of a selection of genes from Tables 2-4. For clarity purposes, the GeneMap from the Example section herein is a not limiting example of a GeneMap. Other GeneMaps with various combinations of genes from the invention, and genes interacting with genes from the invention, can be established from the data herein.

The genes of the invention (Tables 2-4) are arranged by candidate regions and by their chromosomal location. Such order is for the purpose of clarity and does not reflect any other criteria of selection in the association of the genes with SCHIZOPHRENIA disease.

In one embodiment, genes identified in the WGAS and subsequent studies are evaluated using the Ingenuity Pathway Analysis application (IPA, Ingenuity systems) in order to identify direct biological interactions between these genes, and also to identify molecular regulators acting on those genes (indirect interactions) that could be also involved in SCHIZOPHRENIA. The purpose of this effort is to decipher the molecules involved in contributing to SCHIZOPHRENIA. These gene interaction networks are very valuable tools in the sense that they facilitate extension of the map of gene products that could represent potential drug targets for SCHIZOPHRENIA.

In another embodiment, other means (such as functional biochemical assays and genetic assays) are used to identify the biological interactions between genes to create a GeneMap (see Example section herein for description of the various GeneMaps).

### Nucleic Acid Sequences

The nucleic acid sequences of the present invention may be derived from a variety of sources including DNA, cDNA, synthetic DNA, synthetic RNA, derivatives, mimetics or combinations thereof. Such sequences may comprise genomic DNA, which may or may not include naturally occurring introns, genic regions, nongenic regions, and regulatory regions. Moreover, such genomic DNA may be obtained in association with promoter regions or poly (A) sequences. The sequences, genomic DNA, or cDNA may be obtained in any of several ways. Genomic DNA can be extracted and purified from suitable cells by means well known in the art. Alternatively, mRNA can be isolated from a cell and used to produce cDNA by reverse transcription or other means. The nucleic acids described herein are used in certain embodiments of the methods of the present invention for production of RNA, proteins or polypeptides, through incorporation into cells, tissues, or organisms. In one embodiment, DNA containing all or part of the coding sequence for the genes described in Tables 2-4, or the SNP markers described in Tables 5-35, is incorporated into a vector for expression of the encoded polypeptide in suitable host cells. The invention also comprises the use of the nucleotide sequence of the nucleic acids of this invention to identify DNA probes for the genes described in Tables 2-4 or the SNP markers described in Tables 5-35, PCR primers to amplify the genes described in Tables 2-4 or the SNP markers described in Tables 5-35, nucleotide polymorphisms in the genes described in Tables 2-4, and regulatory elements of the genes described in Tables 2-4. The nucleic acids of the present invention find use as primers and templates for the recombinant production of SCHIZOPHRENIA disease-associated peptides or polypeptides, for chromosome and gene mapping, to provide antisense sequences, for tissue distribution studies, to locate and obtain full length genes, to identify and obtain homologous sequences (wild-type and mutants), and in diagnostic applications.

### Antisense Oligonucleotides

In a particular embodiment of the invention, an antisense nucleic acid or oligonucleotide is wholly or partially complementary to, and can hybridize with, a target nucleic acid (either DNA or RNA) having the sequence of SEQ ID NO:1, NO:3 or any SEQ ID from any Tables of the invention. For example, an antisense nucleic acid or oligonucleotide comprising 16 nucleotides can be sufficient to inhibit expression of at least one gene from Tables 2-4. Alternatively, an antisense nucleic acid or oligonucleotide can be complementary to 5′ or 3′ untranslated regions, or can overlap the translation initiation codon (5′ untranslated and translated regions) of at least one gene from Tables 2-4, or its functional equivalent. In another embodiment, the antisense nucleic acid is wholly or partially complementary to, and can hybridize with, a target nucleic acid that encodes a polypeptide from a gene described in Tables 2-4.

In addition, oligonucleotides can be constructed which will bind to duplex nucleic acid (i.e., DNA:DNA or DNA:RNA), to form a stable triple helix containing or triplex nucleic acid. Such triplex oligonucleotides can inhibit transcription and/or expression of a gene from Tables 2-4, or its functional equivalent (M. D. Frank-Kamenetskii et al., 1995). Triplex oligonucleotides are constructed using the basepairing rules of triple helix formation and the nucleotide sequence of the genes described in Tables 2-4.

The present invention encompasses methods of using oligonucleotides in antisense inhibition of the function of the genes from Tables 2-4. In the context of this invention, the term “oligonucleotide” refers to naturally-occurring species or synthetic species formed from naturally-occurring subunits or their close homologs. The term may also refer to moieties that function similarly to oligonucleotides, but have non-naturally-occurring portions. Thus, oligonucleotides may have altered sugar moieties or inter-sugar linkages. Exemplary among these are phosphorothioate and other sulfur containing species which are known in the art. In preferred embodiments, at least one of the phosphodiester bonds of the oligonucleotide has been substituted with a structure that functions to enhance the ability of the compositions to penetrate into the region of cells where the RNA whose activity is to be modulated is located. It is preferred that such substitutions comprise phosphorothioate bonds, methyl phosphonate bonds, or short chain alkyl or cycloalkyl structures. In accordance with other preferred embodiments, the phosphodiester bonds are substituted with structures which are, at once, substantially non-ionic and non-chiral, or with structures which are chiral and enantiomerically specific. Persons of ordinary skill in the art will be able to select other linkages for use in the practice of the invention. Oligonucleotides may also include species that include at least some modified base forms. Thus, purines and pyrimidines other than those normally found in nature may be so employed. Similarly, modifications on the furanosyl portions of the nucleotide subunits may also be effected, as long as the essential tenets of this invention are adhered to. Examples of such modifications are 2′-O-alkyl- and 2′-halogen-substituted nucleotides. Some non-limiting examples of modifications at the 2′ position of sugar moieties which are useful in the present invention include OH, SH, SCH3, F, OCH3, OCN, O(CH2), NH2 and O(CH2)n CH3, where n is from 1 to about 10. Such oligonucleotides are functionally interchangeable with natural oligonucleotides or synthesized oligonucleotides, which have one or more differences from the natural structure. All such analogs are comprehended by this invention so long as they function effectively to hybridize with at least one gene from Tables 2-4 DNA or RNA to inhibit the function thereof.

The oligonucleotides in accordance with this invention preferably comprise from about 3 to about 50 subunits. It is more preferred that such oligonucleotides and analogs comprise from about 8 to about 25 subunits and still more preferred to have from about 12 to about 20 subunits. As defined herein, a “subunit” is a base and sugar combination suitably bound to adjacent subunits through phosphodiester or other bonds.

Antisense nucleic acids or oligonulcleotides can be produced by standard techniques (see, e.g., Shewmaker et al., U.S. Pat. No. 6,107,065). The oligonucleotides used in accordance with this invention may be conveniently and routinely made through the well-known technique of solid phase synthesis. Any other means for such synthesis may also be employed; however, the actual synthesis of the oligonucleotides is well within the abilities of the practitioner. It is also well known to prepare other oligonucleotides such as phosphorothioates and alkylated derivatives.

The oligonucleotides of this invention are designed to be hybridizable with RNA (e.g., mRNA) or DNA from genes described in Tables 2-4. For example, an oligonucleotide (e.g., DNA oligonucleotide) that hybridizes to mRNA from a gene described in Tables 2-4 can be used to target the mRNA for RnaseH digestion. Alternatively an oligonucleotide that can hybridize to the translation initiation site of the mRNA of a gene described in Tables 2-4 can be used to prevent translation of the mRNA. In another approach, oligonucleotides that bind to the double-stranded DNA of a gene from Tables 2-4 can be administered. Such oligonucleotides can form a triplex construct and inhibit the transcription of the DNA encoding polypeptides of the genes described in Tables 2-4. Triple helix pairing prevents the double helix from opening sufficiently to allow the binding of polymerases, transcription factors, or regulatory molecules. Recent therapeutic advances using triplex DNA have been described (see, e.g., J. E. Gee et al., 1994, Molecular and Immunologic Approaches, Futura Publishing Co., Mt. Kisco, N.Y.).

As non-limiting examples, antisense oligonucleotides may be targeted to hybridize to the following regions: mRNA cap region; translation initiation site; translational termination site; transcription initiation site; transcription termination site; polyadenylation signal; 3′ untranslated region; 5′ untranslated region; 5′ coding region; mid coding region; 3′ coding region; DNA replication initiation and elondation sites. Preferably, the complementary oligonucleotide is designed to hybridize to the most unique 5′ sequence of a gene described in Tables 2-4, including any of about 15-35 nucleotides spanning the 5′ coding sequence. In accordance with the present invention, the antisense oligonucleotide can be synthesized, formulated as a pharmaceutical composition, and administered to a subject. The synthesis and utilization of antisense and triplex oligonucleotides have been previously described (e.g., Simon et al., 1999; Barre et al., 2000; Elez et al., 2000; Sauter et al., 2000).

Alternatively, expression vectors derived from retroviruses, adenovirus, herpes or vaccinia viruses or from various bacterial plasmids may be used for delivery of nucleotide sequences to the targeted organ, tissue or cell population. Methods which are well known to those skilled in the art can be used to construct recombinant vectors which will express nucleic acid sequence that is complementary to the nucleic acid sequence encoding a polypeptide from the genes described in Tables 2-4. These techniques are described both in Sambrook et al., 1989 and in Ausubel et al., 1992. For example, expression of at least one gene from Tables 2-4 can be inhibited by transforming a cell or tissue with an expression vector that expresses high levels of untranslatable sense or antisense sequences. Even in the absence of integration into the DNA, such vectors may continue to transcribe RNA molecules until they are disabled by endogenous nucleases. Transient expression may last for a month or more with a nonreplicating vector, and even longer if appropriate replication elements are included in the vector system. Various assays may be used to test the ability of gene-specific antisense oligonucleotides to inhibit the expression of at least one gene from Tables 2-4. For example, mRNA levels of the genes described in Tables 2-4 can be assessed by Northern blot analysis (Sambrook et al., 1989; Ausubel et al., 1992; J. C. Alwine et al. 1977; I. M. Bird, 1998), quantitative or semi-quantitative RT-PCR analysis (see, e.g., W. M. Freeman at al., 1999; Ren et al., 1998; J. M. Cale et al., 1998), or in situ hybridization (reviewed by A. K. Raap, 1998). Alternatively, antisense oligonucleotides may be assessed by measuring levels of the polypeptide from the genes described in Tables 2-4, e.g., by western blot analysis, indirect immunofluorescence and immunoprecipitation techniques (see, e.g., J. M. Walker, 1998, Protein Protocols on cD-ROM, Humana Press, Totowa, N.J.). Any other means for such detection may also be employed, and is well within the abilities of the practitioner.

### Mapping Technologies

The present invention includes various methods which employ mapping technologies to map SNPs and polymorphisms. For purpose of clarity, this section comprises, but is not limited to, the description of mapping technologies that can be utilized to achieve the embodiments described herein. Mapping technologies may be based on amplification methods, restriction enzyme cleavage methods, hybridization methods, sequencing methods, and cleavage methods using agents.

Amplification methods include: self sustained sequence replication (Guatelli et al., 1990), transcriptional amplification system (Kwoh et al., 1989), Q-Beta Replicase (Lizardi et al., 1988), isothermal amplification (e.g. Dean et al., 2002; and Hafner et al., 2001), or any other nucleic acid amplification method, followed by the detection of the amplified molecules using techniques well known to those of ordinary skill in the art. These detection schemes are especially useful for the detection of nucleic acid molecules if such molecules are present in very low number.

Restriction enzyme cleavage methods include: isolating sample and control DNA, amplification (optional), digestion with one or more restriction endonucleases, determination of fragment length sizes by gel electrophoresis and comparing samples and controls. Differences in fragment length sizes between sample and control DNA indicates mutations in the sample DNA. Moreover, sequence specific ribozymes (see, e.g., U.S. Pat. No. 5,498,531 or DNAzyme e.g. U.S. Pat. No. 5,807,718) can be used to score for the presence of specific mutations by development or loss of a ribozyme or DNAzyme cleavage site.

Hybridization methods include any measurement of the hybridization or gene expression levels, of sample nucleic acids to probes corresponding to about 2, 3, 4, 5, 6, 7, 8, 9, 10, 15, 20, 25, 30, 50, 75, 100, 200, 500, 1000 or more genes, or ranges of these numbers, such as about 5-20, about 10-20, about 20-50, about 50-100, or about 100-200 genes of Tables 2-4.

SNPs and SNP maps of the invention can be identified or generated by hybridizing sample nucleic acids, e.g., DNA or RNA, to high density arrays or bead arrays containing oligonucleotide probes corresponding to the polymorphisms of Tables 5-35 (see the Affymetrix arrays and Illumine bead sets at www.affymetrix.com and www.illumina.com and see Cronin at al., 1996; or Kozel et al., 1996).

Methods of forming high density arrays of oligonucleotides with a minimal number of synthetic steps are known. The oligonucleotide analogue array can be synthesized on a single or on multiple solid substrates by a variety of methods, including, but not limited to, light-directed chemical coupling, and mechanically directed coupling (see Pirrung, U.S. Pat. No. 5,143,854).

In brief, the light-directed combinatorial synthesis of oligonucleotide arrays on a glass surface precedes using automated phosphoramidite chemistry and chip masking techniques. In one specific implementation, a glass surface is derivatized with a silane reagent containing a functional group, e.g., a hydroxyl or amine group blocked by a photolabile protecting group. Photolysis through a photolithogaphic mask is used selectively to expose functional groups which are then ready to react with incoming 5′ photoprotected nucleoside phosphoramidites. The phosphoramidites react only with those sites which are illuminated (and thus exposed by removal of the photolabile blocking group). Thus, the phosphoramidites only add to those areas selectively exposed from the preceding step. These steps are repeated until the desired array of sequences have been synthesized on the solid surface. Combinatorial synthesis of different oligonucleotide analogues at different locations on the array is determined by the pattern of illumination during synthesis and the order of addition of coupling reagents.

In addition to the foregoing, additional methods which can be used to generate an array of oligonucleotides on a single substrate are described in PCT Publication Nos. WO 93/09668 and WO 01/23614. High density nucleic acid arrays can also be fabricated by depositing pre-made or natural nucleic acids in predetermined positions. Synthesized or natural nucleic acids are deposited on specific locations of a substrate by light directed targeting and oligonucleotide directed targeting. Another embodiment uses a dispenser that moves from region to region to deposit nucleic acids in specific spots.

Nucleic acid hybridization simply involves contacting a probe and target nucleic acid under conditions where the probe and its complementary target can form stable hybrid duplexes through complementary base pairing. See WO 99/32660. The nucleic acids that do not form hybrid duplexes are then washed away leaving the hybridized nucleic acids to be detected, typically through detection of an attached detectable label. It is generally recognized that nucleic acids are denatured by increasing the temperature or decreasing the salt concentration of the buffer containing the nucleic acids. Under low stringency conditions (e.g., low temperature and/or high salt) hybrid duplexes (e.g., DNA:DNA, RNA:RNA, or RNA:DNA) will form even where the annealed sequences are not perfectly complementary. Thus, specificity of hybridization is reduced at lower stringency. Conversely, at higher stringency (e.g., higher temperature or lower salt) successful hybridization tolerates fewer mismatches. One of skill in the art will appreciate that hybridization conditions may be selected to provide any degree of stringency.

In a preferred embodiment, hybridization is performed at low stringency to ensure hybridization and then subsequent washes are performed at higher stringency to eliminate mismatched hybrid duplexes. Successive washes may be performed at increasingly higher stringency until a desired level of hybridization specificity is obtained. Stringency can also be increased by addition of agents such as formamide. Hybridization specificity may be evaluated by comparison of hybridization to the test probes with hybridization to the various controls that can be present (e.g., expression level control, normalization control, mismatch controls, etc.).

In general, there is a tradeoff between hybridization specificity (stringency) and signal intensity. Thus, in a preferred embodiment, the wash is performed at the highest stringency that produces consistent results and that provides a signal intensity greater than approximately 10% of the background intensity. Thus, in a preferred embodiment, the hybridized array may be washed at successively higher stringency solutions and read between each wash. Analysis of the data sets thus produced will reveal a wash stringency above which the hybridization pattern is not appreciably altered and which provides adequate signal for the particular oligonucleotide probes of interest.

Probes based on the sequences of the genes described above may be prepared by any commonly available method. Oligonucleotide probes for screening or assaying a tissue or cell sample are preferably of sufficient length to specifically hybridize only to appropriate, complementary genes or transcripts. Typically the oligonucleotide probes will be at least about 10, 12, 14, 16, 18, 20 or 25 nucleotides in length. In some cases, longer probes of at least 30, 40, or 50 nucleotides will be desirable.

As used herein, oligonucleotide sequences that are complementary to one or more of the genes or gene fragments described in Tables 2-4 refer to oligonucleotides that are capable of hybridizing under stringent conditions to at least part of the nucleotide sequences of said genes. Such hybridizable oligonucleotides will typically exhibit at least about 75% sequence identity at the nucleotide level to said genes, preferably about 80% or 85% sequence identity or more preferably about 90% or 95% or more sequence identity to said genes (see GeneChip® Expression Analysis Manual, Affymetrix, Rev. 3, which is herein incorporated by reference in its entirety).

The phrase “hybridizing specifically to” or “specifically hybridizes” refers to the binding, duplexing, or hybridizing of a molecule substantially to or only to a particular nucleotide sequence or sequences under stringent conditions when that sequence is present in a complex mixture (e.g., total cellular) DNA or RNA.

As used herein a “probe” is defined as a nucleic acid, capable of binding to a target nucleic acid of complementary sequence through one or more types of chemical bonds, usually through complementary base pairing, usually through hydrogen bond formation. As used herein, a probe may include natural (i.e., A, G, U, C, or T) or modified bases (7-deazaguanosine, inosine, etc.). In addition, the bases in probes may be joined by a linkage other than a phosphodiester bond, so long as it does not interfere with hybridization. Thus, probes may be peptide nucleic acids in which the constituent bases are joined by peptide bonds rather than phosphodiester linkages.

A variety of sequencing reactions known in the art can be used to directly sequence nucleic acids for the presence or the absence of one or more polymorphisms of Tables 5-35. Examples of sequencing reactions include those based on techniques developed by Maxam and Gilbert (1977) or Sanger (1977). It is also contemplated that any of a variety of automated sequencing procedures can be utilized, including sequencing by mass spectrometry (see, e.g. PCT International Publication No. WO 94/16101; Cohen et al., 1996; and Griffin et al., 1993), real-time pyrophosphate sequencing method (Ronaghi et al., 1998; and Permutt et al., 2001) and sequencing by hybridization (see e.g. Drmanac et al., 2002).

Other methods of detecting polymorphisms include methods in which protection from cleavage agents is used to detect mismatched bases in RNA/RNA, DNA/DNA or RNA/DNA heteroduplexes (Myers et al., 1985). In general, the technique of “mismatch cleavage” starts by providing heteroduplexes formed by hybridizing (labeled) RNA or DNA containing a wild-type sequence with potentially mutant RNA or DNA obtained from a sample. The double-stranded duplexes are treated with an agent who cleaves single-stranded regions of the duplex such as which will exist due to basepair mismatches between the control and sample strands. For instance, RNA/DNA duplexes can be treated with RNase and DNA/DNA hybrids treated with S1 nuclease to enzymatically digest the mismatched regions. In other embodiments, either DNA/DNA or RNA/DNA duplexes can be treated with hydroxylamine or osmium tetroxide and with piperidine in order to digest mismatched regions. After digestion of the mismatched regions, the resulting material is then separated by size on denaturing polyacrylamide gels to determine the site of a mutation or SNP (see, for example, Cotton et al., 1988; and Saleeba et al., 1992). In a preferred embodiment, the control DNA or RNA can be labeled for detection.

In still another embodiment, the mismatch cleavage reaction employs one or more proteins that recognize mismatched base pairs in double-stranded DNA (so called “DNA mismatch repair” enzymes) in defined systems for detecting and mapping polymorphisms. For example, the mutY enzyme of E. coli cleaves A at G/A mismatches (Hsu et al., 1994). Other examples include, but are not limited to, the MutHLS enzyme complex of E. coli (Smith and Modrich Proc. 1996) and Cel 1 from the celery (Kulinski et al., 2000) both cleave the DNA at various mismatches. According to an exemplary embodiment, a probe based on a polymorphic site corresponding to a polymorphism of Tables 5-35 is hybridized to a cDNA or other DNA product from a test cell or cells. The duplex is treated with a DNA mismatch repair enzyme, and the cleavage products, if any, can be detected from electrophoresis protocols or the like. See, for example, U.S. Pat. No. 5,459,039. Alternatively, the screen can be performed in vivo following the insertion of the heteroduplexes in an appropriate vector. The whole procedure is known to those ordinary skilled in the art and is referred to as mismatch repair detection (see e.g. Fakhrai-Rad of al., 2004).

In other embodiments, alterations in electrophoretic mobility can be used to identify polymorphisms in a sample. For example, single strand conformation polymorphism (SSCP) analysis can be used to detect differences in electrophoretic mobility between mutant and wild type nucleic acids (Orita et al., 1989; Cotton et al., 1993; and Hayashi 1992). Single-stranded DNA fragments of case and control nucleic acids will be denatured and allowed to renature. The secondary structure of single-stranded nucleic acids varies according to sequence. The resulting alteration in electrophoretic mobility enables the detection of even a single base change. The DNA fragments may be labeled or detected with labeled probes. The sensitivity of the assay may be enhanced by using RNA (rather than DNA), in which the secondary structure is more sensitive to a change in sequence. In a preferred embodiment, the method utilizes heteroduplex analysis to separate double stranded heteroduplex molecules on the basis of changes in electrophoretic mobility (Kee et al., 1991).

In yet another embodiment, the movement of mutant or wild-type fragments in a polyacrylamide gel containing a gradient of denaturant is assayed using denaturing gradient gel electrophoresis (DGGE) (Myers et al., 1985). When DGGE is used as the method of analysis, DNA will be modified to insure that it does not completely denature, for example by adding a GC clamp of approximately 40 by of high-melting GC-rich DNA by PCR. In a further embodiment, a temperature gradient is used in place of a denaturing gradient to identify differences in the mobility of control and sample DNA (Rosenbaum et al., 1987). In another embodiment, the mutant fragment is detected using denaturing HPLC (see e.g. Hoogendoorn et al., 2000).

Examples of other techniques for detecting polymorphisms include, but are not limited to, selective oligonucleotide hybridization, selective amplification, selective primer extension, selective ligation, single-base extension, selective termination of extension or invasive cleavage assay. For example, oligonucleotide primers may be prepared in which the polymorphism is placed centrally and then hybridized to target DNA under conditions which permit hybridization only if a perfect match is found (Saiki et al., 1986; Saiki et al., 1989). Such oligonucleotides are hybridized to PCR amplified target DNA or a number of different mutations when the oligonucleotides are attached to the hybridizing membrane and hybridized with labeled target DNA. Alternatively, the amplification, the allele-specific hybridization and the detection can be done in a single assay following the principle of the 5′ nuclease assay (e.g. see Livak et al., 1995). For example, the associated allele, a particular allele of a polymorphic locus, or the like is amplified by PCR in the presence of both allele-specific oligonucleotides, each specific for one or the other allele. Each probe has a different fluorescent dye at the 5′ end and a quencher at the 3′ end. During PCR, if one or the other or both allele-specific oligonucleotides are hybridized to the template, the Taq polymerase via its 5′ exonuclease activity will release the corresponding dyes. The latter will thus reveal the genotype of the amplified product.

Hybridization assays may also be carried out with a temperature gradient following the principle of dynamic allele-specific hybridization or like e.g. Jobs et al., (2003); and Bourgeois and Labuda, (2004). For example, the hybridization is done using one of the two allele-specific oligonucleotides labeled with a fluorescent dye, and an intercalating quencher under a gradually increasing temperature. At low temperature, the probe is hybridized to both the mismatched and full-matched template. The probe melts at a lower temperature when hybridized to the template with a mismatch. The release of the probe is captured by an emission of the fluorescent dye, away from the quencher. The probe melts at a higher temperature when hybridized to the template with no mismatch. The temperature-dependent fluorescence signals therefore indicate the absence or presence of an associated allele, a particular allele of a polymorphic locus, or the like (e.g. Jobs et al., 2003). Alternatively, the hybridization is done under a gradually decreasing temperature. In this case, both allele-specific oligonucleotides are hybridized to the template competitively. At high temperature none of the two probes are hybridized. Once the optimal temperature of the full-matched probe is reached, it hybridizes and leaves no target for the mismatched probe (e.g. Bourgeois and Labuda, 2004). In the latter case, if the allele-specific probes are differently labeled, then they are hybridized to a single PCR-amplified target. If the probes are labeled with the same dye, then the probe cocktail is hybridized twice to identical templates with only one labeled probe, different in the two cocktails, in the presence of the unlabeled competitive probe.

Alternatively, allele specific amplification technology that depends on selective PCR amplification may be used in conjunction with the present invention. Oligonucleotides used as primers for specific amplification may carry the associated allele, a particular allele of a polymorphic locus, or the like, also referred to as “mutation” of interest in the center of the molecule, so that amplification depends on differential hybridization (Gibbs et al., 1989) or at the extreme 3′ end of one primer where, under appropriate conditions, mismatch can prevent, or reduce polymerase extension (Prossner, 1993). In addition it may be desirable to introduce a novel restriction site in the region of the mutation to create cleavage-based detection (Gasparini et al., 1992). It is anticipated that in certain embodiments, amplification may also be performed using Taq ligase for amplification (Barany, 1991). In such cases, ligation will occur only if there is a perfect match at the 3′ end of the 5′ sequence making it possible to detect the presence of a known associated allele, a particular allele of a polymorphic locus, or the like at a specific site by looking for the presence or absence of amplification. The products of such an oligonucleotide ligation assay can also be detected by means of gel electrophoresis. Furthermore, the oligonucleotides may contain universal tags used in PCR amplification and zip code tags that are different for each allele. The zip code tags are used to isolate a specific, labeled oligonucleotide that may contain a mobility modifier (e.g. Grossman et al., 1994).

In yet another alternative, allele-specific elongation followed by ligation will form a template for PCR amplification. In such cases, elongation will occur only if there is a perfect match at the 3′ end of the allele-specific oligonucleotide using a DNA polymerase. This reaction is performed directly on the genomic DNA and the extension/ligation products are amplified by PCR. To this end, the oligonucleotides contain universal tags allowing amplification at a high multiplex level and a zip code for SNP identification. The PCR tags are designed in such a way that the two alleles of a SNP are amplified by different forward primers, each having a different dye. The zip code tags are the same for both alleles of a given SNPs and they are used for hybridization of the PCR-amplified products to oligonucleotides bound to a solid support, chip, bead array or like. For an example of the procedure, see Fan et al. (Cold Spring Harbor Symposia on Quantitative Biology, Vol. LXVIII, pp. 69-78 2003).

Another alternative includes the single-base extension/ligation assay using a molecular inversion probe, consisting of a single, long oligonucleotide (see e.g. Hardenbol et al., 2003). In such an embodiment, the oligonucleotide hybridizes on both side of the SNP locus directly on the genomic DNA, leaving a one-base gap at the SNP locus. The gap-filling, one-base extension/ligation is performed in four tubes, each having a different dNTP. Following this reaction, the oligonucleotide is circularized whereas unreactive, linear oligonucleotides are degraded using an exonuclease such as exonuclease I of E. coli. The circular oligonucleotides are then linearized and the products are amplified and labeled using universal tags on the oligonucleotides. The original oligonucleotide also contains a SNP-specific zip code allowing hybridization to oligonucleotides bound to a solid support, chip, and bead array or like. This reaction can be performed at a high multiplexed level.

In another alternative, the associated allele, a particular allele of a polymorphic locus, or the like is scored by single-base extension (see e.g. U.S. Pat. No. 5,888,819). The template is first amplified by PCR. The extension oligonucleotide is then hybridized next to the SNP locus and the extension reaction is performed using a thermostable polymerase such as ThermoSequenase (GE Healthcare) in the presence of labeled ddNTPs. This reaction can therefore be cycled several times. The identity of the labeled ddNTP incorporated will reveal the genotype at the SNP locus. The labeled products can be detected by means of gel electrophoresis, fluorescence polarization (e.g. Chen et al., 1999) or by hybridization to oligonucleotides bound to a solid support, chip, and bead array or like. In the latter case, the extension oligonucleotide will contain a SNP-specific zip code tag.

In yet another alternative, a SNP is scored by selective termination of extension. The template is first amplified by PCR and the extension oligonucleotide hybridizes in the vicinity of the SNP locus, close to but not necessarily adjacent to it. The extension reaction is carried out using a thermostable polymerase such as ThermoSequenase (GE Healthcare) in the presence of a mix of dNTPs and at least one ddNTP. The latter has to terminate the extension at one of the allele of the interrogated SNP, but not both such that the two alleles will generate extension products of different sizes. The extension product can then be detected by means of gel electrophoresis, in which case the extension products need to be labeled, or by mass spectrometry (see e.g. Storm et al., 2003).

In another alternative, SNPs are detected using an invasive cleavage assay (see U.S. Pat. No. 6,090,543). There are five oligonucleotides per SNP to interrogate but these are used in a two step-reaction. During the primary reaction, three of the designed oligonucleotides are first hybridized directly to the genomic DNA. One of them is locus-specific and hybridizes up to the SNP locus (the pairing of the 3′ base at the SNP locus is not necessary). There are two allele-specific oligonucleotides that hybridize in tandem to the locus-specific probe but also contain a 5′ flap that is specific for each allele of the SNP. Depending upon hybridization of the allele-specific oligonucleotides at the base of the SNP locus, this creates a structure that is recognized by a cleavase enzyme (U.S. Pat. No. 6,090,606) and the allele-specific flap is released. During the secondary reaction, the flap fragments hybridize to a specific cassette to recreate the same structure as above except that the cleavage will release a small DNA fragment labeled with a fluorescent dye that can be detected using regular fluorescence detector. In the cassette, the emission of the dye is inhibited by a quencher.

Methods to Identify Agents that Modulate the Expression of a Nucleic Acid Encoding a Gene Involved in Schizophrenia

The present invention provides methods for identifying agents that modulate the expression of a nucleic acid encoding a gene from Tables 2-4: Such methods may utilize any available means of monitoring for changes in the expression level of the nucleic acids of the invention. As used herein, an agent is said to modulate the expression of a nucleic acid of the invention if it is capable of up- or down-regulating expression of the nucleic acid in a cell. Such cells can be obtained from any parts of the body such as the hair, mouth, rectum, scalp, blood, dermis, epidermis, skin cells, cutaneous surfaces, intertrigious areas, genitalia and fluids, vessels and endothelium. Some non-limiting examples of cells that can be used are: brain cells, cells from the reproductive system, muscle cells, nervous cells, blood and vessels cells, T cell, mast cell, lymphocyte, monocyte, macrophage, and epithelial cells.

In one assay format, the expression of a nucleic acid encoding a gene of the invention (see Tables 2-4) in a cell or tissue sample is monitored directly by hybridization to the nucleic acids of the invention. Cell lines or tissues are exposed to the agent to be tested under appropriate conditions and time and total RNA or mRNA is isolated by standard procedures such as those disclosed in Sambrook et al., (1989) Molecular Cloning: A Laboratory Manual, Cold Spring Harbor Laboratory Press).

Probes to detect differences in RNA expression levels between cells exposed to the agent and control cells may be prepared as described above. Hybridization conditions are modified using known methods, such as those described by Sambrook et al., and Ausubel et al., as required for each probe. Hybridization of total cellular RNA or RNA enriched for polyA RNA can be accomplished in any available format. For instance, total cellular RNA or RNA enriched for polyA RNA can be affixed to a solid support and the solid support exposed to at least one probe comprising at least one, or part of one of the sequences of the invention under conditions in which the probe will specifically hybridize. Alternatively, nucleic acid fragments comprising at least one, or part of one of the sequences of the invention can be affixed to a solid support, such as a silicon chip or a porous glass wafer. The chip or wafer can then be exposed to total cellular RNA or polyA RNA from a sample under conditions in which the affixed sequences will specifically hybridize to the RNA. By examining for the ability of a given probe to specifically hybridize to an RNA sample from an untreated cell population and from a cell population exposed to the agent, agents which up or down regulate expression are identified.

Methods to Identify Agents that Modulate the Activity of a Protein Encoded by a Gene Involved in Schizophrenia Disease

The present invention provides methods for identifying agents that modulate at least one activity of the proteins described in Tables 2-4. Such methods may utilize any means of monitoring or detecting the desired activity. As used herein, an agent is said to modulate the expression of a protein of the invention if it is capable of up- or down-regulating expression of the protein in a cell. Such cells can be obtained from any parts of the body such as the hair, mouth, rectum, scalp, blood, dermis, epidermis, skin cells, cutaneous surfaces, intertrigious areas, genitalia and fluids, vessels and endothelium. Some non-limiting examples of cells that can be used are: brain cells, cells from the reproductive system, muscle cells, nervous cells, blood and vessels cells, T cell, mast cell, lymphocyte, monocyte, macrophage, and epithelial cells.

In one format, the specific activity of a protein of the invention, normalized to a standard unit, may be assayed in a cell population that has been exposed to the agent to be tested and compared to an unexposed control cell population. Cell lines or populations are exposed to the agent to be tested under appropriate conditions and times. Cellular lysates may be prepared from the exposed cell line or population and a control, unexposed cell line or population. The cellular lysates are then analyzed with a probe, such as an antibody probe.

Antibody probes can be prepared by immunizing suitable mammalian hosts utilizing appropriate immunization protocols using the proteins of the invention or antigen-containing fragments thereof. To enhance immunogenicity, these proteins or fragments can be conjugated to suitable carriers. Methods for preparing immunogenic conjugates with carriers such as BSA, KLH or other carrier proteins are well known in the art. In some circumstances, direct conjugation using, for example, carbodiimide reagents may be effective; in other instances linking reagents such as those supplied by Pierce Chemical Co. (Rockford, Ill.) may be desirable to provide accessibility to the hapten. The hapten peptides can be extended at either the amino or carboxy terminus with a cysteine residue or interspersed with cysteine residues, for example, to facilitate linking to a carrier. Administration of the immunogens is conducted generally by injection over a suitable time period and with use of suitable adjuvants, as is generally understood in the art. During the immunization schedule, titers of antibodies are taken to determine adequacy of antibody formation. While the polyclonal antisera produced in this way may be satisfactory for some applications, for pharmaceutical compositions, use of monoclonal preparations is preferred. Immortalized cell lines which secrete the desired monoclonal antibodies may be prepared using standard methods, see e.g., Kohler & Milstein (1992) or modifications which affect immortalization of lymphocytes or spleen cells, as is generally known. The immortalized cell lines secreting the desired antibodies can be screened by immunoassay in which the antigen is the peptide hapten, polypeptide or protein. When the appropriate immortalized cell culture secreting the desired antibody is identified, the cells can be cultured either in vitro or by production in ascites fluid. The desired monoclonal antibodies may be recovered from the culture supernatant or from the ascites supernatant. Fragments of the monoclonal antibodies or the polyclonal antisera which contain the immunologically significant portion(s) can be used as antagonists, as well as the intact antibodies. Use of immunologically reactive fragments, such as Fab or Fab' fragments, is often preferable, especially in a therapeutic context, as these fragments are generally less immunogenic than the whole immunoglobulin. The antibodies or fragments may also be produced, using current technology, by recombinant means. Antibody regions that bind specifically to the desired regions of the protein can also be produced in the context of chimeras derived from multiple species. Antibody regions that bind specifically to the desired regions of the protein can also be produced in the context of chimeras from multiple species, for instance, humanized antibodies. The antibody can therefore be a humanized antibody or a human antibody, as described in U.S. Pat. No. 5,585,089 or Riechmann et al. (1988).

Agents that are assayed in the above method can be randomly selected or rationally selected or designed. As used herein, an agent is said to be randomly selected when the agent is chosen randomly without considering the specific sequences involved in the association of the protein of the invention alone or with its associated substrates, binding partners, etc. An example of randomly selected agents is the use of a chemical library or a peptide combinatorial library, or a growth broth of an organism. As used herein, an agent is said to be rationally selected or designed when the agent is chosen on a non-random basis which takes into account the sequence of the target site or its conformation in connection with the agent's action. Agents can be rationally selected or rationally designed by utilizing the peptide sequences that make up these sites. For example, a rationally selected peptide agent can be a peptide whose amino acid sequence is identical to or a derivative of any functional consensus site. The agents of the present invention can be, as examples, oligonucleotides, antisense polynucleotides, interfering RNA, peptides, peptide mimetics, antibodies, antibody fragments, small molecules, vitamin derivatives, as well as carbohydrates. Peptide agents of the invention can be prepared using standard solid phase (or solution phase) peptide synthesis methods, as is known in the art. In addition, the DNA encoding these peptides may be synthesized using commercially available oligonucleotide synthesis instrumentation and produced recombinantly using standard recombinant production systems. The production using solid phase peptide synthesis is necessitated if non-gene-encoded amino acids are to be included.

Another class of agents of the present invention includes antibodies or fragments thereof that bind to a protein encoded by a gene in Tables 2-4. Antibody agents can be obtained by immunization of suitable mammalian subjects with peptides, containing as antigenic regions, those portions of the protein intended to be targeted by the antibodies (see section above of antibodies as probes for standard antibody preparation methodologies).

In yet another class of agents, the present invention includes peptide mimetics that mimic the three-dimensional structure of the protein encoded by a gene from Tables 2-4. Such peptide mimetics may have significant advantages over naturally occurring peptides, including, for example: more economical production, greater chemical stability, enhanced pharmacological properties (half-life, absorption, potency, efficacy, etc.), altered specificity (e.g., a broad-spectrum of biological activities), reduced antigenicity and others. In one form, mimetics are peptide-containing molecules that mimic elements of protein secondary structure. The underlying rationale behind the use of peptide mimetics is that the peptide backbone of proteins exists chiefly to orient amino acid side chains in such a way as to facilitate molecular interactions, such as those of antibody and antigen. A peptide mimetic is expected to permit molecular interactions similar to the natural molecule. In another form, peptide analogs are commonly used in the pharmaceutical industry as non-peptide drugs with properties analogous to those of the template peptide. These types of non-peptide compounds are also referred to as peptide mimetics or peptidomimetics (Fauchere, 1986; Veber & Freidinger, 1985; Evans et al., 1987) which are usually developed with the aid of computerized molecular modeling. Peptide mimetics that are structurally similar to therapeutically useful peptides may be used to produce an equivalent therapeutic or prophylactic effect. Generally, peptide mimetics are structurally similar to a paradigm polypeptide (i.e., a polypeptide that has a biochemical property or pharmacological activity), but have one or more peptide linkages optionally replaced by a linkage using methods known in the art. Labeling of peptide mimetics usually involves covalent attachment of one or more labels, directly or through a spacer (e.g., an amide group), to non-interfering position(s) on the peptide mimetic that are predicted by quantitative structure-activity data and molecular modeling. Such non-interfering positions generally are positions that do not form direct contacts with the macromolecule(s) to which the peptide mimetic binds to produce the therapeutic effect. Derivitization (e.g., labeling) of peptide mimetics should not substantially interfere with the desired biological or pharmacological activity of the peptide mimetic. The use of peptide mimetics can be enhanced through the use of combinatorial chemistry to create drug libraries. The design of peptide mimetics can be aided by identifying amino acid mutations that increase or decrease binding of the protein to its binding partners. Approaches that can be used include the yeast two hybrid method (see Chien et al., 1991) and the phage display method. The two hybrid method detects protein-protein interactions in yeast (Fields et al., 1989). The phage display method detects the interaction between an immobilized protein and a protein that is expressed on the surface of phages such as lambda and M13 (Amberg et al., 1993; Hogrefe et al., 1993). These methods allow positive and negative selection for protein-protein interactions and the identification of the sequences that determine these interactions.

### Method to Diagnose Schizophrenia

The present invention also relates to methods for diagnosing SCHIZOPHRENIA or a related disease, preferably a subtype of SCHIZOPHRENIA, a predisposition to such a disease and/or disease progression. In some methods, the steps comprise contacting a target sample with (a) nucleic acid molecule(s) or fragments thereof and comparing the concentration of individual mRNA(s) with the concentration of the corresponding mRNA(s) from at least one healthy donor. An aberrant (increased or decreased) mRNA level of at least one gene from Tables 2-4, at least 5 or 10 genes from Tables 2-4, at least 50 genes from Tables 2-4, at least 100 genes from Tables 2-4 or at least 200 genes from Tables 2-4 determined in the sample in comparison to the control sample is an indication of SCHIZOPHRENIA disease or a related subtype or a disposition to such kinds of diseases. For diagnosis, samples are, preferably, obtained from any parts of the body such as the hair, mouth, rectum, scalp, blood, dermis, epidermis, skin cells, cutaneous surfaces, intertrigious areas, genitalia and fluids, vessels and endothelium. Some non-limiting examples of cells that can be used are: brain cells, cells from the reproductive system, muscle cells, nervous cells, blood and vessels cells, T cell, mast cell, lymphocyte, monocyte, macrophage, and epithelial cells.

For analysis of gene expression, total RNA is obtained from cells according to standard procedures and, preferably, reverse-transcribed. Preferably, a DNAse treatment (in order to get rid of contaminating genomic DNA) is performed.

The nucleic acid molecule or fragment is typically a nucleic acid probe for hybridization or a primer for PCR. The person skilled in the art is in a position to design suitable nucleic acids probes based on the information provided in the Tables of the present invention. The target cellular component, i.e. mRNA, e.g., in brain tissue, may be detected directly in situ, e.g. by in situ hybridization or it may be isolated from other cell components by common methods known to those skilled in the art before contacting with a probe. Detection methods include Northern blot analysis, RNase protection, in situ methods, e.g. in situ hybridization, in vitro amplification methods (PCR, LCR, QRNA replicase or RNA-transcription/amplification (TAS, 3SR), reverse dot blot disclosed in EP-B10237362) and other detection assays that are known to those skilled in the art. Products obtained by in vitro amplification can be detected according to established methods, e.g. by separating the products on agarose or polyacrylamide gels and by subsequent staining with ethidium bromide or any other dye or reagent. Alternatively, the amplified products can be detected by using labeled primers for amplification or labeled dNTPs. Preferably, detection is based on a microarray.

The probes (or primers) (or, alternatively, the reverse-transcribed sample mRNAs) can be detectably labeled, for example, with a radioisotope, a bioluminescent compound, a chemiluminescent compound, a fluorescent compound, a metal chelate, or an enzyme.

The present invention also relates to the use of the nucleic acid molecules or fragments described above for the preparation of a diagnostic composition for the diagnosis of SCHIZOPHRENIA or a subtype or predisposition to such a disease.

The present invention also relates to the use of the nucleic acid molecules of the present invention for the isolation or development of a compound which is useful for therapy of SCHIZOPHRENIA. For example, the nucleic acid molecules of the invention and the data obtained using said nucleic acid molecules for diagnosis of SCHIZOPHRENIA might allow for the identification of further genes which are specifically dysregulated, and thus may be considered as potential targets for therapeutic interventions. Furthermore, such diagnostic might also be used for selection of patients that might respond positively or negatively to a potential target for therapeutic interventions (as for the pharmacogenomics and personalized medicine concept well know in the art; see prognostic assays text below).

The invention further provides prognostic assays that can be used to identify subjects having or at risk of developing SCHIZOPHRENIA. In such method, a test sample is obtained from a subject and the amount and/or concentration of the nucleic acid described in Tables 2-4 is determined; wherein the presence of an associated allele, a particular allele of a polymorphic locus, or the likes in the nucleic acids sequences of this invention (see SEQ ID from Tables 5-35) can be diagnostic for a subject having or at risk of developing SCHIZOPHRENIA. As used herein, a “test sample” refers to a biological sample obtained from a subject of interest. For example, a test sample can be a biological fluid, a cell sample, or tissue. A biological fluid can be, but is not limited to saliva, serum, mucus, urine, stools, spermatozoids, vaginal secretions, lymph, amiotic liquid, pleural liquid and tears. Cells can be, but are not limited to: brain cells, cells from the reproductive system, hair cells, muscle cells, nervous cells, blood and vessels cells, dermis, epidermis and other skin cells.

Furthermore, the prognostic assays described herein can be used to determine whether a subject can be administered an agent (e.g., an agonist, antagonist, peptidomimetic, polypeptide, nucleic acid such as antisense DNA or interfering RNA (RNAi), small molecule or other drug candidate) to treat SCHIZOPHRENIA. Specifically, these assays can be used to predict whether an individual will have an efficacious response or will experience adverse events in response to such an agent. For example, such methods can be used to determine whether a subject can be effectively treated with an agent that modulates the expression and/or activity of a gene from Tables 2-4 or the nucleic acids described herein. In another example, an association study may be performed to identify polymorphisms from Tables 5-35 that are associated with a given response to the agent, e.g., an efficacious response or the likelihood of one or more adverse events. Thus, one embodiment of the present invention provides methods for determining whether a subject can be effectively treated with an agent for a disease associated with aberrant expression or activity of a gene from Tables 2-4 in which a test sample is obtained and nucleic acids or polypeptides from Tables 2-4 are detected (e.g., wherein the presence of a particular level of expression of a gene from Tables 2-4 or a particular allelic variant of such gene, such as polymorphisms from Tables 5-35 is diagnostic for a subject that can be administered an agent to treat a disorder such as SCHIZOPHRENIA). In one embodiment, the method includes obtaining a sample from a subject suspected of having SCHIZOPHRENIA or an affected individual and exposing such sample to an agent. The expression and/or activity of the nucleic acids and/or genes of the invention are monitored before and after treatment with such agent to assess the effect of such agent. After analysis of the expression values, one skilled in the art can determine whether such agent can effectively treat such subject. In another embodiment, the method includes obtaining a sample from a subject having or susceptible to developing SCHIZOPHRENIA and determining the allelic constitution of polymorphisms from Tables 5-35 that are associated with a particular response to an agent. After analysis of the allelic constitution of the individual at the associated polymorphisms, one skilled in the art can determine whether such agent can effectively treat such subject.

The methods of the invention can also be used to detect genetic alterations in a gene from Tables 2-4, thereby determining if a subject with the lesioned gene is at risk for a disease associated with SCHIZOPHRENIA. In preferred embodiments, the methods include detecting, in a sample of cells from the subject, the presence or absence of a genetic alteration characterized by at least one alteration linked to or affecting the integrity of a gene from Tables 2-4 encoding a polypeptide or the misexpression of such gene. For example, such genetic alterations can be detected by ascertaining the existence of at least one of: (1) a deletion of one or more nucleotides from a gene from Tables 2-4; (2) an addition of one or more nucleotides to a gene from Tables 2-4; (3) a substitution of one or more nucleotides of a gene from Tables 2-4; (4) a chromosomal rearrangement of a gene from Tables 2-4; (5) an alteration in the level of a messenger RNA transcript of a gene from Tables 2-4; (6) aberrant modification of a gene from Tables 2-4, such as of the methylation pattern of the genomic DNA, (7) the presence of a non-wild type splicing pattern of a messenger RNA transcript of a gene from Tables 2-4; (8) inappropriate post-translational modification of a polypeptide encoded by a gene from Tables 2-4; and (9) alternative promoter use. As described herein, there are a large number of assay techniques known in the art which can be used for detecting alterations in a gene from Tables 2-4. A preferred biological sample is a peripheral blood sample obtained by conventional means from a subject. Another preferred biological sample is a buccal swab. Other biological samples can be, but are not limited to, urine, stools, vaginal secretions, lymph, amiotic liquid, pleural liquid and tears.

In certain embodiments, detection of the alteration involves the use of a probe/primer in a polymerase chain reaction (PCR) (see, e.g., U.S. Pat. Nos. 4,683,195 and 4,683,202), such as anchor PCR or RACE PCR, or alternatively, in a ligation chain reaction (LCR) (see, e.g., Landegran of al., 1988; and Nakazawa et al., 1994), the latter of which can be particularly useful for detecting point mutations in a gene from Tables 2-4 (see Abavaya et al., 1995). This method can include the steps of collecting a sample of cells from a patient, isolating nucleic acid (e.g., genomic DNA, mRNA, or both) from the cells of the sample, contacting the nucleic acid sample with one or more primers which specifically hybridize to a gene from Tables 2-4 under conditions such that hybridization and amplification of the nucleic acid from Tables 2-4 (if present) occurs, and detecting the presence or absence of an amplification product, or detecting the size of the amplification product and comparing the length to a control sample. PCR and/or LCR may be desirable to use as a preliminary amplification step in conjunction with some of the techniques used for detecting a mutation, an associated allele, a particular allele of a polymorphic locus, or the like described in the above sections. Other mutation detection and mapping methods are described in previous sections of the detailed description of the present invention.

The present invention also relates to further methods for diagnosing SCHIZOPHRENIA or a related disorder or subtype, a predisposition to such a disorder and/or disorder progression. In some methods, the steps comprise contacting a target sample with (a) nucleic molecule(s) or fragments thereof and determining the presence or absence of a particular allele of a polymorphism that confers a disorder-related phenotype (e.g., predisposition to such a disorder and/or disorder progression). The presence of at least one allele from Tables 5-35 that is associated with SCHIZOPHRENIA (“associated allele”), at least 5 or 10 associated alleles from Tables 5-35, at least 50 associated alleles from Tables 5-35 at least 100 associated alleles from Tables 5-35, or at least 200 associated alleles from Tables 5-35 determined in the sample is an indication of SCHIZOPHRENIA disease or a related disorder, a disposition or predisposition to such kinds of disorders, or a prognosis for such disorder progression. Such samples and cells can be obtained from any parts of the body such as the hair, mouth, rectum, scalp, blood, dermis, epidermis, skin cells, cutaneous surfaces, intertrigious areas, genitalia and fluids, vessels and endothelium. Some non-limiting examples of cells that can be used are: brain cells, cells from the reproductive system, muscle cells, nervous cells, blood and vessels cells, T cell, mast cell, lymphocyte, monocyte, macrophage, and epithelial cells.

In other embodiments, alterations in a gene from Tables 2-4 can be identified by hybridizing sample and control nucleic acids, e.g., DNA or RNA, to high density arrays or bead arrays containing tens to thousands of oligonucleotide probes (Cronin et al., 1996; Kozal et al., 1996). For example, alterations in a gene from Tables 2-4 can be identified in two dimensional arrays containing light-generated DNA probes as described in Cronin et al., (1996). Briefly, a first hybridization array of probes can be used to scan through long stretches of DNA in a sample and control to identify base changes between the sequences by making linear arrays of sequential overlapping probes. This step allows the identification of point mutations, associated alleles, particular alleles of a polymorphic locus, or the like. This step is followed by a second hybridization array that allows the characterization of specific mutations by using smaller, specialized probe arrays complementary to all variants, mutations, alleles detected. Each mutation array is composed of parallel probe sets, one complementary to the wild-type gene and the other complementary to the mutant gene.

In yet another embodiment, any of a variety of sequencing reactions known in the art can be used to directly sequence a gene from Tables 2-4 and detect an associated allele, a particular allele of a polymorphic locus, or the like by comparing the sequence of the sample gene from Tables 2-4 with the corresponding wild-type (control) sequence (see text described in previous sections for various sequencing techniques and other methods of detecting an associated allele, a particular allele of a polymorphic locus, or the likes in a gene from Tables 2-4. Such methods include methods in which protection from cleavage agents is used to detect mismatched bases in RNA/RNA, DNA/DNA or RNA/DNA heteroduplexes (Myers et al., 1985) and alterations in electrophoretic mobility. Examples of other techniques for detecting point mutations, an associated allele, a particular allele of a polymorphic locus, or the like include, but are not limited to, selective oligonucleotide hybridization, selective amplification, selective primer extension, selective ligation, single-base extension, selective termination of extension or invasive cleavage assay.

Other types of markers can also be used for diagnostic purposes. For example, microsatellites can also be useful to detect the genetic predisposition of an individual to a given disorder. Microsatellites consist of short sequence motifs of one or a few nucleotides repeated in tandem. The most common motifs are polynucleotide runs, dinucleotide repeats (particularly the CA repeats) and trinucleotide repeats. However, other types of repeats can also be used. The microsatellites are very useful for genetic mapping because they are highly polymorphic in their length. Microsatellite markers can be typed by various means, including but not limited to DNA fragment sizing, oligonucleotide ligation assay and mass spectrometry. For example, the locus of the microsatellite is amplified by PCR and the size of the PCR fragment will be directly correlated to the length of the microsatellite repeat. The size of the PCR fragment can be detected by regular means of gel electrophoresis. The fragment can be labeled internally during PCR or by using end-labeled oligonucleotides in the PCR reaction (e.g. Mansfield et al., 1996). Alternatively, the size of the PCR fragment is determined by mass spectrometry. In another alternative, an oligonucleotide ligation assay can be performed. The microsatellite locus is first amplified by PCR. Then, different oligonucleotides can be submitted to ligation at the center of the repeat with a set of oligonucleotides covering all the possible lengths of the marker at a given locus (Zirvi et al., 1999). Another example of design of an oligonucleotide assay comprises the ligation of three oligonucleotides; a 5′ oligonucleotide hybridizing to the 5′ flanking sequence, a repeat oligonucleotide of the length of the shortest allele of the marker hybridizing to the repeated region and a set of 3′ oligonucleotides covering all the existing alleles hybridizing to the 3′ flanking sequence and a portion of the repeated region for all the alleles longer than the shortest one. For the shortest allele, the 3′ oligonucleotide exclusively hybridizes to the 3′ flanking sequence (U.S. Pat. No. 6,479,244).

The methods described herein may be performed, for example, by utilizing pre-packaged diagnostic kits comprising at least one probe nucleic acid selected from the SEQ ID of Tables 5-35, or antibody reagent described herein, which may be conveniently used, for example, in a clinical setting to diagnose patient exhibiting symptoms or a family history of a disorder or disorder involving abnormal activity of genes from Tables 2-4.

### Method to Treat an Animal Suspected of Having Schizophrenia

The present invention provides methods of treating a disease associated with SCHIZOPHRENIA disease by expressing in vivo the nucleic acids of at least one gene from Tables 2-4. These nucleic acids can be inserted into any of a number of well-known vectors for the transfection of target cells and organisms as described below. The nucleic acids are transfected into cells, ex vivo or in vivo, through the interaction of the vector and the target cell. The nucleic acids encoding a gene from Tables 2-4, under the control of a promoter, then express the encoded protein, thereby mitigating the effects of absent, partial inactivation, or abnormal expression of a gene from Tables 2-4.

Such gene therapy procedures have been used to correct acquired and inherited genetic defects, cancer, and viral infection in a number of contexts. The ability to express artificial genes in humans facilitates the prevention and/or cure of many important human disorders, including many disorders which are not amenable to treatment by other therapies (for a review of gene therapy procedures, see Anderson, 1992; Nabel & Feigner, 1993; Mitani & Caskey, 1993; Mulligan, 1993; Dillon, 1993; Miller, 1992; Van Brunt, 1998; Vigne, 1995; Kremer & Perricaudet 1995; Doerfler & Bohm 1995; and Yu et al., 1994).

Delivery of the gene or genetic material into the cell is the first critical step in gene therapy treatment of a disorder. A large number of delivery methods are well known to those of skill in the art. Preferably, the nucleic acids are administered for in vivo or ex vivo gene therapy uses. Non-viral vector delivery systems include DNA plasmids, naked nucleic acid, and nucleic acid complexed with a delivery vehicle such as a liposome. Viral vector delivery systems include DNA and RNA viruses, which have either episomal or integrated genomes after delivery to the cell. For a review of gene therapy procedures, see the references included in the above section.

The use of RNA or DNA based viral systems for the delivery of nucleic acids take advantage of highly evolved processes for targeting a virus to specific cells in the body and trafficking the viral payload to the nucleus. Viral vectors can be administered directly to patients (in vivo) or they can be used to treat cells in vitro and the modified cells are administered to patients (ex vivo). Conventional viral based systems for the delivery of nucleic acids could include retroviral, lentivirus, adenoviral, adeno-associated and herpes simplex virus vectors for gene transfer. Viral vectors are currently the most efficient and versatile method of gene transfer in target cells and tissues. Integration in the host genome is possible with the retrovirus, lentivirus, and adeno-associated virus gene transfer methods, often resulting in long term expression of the inserted transgene. Additionally, high transduction efficiencies have been observed in many different cell types and target tissues.

The tropism of a retrovirus can be altered by incorporating foreign envelope proteins, expanding the potential target population of target cells. Lentiviral vectors are retroviral vectors that are able to transduce or infect non-dividing cells and typically produce high viral titers. Selection of a retroviral gene transfer system would therefore depend on the target tissue. Retroviral vectors are comprised of cis-acting long terminal repeats with packaging capacity for up to 6-10 kb of foreign sequence. The minimum cis-acting LTRs are sufficient for replication and packaging of the vectors, which are then used to integrate the therapeutic gene into the target cell to provide permanent transgene expression. Widely used retroviral vectors include those based upon murine leukemia virus (MuLV), gibbon ape leukemia virus (GaLV), Simian Immuno deficiency virus (SIV), human immuno deficiency virus (HIV), and combinations thereof (see, e.g., Buchscher et al., 1992; Johann et al., 1992; Sommerfelt et al., 1990; Wilson et al., 1989; Miller et al., 1999; and PCT/US94/05700).

In applications where transient expression of the nucleic acid is preferred, adenoviral based systems are typically used. Adenoviral based vectors are capable of very high transduction efficiency in many cell types and do not require cell division. With such vectors, high titer and levels of expression have been obtained. This vector can be produced in large quantities in a relatively simple system. Adeno-associated virus (“AAV”) vectors are also used to transduce cells with target nucleic acids, e.g., in the in vitro production of nucleic acids and peptides, and for in vivo and ex vivo gene therapy procedures (see, e.g., West et al., 1987; U.S. Pat. No. 4,797,368; WO 93/24641; Kotin, 1994; Muzyczka, 1994). Construction of recombinant AAV vectors is described in a number of publications, including U.S. Pat. No. 5,173,414; Tratschin et al., 1985; Tratschin, et al., 1984; Hermonat & Muzyczka, 1984; and Samulski et al., 1989.

In particular, numerous viral vector approaches are currently available for gene transfer in clinical trials, with retroviral vectors by far the most frequently used system. All of these viral vectors utilize approaches that involve complementation of defective vectors by genes inserted into helper cell lines to generate the transducing agent. pLASN and MFG-S are examples are retroviral vectors that have been used in clinical trials (Dunbar et al., 1995; Kohn et al., 1995; Malech et al., 1997). PA317/pLASN was the first therapeutic vector used in a gene therapy trial (Blaese et al., 1995). Transduction efficiencies of 50% or greater have been observed for MFG-S packaged vectors (Ellem et al., 1997; and Dranoff et al., 1997).

Recombinant adeno-associated virus vectors (rAAV) are a promising alternative gene delivery systems based on the defective and nonpathogenic parvovirus adeno-associated type 2 virus. All vectors are derived from a plasmid that retains only the AAV 145 by inverted terminal repeats flanking the transgene expression cassette. Efficient gene transfer and stable transgene delivery due to integration into the genomes of the transduced cell are key features for this vector system (Wagner et al., 1998, Kearns et al., 1996).

Replication-deficient recombinant adenoviral vectors (Ad) are predominantly used in transient expression gene therapy; because they can be produced at high titer and they readily infect a number of different cell types. Most adenovirus vectors are engineered such that a transgene replaces the Ad E1a, E1b, and E3 genes; subsequently the replication defector vector is propagated in human 293 cells that supply the deleted gene function in trans. Ad vectors can transduce multiple types of tissues in vivo, including nondividing, differentiated cells such as those found in the liver, kidney and muscle tissues. Conventional Ad vectors have a large carrying capacity. An example of the use of an Ad vector in a clinical trial involved polynucleotide therapy for antitumor immunization with intramuscular injection (Sterman et al., 1998). Additional examples of the use of adenovirus vectors for gene transfer in clinical trials include Rosenecker et al., 1996; Sterman et al., 1998; Welsh et al., 1995; Alvarez et al., 1997; Topf et al., 1998.

Packaging cells are used to form virus particles that are capable of infecting a host cell. Such cells include 293 cells, which package adenovirus, and w2 cells or PA317 cells, which package retrovirus. Viral vectors used in gene therapy are usually generated by a producer cell line that packages a nucleic acid vector into a viral particle. The vectors typically contain the minimal viral sequences required for packaging and subsequent integration into a host, other viral sequences being replaced by an expression cassette for the protein to be expressed. The missing viral functions are supplied in trans by the packaging cell line. For example, AAV vectors used in gene therapy typically only possess ITR sequences from the AAV genome which are required for packaging and integration into the host genome. Viral DNA is packaged in a cell line, which contains a helper plasmid encoding the other AAV genes, namely rep and cap, but lacking ITR sequences. The cell line is also infected with adenovirus as a helper. The helper virus promotes replication of the AAV vector and expression of AAV genes from the helper plasmid. The helper plasmid is not packaged in significant amounts due to a lack of ITR sequences. Contamination with adenovirus can be reduced by, e.g., heat treatment to which adenovirus is more sensitive than AAV.

In many gene therapy applications, it is desirable that the gene therapy vector be delivered with a high degree of specificity to a particular tissue type. A viral vector is typically modified to have specificity for a given cell type by expressing a ligand as a fusion protein with a viral coat protein on the viruses outer surface. The ligand is chosen to have affinity for a receptor known to be present on the cell type of interest. For example, Han et al., 1995, reported that Moloney murine leukemia virus can be modified to express human heregulin fused to gp70, and the recombinant virus infects certain human breast cancer cells expressing human epidermal growth factor receptor. This principle can be extended to other pairs of viruses expressing a ligand fusion protein and target cells expressing a receptor. For example, filamentous phage can be engineered to display antibody fragments (e.g., Fab or Fv) having specific binding affinity for virtually any chosen cellular receptor. Although the above description applies primarily to viral vectors, the same principles can be applied to nonviral vectors. Such vectors can be engineered to contain specific uptake sequences thought to favor uptake by specific target cells.

Gene therapy vectors can be delivered in vivo by administration to an individual patient, typically by systemic administration (e.g., intravenous, intraperitoneal, intramuscular, subdermal, or intracranial infusion) or topical application. Alternatively, vectors can be delivered to cells ex vivo, such as cells explanted from an individual patient (e.g., lymphocytes, bone marrow aspirates, and tissue biopsy) or universal donor hematopoietic stem cells, followed by reimplantation of the cells into a patient, usually after selection for cells which have incorporated the vector.

Ex vivo cell transfection for diagnostics, research, or for gene therapy (e.g., via re-infusion of the transfected cells into the host organism) is well known to those of skill in the art. In a preferred embodiment, cells are isolated from the subject organism, transfected with a nucleic acid (gene or cDNA), and re-infused back into the subject organism (e.g., patient). Various cell types suitable for ex vivo transfection are well known to those of skill in the art (see, e.g., Freshney et al., 1994; and the references cited therein for a discussion of how to isolate and culture cells from patients).

In one embodiment, stem cells are used in ex vivo procedures for cell transfection and gene therapy. The advantage to using stem cells is that they can be differentiated into other cell types in vitro, or can be introduced into a mammal (such as the donor of the cells) where they will engraft in the bone marrow. Methods for differentiating CD34+ cells in vitro into clinically important immune cell types using cytokines such a GM-CSF, IFN-γ and TNF-α are known (see Inaba et al., 1992).

Stem cells are isolated for transduction and differentiation using known methods. For example, stem cells are isolated from bone marrow cells by panning the bone marrow cells with antibodies which bind unwanted cells, such as CD4+ and CD8+ (T cells), CD45+ (panB cells), GR-1 (granulocytes), and lad (differentiated antigen presenting cells).

Vectors (e.g., retroviruses, adenoviruses, liposomes, etc.) containing therapeutic nucleic acids can be also administered directly to the organism for transduction of cells in vivo. Alternatively, naked DNA can be administered.

Administration is by any of the routes normally used for introducing a molecule into ultimate contact with blood or tissue cells, as described above. The nucleic acids from Tables 2-4 are administered in any suitable manner, preferably with the pharmaceutically acceptable carriers described above. Suitable methods of administering such nucleic acids are available and well known to those of skill in the art, and, although more than one route can be used to administer a particular composition, a particular route can often provide a more immediate and more effective reaction than another route (see Samulski et al., 1989). The present invention is not limited to any method of administering such nucleic acids, but preferentially uses the methods described herein.

The present invention further provides other methods of treating SCHIZOPHRENIA disease such as administering to an individual having SCHIZOPHRENIA disease an effective amount of an agent that regulates the expression, activity or physical state of at least one gene from Tables 2-4. An “effective amount” of an agent is an amount that modulates a level of expression or activity of a gene from Tables 2-4, in a cell in the individual at least about 10%, at least about 20%, at least about 30%, at least about 40%, at least about 50%, at least about 60%, at least about 70%, at least about 80% or more, compared to a level of the respective gene from Tables 2-4 in a cell in the individual in the absence of the compound. The preventive or therapeutic agents of the present invention may be administered, either orally or parenterally, systemically or locally. For example, intravenous injection such as drip infusion, intramuscular injection, intraperitoneal injection, subcutaneous injection, suppositories, intestinal lavage, oral enteric coated tablets, and the like can be selected, and the method of administration may be chosen, as appropriate, depending on the age and the conditions of the patient. The effective dosage is chosen from the range of 0.01 mg to 100 mg per kg of body weight per administration. Alternatively, the dosage in the range of 1 to 1000 mg, preferably 5 to 50 mg per patient may be chosen. The therapeutic efficacy of the treatment may be monitored by observing various parts of the reproductive system and other body parts, or any other monitoring methods known in the art. Other ways of monitoring efficacy can be, but are not limited to monitoring paranoia, depression, hallucinations, or any other SCHIZOPHRENIA related symptom.

The present invention further provides a method of treating an individual clinically diagnosed with SCHIZOPHRENIAs' disease. The methods generally comprises analyzing a biological sample that includes a cell, in some cases, a cell, from an individual clinically diagnosed with SCHIZOPHRENIA disease for the presence of modified levels of expression of at least 1 gene, at least 10 genes, at least 50 genes, at least 100 genes, or at least 200 genes from Tables 2-4. A treatment plan that is most effective for individuals clinically diagnosed as having a condition associated with SCHIZOPHRENIA disease is then selected on the basis of the detected expression of such genes in a cell. Treatment may include administering a composition that includes an agent that modulates the expression or activity of a protein from Tables 2-4 in the cell. Information obtained as described in the methods above can also be used to predict the response of the individual to a particular agent. Thus, the invention further provides a method for predicting a patient's likelihood to respond to a drug treatment for a condition associated with SCHIZOPHRENIA disease, comprising determining whether modified levels of a gene from Tables 2-4 is present in a cell, wherein the presence of protein is predictive of the patient's likelihood to respond to a drug treatment for the condition. Examples of the prevention or improvement of symptoms accompanied by SCHIZOPHRENIA disease that can monitored for effectiveness include prevention or improvement of paranoia, depression, hallucinations, or any other SCHIZOPHRENIA related symptom.

The invention also provides a method of predicting a response to therapy in a subject having SCHIZOPHRENIA disease by determining the presence or absence in the subject of one or more markers associated with SCHIZOPHRENIA disease described in Tables 5-35, diagnosing the subject in which the one or more markers are present as having SCHIZOPHRENIA disease, and predicting a response to a therapy based on the diagnosis e.g., response to therapy may include an efficacious response and/or one or more adverse events. The invention also provides a method of optimizing therapy in a subject having SCHIZOPHRENIA disease by determining the presence or absence in the subject of one or more markers associated with a clinical subtype of SCHIZOPHRENIA disease, diagnosing the subject in which the one or more markers are present as having a particular clinical subtype of SCHIZOPHRENIA disease, and treating the subject having a particular clinical subtype of SCHIZOPHRENIA disease based on the diagnosis. As an example, treatment for the paranoia, depression, hallucinations or any other symptoms from any subtypes of SCHIZOPHRENIA.

Thus, while there are a number of available treatments to relieve the symptoms of SCHIZOPHRENIA, they all are accompanied by various side effects, high costs, and long complicated treatment protocols, which are often not available and effective in a large number of individuals. Symptoms also often come back shortly after treatments are stopped. Accordingly, there remains a need in the art for more effective and otherwise improved methods for diagnosing, treating and preventing SCHIZOPHRENIA. Thus, there is a continuing need in the medical arts for genetic markers of SCHIZOPHRENIA disease and guidance for the use of such markers. The present invention fulfills this need and provides further related advantages.

### EXAMPLES

### Example 1

### Identification of Cases and Controls

All individuals were sampled from the Quebec founder population (QFP). Membership in the founder population was defined as having four grandparents of the affected child having French Canadian family names and being born in the Province of Quebec, Canada or in adjacent areas of the Provinces of New Brunswick and Ontario or in New England or New York State. The Quebec founder population is expected to have two distinct advantages over general populations for LD mapping: 1) increased LD resulting from a limited number of generations since the founding of the population and 2) increased genetic alleic homogeneity because of the restricted number of founders (estited 2600 effective founders, Charbonneau et al., 1987). Reduced allelic heterogeneity will act to increase relative risk imparted by the remaining alleles and so increase the power of case/control studies to detect genes and gene alleles involved in complex disorders within the Quebec population. The specific combination of age in generations, optimal number of founders and large present population size makes the QFP optimal for LD-based gene mapping.

All enrolled QFP subjects (patients and controls) provided a 20 ml blood sample (2 barcoded tubes of 10 ml). Following centrifugation, the buffy coat containing the white blood cells was isolated from each tube. Genomic DNA was extracted from the buffy coat from one of the tubes, and stored at 4° C. until required for genotyping. DNA extraction was performed with a commercial kit using a guanidine hydrochloride based method (FlexiGene, Qiagen) according to the manufacturer's instructions. The extraction method yielded high molecular weight DNA, and the quality of every DNA sample was verified by agarose gel electrophoresis. Genomic DNA appeared on the gel as a large band of very high molecular weight. The remaining two buffy coats were stored at −80° C. as backups.

The QFP samples were collected as cases and controls consisting of Schizophrenia disease subjects and controls. 516 cases and 516 controls were used for the analysis reported here. The cases had a clinicians based diagnosis.

### Example 2

### Genome Wide Association

Genotyping was performed using the QLDM-Max SNP map using Illumina's Infinium-II technology Single Sample Beadchips. The QLDM-Max map contains 374,187 SNPs. The SNPs are contained in the Illumina HumanHap-300 arrays plus two custom SNP sets of approximately 30,000 markers each. The HumanHap-300 chip includes 317,503 tag SNPs derived from the Phase I HapMap data. The additional (approx.) 60,000 SNPs were selected by to optimize the density of the marker map across the genome matching the LD pattern in the Quebec Founder Population, as established from previous studies at Genizon, and to fill gaps in the Illumina HumanHap-300 map. The SNPs were genotyped on the 516 cases and 516 controls for a total of ˜386,160,484 genotypes.

The genotyping information was entered into a Unified Genotype Database (a proprietary database under development) from which it was accessed using custom-built programs for export to the genetic analysis pipeline. Analyses of these genotypes were performed with the statistical tools described in Example 3. The GWS and the different analyses permitted the identification of candidate chromosomal regions linked to Schizophrenia disease (Table 1).

### Example 3

### Genetic Analysis

1. Dataset Quality Assessment

Prior to performing any analysis, the sample was examined to ascertain that no subjects were related more closely than 5 meiotic steps.

The data were then subjected to a cleaning step. The program, DataStats was used to calculate the following statistics per marker or per <individual>:
Minor allele frequency (MAF) for each marker
Number of markers with MAF <5%, <4%, <3%, <2%, <1%
Number of missing values for each marker and individual
Monomorphic markers
Departure from Hardy-Weinberg equilibrium within control individuals for each marker
The following acceptance criteria were required for further analysis:
   Missing values per marker or individual <1%
   Minor allele frequency per marker ≧4%,
   Allele frequencies for controls in Hardy-Weinberg equilibrium
Markers and individuals not meeting criteria were removed from the dataset using DataPullPC. If a case or a control was removed by the cleaning process, its region and gender matched case or control were also removed from the analysis.

2. Phase Determination

Haplotypes will were estimated from the case/control genotype data using ggplem a modified version of the PL-EM algorithm. The programs geno2patctr and tagger determined case and control genotypes and prepared the data in the input format for PL-EM. An EM algorithm module consisting of several applications was used to resolve phase ambiguities. PLEMPre first recoded the genotypes for input into the PL-EM algorithm, which used an 11-marker sliding block for haplotype estimation and deposited the constructed haplotypes into a file, happatctr which was the input file for haplotype association analysis performed by the program, LDSTATS.

The program GeneWriter was used to create a case-control genotype file, genopatctr, which was the input for the program, SINGLETYPE, which was used to perform single marker case-control association analysis.

3. Haplotype Association Analysis

Haplotype association analysis was performed using the program LDSTATS. LDSTATS tests for association of haplotypes with the disease phenotype. The algorithms LDSTATS (v2.0) and LDSTATS (v4.0) define haplotypes using multi-marker windows that advance across the marker map in one-marker increments. Windows of size 1, 3, 5, 7, and 9 were analyzed. At each position the frequency of haplotypes in cases and controls was determined and a chi-square statistic was calculated from case control frequency tables. For LDSTATS v2.0, the significance of the chi-square for single marker and 3-marker windows was calculated as Pearson's chi-square with degrees of freedom. Larger windows of multi-allelic haplotype association were tested using Smith's normalization of the square root of Pearson's Chi-square.

LDSTATS v4.0 calculates significance of chi-square values using a permutation test in which case-control status is randomly permuted until 350 permuted chi-square values are observed that are greater than or equal to chi-square value of the actual data. The P value is then calculated as 350/the number of permutations required.

Tables 5-35 lists the results for association analysis using LDSTATs (v2.0 and v4.0) for the candidate regions described in Table 1 based on the genome wide scan genotype data for the full cohort QFP cases and controls. For each one of these regions, we report in Tables 5-35 the allele frequencies and the relative risk (RR) for the haplotypes contributing to the best signal at each SNP in the region.

4. Singletype Analysis The program SINGLETYPE was used to calculate both allelic and genotype association for each single marker, one at a time using the genotype data in the file, genopatctr as input. Allelic association was tested using a 2×2 contingency table comparing allele 1 in cases and controls and allele 2 in cases and controls and genotype association was tested using a 2×3 contingency table comparing genotype 11 in cases and controls, genotype 12 in cases and controls and genotype 22 in cases and controls. SINGLETYPE was also used to test dominant and recessive models (11 and 12 genotypes combined vs. 22; or 22 and 12 genotypes combined vs. 11).

5. Conditional Analyses

Conditional analyses were performed on subsets of the original set of 486 cases using the program LDSTATS (v2.0). The selection of a subset of cases and their matched controls was based on the carrier status of cases at a gene or locus of interest. We selected genes CIAS1 on chromosome 1, PTPRD on chromosome 9 and SPG3A on chromosome 14 based on our haplotype-based association findings using LDSTAT (v2.0). We selected genes WNT7A on chromosome 3 and PAFAH1B1 on chromosome 17, based on our single SNP-based association findings using LDSTAT (v2.0).

The most significant association in CIAS1, using build 36, was obtained with a haplotype window of size 7 containing SNPs corresponding to SEQ IDs 11974, 11975, 11976, 11977, 11978, 11979, 11980 (see Table below for conversion to the specific DNA alleles used). A reduced haplotype diversity was observed and we selected two sets of risk haplo-genotypes for conditional analyses. The first and more narrowly-defined risk set consisted of haplo-genotypes 1 2 1 1 2 2 2/1 2 1 1 2 2 2, 1 2 1 1 2 2 2/1 2 1 1 2 2 2, 2 2 1 1 2 2 2/1 1 1 1 1 1 1, 2 2 1 1 2 2 2/2 1 1 1 1 1 1, 2 2 1 1 2 2 2/2 2 2 2 1 1 1, 2 2 1 1 2 2 2/2 1 1 1 1 1 2, 2 1 1 1 1 1 1/2 2 2 2 1 1 1. The second set consisted of haplo-genotypes found in the first set augmented with 2 2 2 2 1 1 1/2 2 2 2 1 1 1, 1 1 1 1 1 1 1/2 1 1 1 1 1 1, 1 2 1 1 2 2 2/1 2 1 2 2 1 1, 2 1 1 1 1 1 1/2 1 1 1 1 1 2, 1 2 1 1 1 1 1/2 2 2 2 1 1 1, 1 2 1 2 2 1 1/2 2 1 1 2 2 2, 1 2 1 1 1 1 1/2 2 1 1 2 2 2, 1 1 2 2 1 1 1/2 2 2 2 1 1 1. Using the first risk set, we partitioned the cases into two groups; the first group consisting of those cases that were carrier of a risk haplo-genotype and the second group consisting of the remaining cases, the non-carriers. The resulting sample sizes were respectively 80 and 406. LDSTAT (v2.0) was run in each group and regions showing association with schizophrenia using single SNPs are reported in Table 5.1. Regions associated with schizophrenia in the group of non-carriers (CIAS1-1_cd_not) indicate the existence of risk factors acting independently of CIAS1 (Table 5.2). Using the larger risk set, we partitioned the cases into two groups; the first group consisting of those cases that were carrier of a risk haplo-genotype and the second group consisting of the remaining cases, the non-carriers. The resulting sample sizes were respectively 144 and 342. LDSTAT (v2.0) was run in each group and regions showing association with schizophrenia using haplotypes or using single SNP are reported in Tables 15.1 and 29.1. Regions associated with schizophrenia in the group of carriers (CIAS1-1_cr2_has) indicate the presence of an epistatic interaction between risk factors in those regions and risk factors in CIAS1 (Table 15.2). Regions associated with schizophrenia in the group of non-carriers (CIAS1-1_cr2_not) indicate the existence of risk factors acting independently of CIAS1 (Table 29.2)

A second conditional analysis was performed using gene PTPRD on chromosome 9. The most significant association in PTPRD, using build 36, was obtained with a haplotype window of size 5 containing SNPs corresponding to SEQ IDs 15579, 15580, 15581, 15582, 15583 (see Table below for conversion to the specific DNA alleles used). A reduced haplotype diversity was observed and we selected two sets of risk haplo-genotypes and a set of protective haplotypes for conditional analyses. The first risk set consisted of haplo-genotype 2 1 1 2 1/2 1 1 2 1 while the second set consisted of haplotype 2 1 1 2 1, excluding heterozygote haplo-genotypes 2 1 1 2 1/2 2 1 1 1, 2 1 1 2 1/2 1 2 2 2 and 2 1 1 2 1/2 1 1 1 1 due to dominance considerations. The protective set consisted of haplo-genotypes 2 1 1 2 1/2 1 2 2 2, 2 2 1 1 1/2 2 1 1 1, 2 2 1 1 1/2 1 2 2 2, 2 2 1 1 1/2 1 1 1 1, 2 2 1 1 1/2 1 1 2 2, 2 2 1 1 1/1 1 1 1 1 and 2 1 2 2 2/2 1 2 2 2. Using the first risk set, we partitioned the cases into two groups; the first group consisting of those cases that were carrier of a risk haplo-genotype and the second group consisting of the remaining cases, the non-carriers. The resulting sample sizes were respectively 155 and 331. LDSTAT (v2.0) was run in each group and regions showing association with schizoprenia using single SNPs are reported in Table 34.1 for the group of carriers and in Table 33.1 for the group of non-carriers using all haplotypes. Regions associated with schizophrenia in the group of carriers (PTPRD-1_cd_has) indicate the presence of an epistatic interaction between risk factors in those regions and risk factors in PTPRD (Table 34.2). Regions associated with schizophrenia in the group of non-carriers (PTPRD-1_cr1_not) indicate the existence of risk factors acting independently of PTPRD (Table 33.2). Using the second risk set, we partitioned the cases into two groups; the first group consisting of those cases that were carrier of a risk haplo-genotype and the second group consisting of the remaining cases, the non-carriers. The resulting sample sizes were respectively 250 and 236. LDSTAT (v2.0) was run in each group and regions showing association with schizoprenia using single SNPs are reported in Table 35.1 for the group of carriers and in Table 6.1 for the group of non-carriers using all haplotypes. Regions associated with schizophrenia in the group of carriers (PTPRD-1_cr2_has) indicate the presence of an epistatic interaction between risk factors in those regions and risk factors in PTPRD (Table 35.2). Regions associated with schizophrenia in the group of non-carriers (PTPRD-1_cr2_not) indicate the existence of risk factors acting independently of PTPRD (Table6.2). Using the protective set, we partitioned the cases into two groups; the first group consisting of those cases that were carrier of a risk haplo-genotype and the second group consisting of the remaining cases, the non-carriers. The resulting sample sizes were respectively 96 and 390. LDSTAT (v2.0) was run in each group and regions showing association with schizoprenia using single SNPs and all haplotypes are reported in Table 32.2 for the group of carriers and in Table 31.1 for the group of non-carriers using single SNPs. Regions associated with schizophrenia in the group of carriers (PTPRD-1_cp_has) indicate the existence of risk factors acting independently of PTPRD (Table 32.3). Regions associated with schizophrenia in the group of non-carriers (PTPRD-1_cp_not) indicate the presence of an epistatic interaction between risk factors in those regions and risk factors in PTPRD (Table 31.2).

A third conditional analysis was performed using gene SPG3A on chromosome 14. The most significant association in SPG3A, using build 36, was obtained with a haplotype window of size 9 containing SNPs corresponding to SEQ IDs 17338, 17339, 17340, 17341, 17342, 17343, 17344, 17345, 17346 (see Table below for conversion to the specific DNA alleles used). A reduced haplotype diversity was observed and we selected a set of risk haplo-genotypes and a set of protective haplotypes for conditional analyses. The risk set consisted of haplotypes 2 1 2 1 1 2 2 1 1, 1 2 2 2 1 2 1 2 1, 2 1 1 2 1 1 1 1 2, 2 1 1 2 1 1 1 2 1, 2 1 2 1 2 2 1 1 2, 2 1 1 2 1 1 2 1 1 and 2 1 2 1 1 2 1 1 1, excluding, due to dominance considerations, haplo-genotypes containing allele 2 1 1 2 1 1 1 2 1 with alleles 2 1 2 1 1 1 1 2 1, 2 1 2 1 1 2 1 1 2, 2 1 1 1 1 1 1 2 1, 1 2 2 2 1 2 2 1 1 or 2 1 1 1 1 1 1 1 2, and haplo-genotypes containing allele 2 1 2 1 2 2 1 1 2 with alleles 2 1 2 1 1 1 1 2 1, 2 1 2 1 1 2 1 1 2 or 1 2 2 2 1 2 2 1 1. The protective set consisted of haplo-genotypes 2 1 2 1 1 1 1 2 1/2 1 2 1 1 1 1 2 1, 2 1 2 1 1 1 1 2 1/2 1 2 1 2 2 1 1 2, 2 1 2 1 1 1 1 2 1/2 1 1 1 1 1 1 2 1, 2 1 2 1 1 1 1 2 1/2 1 1 1 1 1 1 1 2, 2 1 2 1 1 2 1 1 2/2 1 1 2 1 1 1 2 1, 2 1 1 2 1 1 1 2 1/2 1 1 1 1 1 1 2 1 and 2 1 1 2 1 1 1 2 1/1 2 2 2 1 2 2 1 1. Using the risk set, we partitioned the cases into two groups; the first group consisting of those cases that were carrier of a risk haplo-genotype and the second group consisting of the remaining cases, the non-carriers. The resulting sample sizes were respectively 134 and 352. LDSTAT (v2.0) was run in each group and regions showing association with schizophrenia using all haplotypes in Table 9.2. Regions associated with schizophrenia in the group of non-carriers (SPG3A-1_cr_not) indicate the existence of risk factors acting independently of SPG3A (Table 9.4). Using the protective set, we partitioned the cases into two groups; the first group consisting of those cases that were carrier of a risk haplo-genotype and the second group consisting of the remaining cases, the non-carriers. The resulting sample sizes were respectively 99 and 387. LDSTAT (v2.0) was run in each group and regions showing association with schizoprenia are reported in Table 8.1 for the group of carriers and in Table 7.1 for the group of non-carriers using single SNPs and all haplotypes. Regions associated with schizophrenia in the group of carriers (SPG3A-1_cp_has) indicate the existence of risk factors acting independently of SPG3A (Table 8.2). Regions associated with schizophrenia in the group of non-carriers (SPG3A-1_cp_not) indicate the presence of an epistatic interaction between risk factors in those regions and risk factors in SPG3A (Table 7.2).

A fourth conditional analysis was performed using gene WNT7A on chromosome 3. The most significant association signal based on single SNPs in WNT7A, using build 36, was obtained with a SNP corresponding to SEQ ID 12686 (see Table below for conversion to the specific DNA alleles used). We selected a risk allele for conditional analyses. The set consisted of allele 2. Using this risk set, we partitioned the cases into two groups; the first group consisting of those cases that were carrier of a risk allele and the second group consisting of the remaining cases, the non-carriers. The resulting sample sizes were respectively 314 and 172. LDSTAT (v2.0) was run in each group and regions showing association with schizophrenia using single SNPs are reported in Table 26.1 for the group of carriers and in Table 30.1 for the group of non-carriers using all haplotypes. Regions associated with schizophrenia in the group of carriers (WNT7A-1_cr_has) indicate the presence of an epistatic interaction between risk factors in the region and risk factors in WNT7A (Table 26.2). Regions associated with schizophrenia in the group of non-carriers (WNT7A-1_cr_not) indicate the existence of risk factors acting independently of WNT7A (Table 30.2).

A fifth conditional analysis was performed using gene PAFAH1B1 on chromosome 17. The most significant association signal based on single SNPs in PAFAH1B1, using build 36, was obtained with a SNP corresponding to SEQ ID 18108 (see Table below for conversion to the specific DNA alleles used). We selected a risk genotype for conditional analyses. The set consisted of genotype 1/1. Using this risk set, we partitioned the cases into two groups; the first group consisting of those cases that were carrier of a risk allele and the second group consisting of the remaining cases, the non-carriers. The resulting sample sizes were respectively 319 and 167. LDSTAT (v2.0) was run in each group and regions showing association with schizoprenia using single SNPs are reported in Table 10.1 for the group of carriers and in Table 11.1 for the group of non-carriers using all haplotypes. Regions associated with schizophrenia in the group of carriers (PAFAH1B1-1_cr_has) indicate the presence of an epistatic interaction between risk factors in the region and risk factors in PAFAH1B1 (Table 10.2). Regions associated with schizophrenia in the group of non-carriers (PAFAH1B1-1_cr_not) indicates the existence of risk factors acting independently of PAFAH1B1 (Table 11.2).

Other conditional analyses were performed on subsets of the original set of 357 schizophrenic cases with paranoia using the program LDSTATS (v2.0). The selection of a subset of cases and their matched controls was based on the carrier status of cases at NRG1 on chromosome 8. The most significant association in NRG1, for the paranoid subset, was obtained with a haplotype window of size 5 containing SNPs corresponding to SEQ IDs 15139, 15140, 15141, 15142, 15143 (see Table below for conversion to the specific DNA alleles used). A reduced haplotype diversity was observed and we selected two sets of risk and two sets of protective haplo-genotypes for conditional analyses. The first and more narrowly-defined risk set consisted of haplo-genotypes 2 1 1 2 1/2 1 2 1 2, 2 1 1 2 1/1 2 2 1 1, 2 1 2 1 2/1 2 2 1 1, 2 1 2 2 2/1 2 2 1 1. The second set consisted of haplo-genotypes 2 1 2 1 2/1 2 2 1 1, 2 1 2 2 2/1 2 2 1 1 and haplotype 2 1 1 2 1, excluding, due to dominance considerations, heterozygote with haplotypes 2 1 2 1 1, 2 1 2 2 2, 2 2 2 1 2 or 2 1 2 2 1. Using the first risk set, we partitioned the cases into two groups; the first group consisting of those cases that were carrier of a risk haplo-genotype and the second group consisting of the remaining cases, the non-carriers. The resulting sample sizes were respectively 177 and 180. LDSTAT (v2.0) was run in each group and regions showing association with schizophrenia are reported in Tables 18.2 and 19.2. Regions associated with schizophrenia in the group of carriers (NRG1-1_cr1_has) indicate the presence of an epistatic interaction between risk factors in those regions and risk factors in NRG1 (Table 18.3). Regions associated with schizophrenia in the group of non-carriers (NRG1-1_crl_not) indicate the existence of risk factors acting independently of NRG1 (Table 19.3). Using the larger risk set, we partitioned the cases into two groups; the first group consisting of those cases that were carrier of a risk haplo-genotype and the second group consisting of the remaining cases, the non-carriers. The resulting sample sizes were respectively 214 and 143. LDSTAT (v2.0) was run in each group and regions showing association with schizophrenia are reported in Tables 20.2 and 21.1. Regions associated with schizophrenia in the group of carriers (NRG1-1_cr2_has) indicate the presence of an epistatic interaction between risk factors in those regions and risk factors in NRG1 (Table 20.3) while regions associated with schizophrenia in the group of non-carriers (NRG1-1_cr2_not) indicate the existence of risk factors acting independently of NRG1 (Table 21.2). The first and more narrowly-defined protective set consisted of haplo-genotypes 2 1 2 1 1/1 2 2 1 1, 1 2 2 1 1/1 2 2 1 1 and 1 2 2 1 1/2 2 2 1 2. The second protective set consisted of haplo-genotypes 1 2 2 1 1/1 2 2 1 1, 1 2 2 1 1/2 2 2 1 2, 1 2 2 1 1/1 2 2 2 2 and haplotype 2 1 2 1 1 excluding heterozygotes with haplotype 2 1 2 1 2. Using the first protective set, we partitioned the cases into two groups; the first group consisting of those cases that were carrier of a protective haplo-genotype and the second group consisting of the remaining cases, the non-carriers. The resulting sample sizes were respectively 103 and 254. LDSTAT (v2.0) was run in each group and regions showing association with schizophrenia are reported in Tables 14.1 and 16.2. Regions associated with schizophrenia in the group of carriers (NRG1-1_cp1_has) indicate the existence of risk factors acting independently of NRG1 (Table 14.2). Regions associated with schizophrenia in the group of non-carriers (NRG1-1_cp1_not) indicate the presence of an epistatic interaction between risk factors in those regions and risk factors in NRG1 (Table 16.3). Using the larger risk set, we partitioned the cases into two groups; the first group consisting of those cases that were carrier of a risk haplo-genotype and the second group consisting of the remaining cases, the non-carriers. The resulting sample sizes were respectively 122 and 235. LDSTAT (v2.0) was run in each group and regions showing association with schizophrenia are reported in Tables 17.2. Regions associated with schizophrenia in the group of non-carriers (NRG1-1_cp2_not) indicate the presence of an epistatic interaction between risk factors in those regions and risk factors in NRG1 (Table 17.3).

For each region that was associated with schizophrenia in the conditional analyses, we report the allele frequency and the relative risk (RR) for each SNP in the region. For a given SNP, the association with schizophrenia was evaluated with a Chi-Square test by comparing the allele frequency in the cases with the allele frequency in the controls. For a given SNP, the association with schizophrenia was evaluated with a Chi-Square test by comparing the allele frequency in the cases with the allele frequency in the controls. Alleles with a relative risk greater than one increase the risk of developing schizophrenia while alleles with a relative risk less than one are protective and decrease the risk.

DNA alleles used in haplotypes (CIAS1)

| SeqID | 11974 | 11975 | 11976 | 11977 | 11978 | 11979 | 11980 |
|---|---|---|---|---|---|---|---|
| Position | 245602706 | 245603769 | 245604311 | 245606936 | 245608675 | 245618707 | 245619172 |
| Alleles | T\|C | T\|G | T\|G | A\|G | T\|C | | |
| 1111111 | T | T | T | A | T | T | T |
| 1122111 | T | T | G | G | T | T | T |
| 1211111 | T | G | T | A | T | T | T |
| 1211222 | T | G | T | A | C | C | C |
| 1212211 | T | G | T | G | C | T | T |
| 2111111 | C | T | T | A | T | T | T |
| 2111112 | C | T | T | A | T | T | C |
| 2211222 | C | G | T | A | C | C | C |
| 2222111 | C | G | G | G | T | T | T |

DNA alleles used in haplotypes (PTPRD)

| SeqID | 15579 | 15580 | 15581 | 15582 | 15583 |
|---|---|---|---|---|---|
| Position | 8464233 | 8465677 | 8467093 | 8469185 | 8470144 |
| Alleles | T\|C | T\|C | A\|C | A\|G | T\|C |
| 11111 | T | T | A | A | T |
| 21111 | C | T | A | A | T |
| 21121 | C | T | A | G | T |
| 21122 | C | T | A | G | C |
| 21222 | C | T | C | G | C |
| 22111 | C | C | A | A | T |

DNA alleles used in haplotypes (SPG3)

| SeqID | 17338 | 17339 | 17340 | 17341 | 17342 | 17343 | 17344 | 17345 | 17346 |
|---|---|---|---|---|---|---|---|---|---|
| Position | 50069943 | 50099463 | 50122718 | 50139753 | 50156137 | 50162892 | 50181639 | 50191450 | 50193977 |
| Alleles | T\|C | A\|G | T\|G | T\|C | A\|G | A\|G | A\|G | A\|G | T\|C |
| 122212121 | T | G | G | C | A | G | A | G | T |
| 122212211 | T | G | G | C | A | G | G | A | T |
| 211111112 | C | A | T | T | A | A | A | A | C |
| 211111121 | C | A | T | T | A | A | A | G | T |
| 211211112 | C | A | T | C | A | A | A | A | C |
| 211211121 | C | A | T | C | A | A | A | G | T |
| 211211211 | C | A | T | C | A | A | G | A | T |
| 212111121 | C | A | G | T | A | A | A | G | T |
| 212112111 | C | A | G | T | A | G | A | A | T |
| 212112112 | C | A | G | T | A | G | A | A | C |
| 212112211 | C | A | G | T | A | G | G | A | T |
| 212122112 | C | A | G | T | G | G | A | A | C |

DNA alleles used in haplotypes (WNT7A)

| SeqID | 12686 |
|---|---|
| Position | 13905134 |
| Alleles | A\|C |
| 1 | A |
| 2 | C |

DNA alleles used in haplotypes (PAFAH1B1)

| SeqID | 18108 |
|---|---|
| Position | 2414919 |
| Alleles | A\|C |
| 1 | A |
| 2 | C |

DNA alleles used in haplotypes (NRG1)

| SeqID | 15139 | 15140 | 15141 | 15142 | 15143 |
|---|---|---|---|---|---|
| Position | 32216518 | 32217872 | 32223600 | 32234798 | 32236954 |
| Alleles | T\|G | A\|C | A\|G | A\|G | T\|C |
| 12211 | T | C | G | A | T |
| 21121 | G | A | A | 6 | T |
| 21211 | G | A | G | A | T |
| 21212 | G | A | G | A | C |
| 21221 | G | A | G | G | T |
| 21222 | G | A | G | G | C |
| 22212 | G | C | G | A | C |
| 12222 | T | C | G | G | C |

6. Phenotype Analyses

The choice of phenotype for complex diseases, such as schizophrenia, can have a large impact on the success of gene discovery. It is quite possible that some genes affect only highly specific forms of a disease. It may be possible to discover specific genes which are obscured within the entire data set through the analysis of specific homogeneous sub-types of the disease. For this purpose we subdivided the entire sample into the following sub-phenotypes:

| Subphenotype | number of case/controls |
|---|---|
| Male cases | 349/349 |
| Female cases | 167/167 |
| Male cases with age of onset <20 years | 118/118 |
| Male cases with age of onset ≧20 years | 231/231 |
| Female cases with age of onset <25 years | 86/86 |
| Female cases with age of onset ≧25 years | 80/80 |
| Paranoid DSM-IV subtype | 380/380 |

A separate whole genome association study WGAS was performed on each sub-phenotype. Genome wide significance of results for each phenotype was tested by two types of permutation. In the first method, case and control status for each pair of cases and controls was randomly permuted. This tests the genome wide significance of the results for the subphenotype analysis. In the second method, subsets of the appropriate size were randomly selected from the entire data set. This tests whether the specific sub-phenotype gives results that are significantly distinct from the analysis of the entire data set.

### Example 4

### Gene Identification and Characterization

A series of gene characterization was performed for each candidate region described in Table 1. Any gene or EST mapping to the interval based on public map data or proprietary map data was considered as a candidate SCHIZOPHRENIA disease gene. The approach used to identify all genes located in the critical regions is described below.

Public Gene Mining

Once regions were identified using the analyses described above, a series of public data mining efforts were undertaken, with the aim of identifying all genes located within the critical intervals as well as their respective structural elements (i.e., promoters and other regulatory elements, UTRs, exons and splice sites). The initial analysis relied on annotation information stored in public databases (e.g. NCBI, UCSC Genome Bioinformatics, Entrez Human Genome Browser, OMIM—see below for database URL information). Tables 2-4 lists the genes that have been mapped to the candidate regions.

For some genes the available public annotation was extensive, whereas for others very little was known about a gene's function. Customized analysis was therefore performed to characterize genes that corresponded to this latter class. Importantly, the presence of rare splice variants and artifactual ESTs was carefully evaluated. Subsequent cluster analysis of novel ESTs provided an indication of additional gene content in some cases. The resulting clusters were graphically displayed against the genomic sequence, providing indications of separate clusters that may contribute to the same gene, thereby facilitating development of confirmatory experiments in the laboratory. While much of this information was available in the public domain, the customized analysis performed revealed additional information not immediately apparent from the public genome browsers.

A unique consensus sequence was constructed for each splice variant and a trained reviewer assessed each alignment. This assessment included examination of all putative splice junctions for consensus splice donor/acceptor sequences, putative start codons, consensus Kozak sequences and upstream in-frame stops, and the location of polyadenylation signals. In addition, conserved noncoding sequences (CNSs) that could potentially be involved in regulatory functions were included as important information for each gene. The genomic reference and exon sequences were then archived for future reference. A master assembly that included all splice variants, exons and the genomic structure was used in subsequent analyses (i.e., analysis of polymorphisms). Table 3 lists gene clusters based on the publicly available EST and cDNA clustering algorithm, ECGene.

An important component of these efforts was the ability to visualize and store the results of the data mining efforts. A customized version of the highly versatile genome browser GBrowse (http://www.gmod.org/) was implemented in order to permit the visualization of several types of information against the corresponding genomic sequence. In addition, the results of the statistical analyses were plotted against the genomic interval, thereby greatly facilitating focused analysis of gene content.

Computational Analysis of Genes and GeneMap

In order to assist in the prioritization of candidate genes for which minimal annotation existed, a series of computational analyses were performed that included basic BLAST searches and alignments to identify related genes. In some cases this provided an indication of potential function. In addition, protein domains and motifs were identified that further assisted in the understanding of potential function, as well as predicted cellular localization.

A comprehensive review of the public literature was also performed in order to facilitate identification of information regarding the potential role of candidate genes in the pathophysiology of SCHIZOPHRENIA disease. In addition to the standard review of the literature, public resources (Medline and other online databases) were also mined for information regarding the involvement of candidate genes in specific signaling pathways. A variety of pathway and yeast two hybrid databases were mined for information regarding protein-protein interactions. These included BIND, MINT, DIP, Interdom, and Reactome, among others. By identifying homologues of genes in the SCHIZOPHRENIA candidate regions and exploring whether interacting proteins had been identified already, knowledge regarding the GeneMaps for SCHIZOPHRENIA disease was advanced. The pathway information gained from the use of these resources was also integrated with the literature review efforts, as described above.

Expression Studies

In order to determine the expression patterns for genes, relevant information was first extracted from public databases. The UniGene database, for example, contains information regarding the tissue source for ESTs and cDNAs contributing to individual clusters. This information was extracted and summarized to provide an indication in which tissues the gene was expressed. Particular emphasis was placed on annotating the tissue source for bona fide ESTs, since many ESTs mapped to Unigene clusters are artifactual. In addition, SAGE and microarray data, also curated at NCB, (Gene Expression Omnibus), provided information on expression profiles for individual genes. Particular emphasis was placed on identifying genes that were expressed in tissues known to be involved in the pathophysiology of schizophrenia (i.e. Brain-related tissues). To complement available information about the expression pattern of candidate disease genes, differents experimental approaches were used. The first one was a RT-PCR based semi-quantitative gene expression profiling method that could be applied to a large number of target sequences (genes, transcripts, ESTs) over a panel of 24 selected tissues. In some cases, where unexpected secondary PCR products were observed in Brain-related tissues, the PCR products were separated by agarose-gel electrophorese, purified and their DNA sequences was determined. The second approach was to map expression sites of mouse transcripts orthologous to a small set of human disease candidate genes in the mouse embryo (day 10.5, 12.5 and 15.5), in the postnatal stages (day 1 and 10) and at adulthood using in situ hybridization (ISH) method.

a. Semi-Quantitative Gene Expression Profiling by RT-PCR

Total human RNA samples from 24 different tissues Total RNA sample were purchased from commercial sources (Clontech, Stratagene) and used as templates for first-strand cDNA synthesis with the High-Capacity cDNA Archive kit (Applied Biosystems) according to the manufacturer's instructions. A standard PCR protocol was used to amplify genes of interest from the original sample (50 ng cDNA); three serial dilutions of the cDNA samples corresponding to 5, 0.5 and 0.05 ng of cDNA were also tested. PCR products were separated by electrophoresis on a 96-well agarose gel containing ethidium bromide followed by UV imaging. The serial dilutions of the cDNA provided semi-quantitative determination of relative mRNA abundance. Tissue expression profiles were analyzed using standard gel imaging software (AlphaImager 2200); mRNA abundance was interpreted according to the presence of a PCR product in one or more of the cDNA sample dilutions used for amplification. For example, a PCR product present in all the cDNA dilutions (i.e. from 50 to 0.05 ng cDNA) was designated ++++ while a PCR product only detectable in the original undiluted cDNA sample (i.e., 50 ng cDNA) was designated as + or +/−, for barely detectable PCR products (see Table 37). For each target gene, one or more gene-specific primer pairs were designed to span at least one intron when possible. Multiple primer-pairs targeting the same gene allowed comparison of the tissue expression profiles and controlled for cases of poor amplification.

The presence of secondary PCR products were observed in brain-related tissues for gene BCAS1 when amplified with primers spanning between exon 7 (213pb, primer Seq ID: 19489) and exon 10 (66pb, primer Seq ID: 19488) suggesting alternative splicing variants. The DNA sequence determination of 3 isoforms (see Table 38a, Seq IDs: 19619, 19620 and 19621) confirmed that the major isoform in Brain lacks the 168pb exon 9.

The validation by DNA sequencing of the brain's specific EST HS.573649, when amplified with primers located in the first and third putatives exons (Seq IDs: 19603 and 19604), respectively, also revealed alternative splicing variants (Table 38b, Seq IDs: 19622, 19623, 19624 and 19625) with a major isoform bearing an extra 54 bp exon inserted between exon 1 and 2 (Seq ID: 19623).

b. In Situ Hybridization (ISH) Study

### General Procedure:

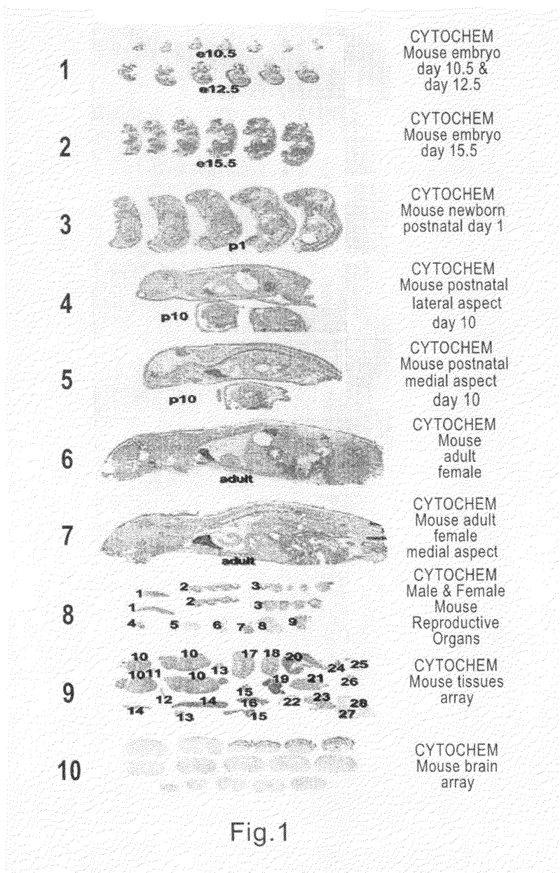

4 genes, highlighted in the GWAS study, namely Kmo, Cadm3, Ptprd and Tmeff2 were selected for further characterization by ISH in mouse. For each gene, a fragment of the mouse ortholog cDNA was use for the synthesis of cRNA probes (Table 36). To maximally preserve the integrity of tissue in its environment, mouse whole-body sections were used (FIG. 1). Whole bodies were frozen cut into 10-μm sections. To complement the whole-body sections, tissue arrays including reproductive organs (RO), general tissue array (TA) and brain array (BA) were used (FIG. 1). Tissue slices were mounted on glass microscope slides, fixed in formaldehyde and hybridized with ³⁵S-labeled cRNA probes. Antisense cRNA generated positive signals whereas sense cRNA (identical to mRNAs) generated negative (control) signals. Prior to gene-specific ISH, the tissues were validated with riboprobes to LDL receptor mRNA (data not shown). Following ISH, gene expression patterns were analyzed by both x-ray film autoradiography and emulsion autoradiography with appropriate exposure times.

### Detailed Procedure:

Mouse cDNA Clone and DNA Templates Preparation

cDNA clones of mouse orthologs to human genes Kmo, Cadm3, Ptprd and Tmeff2 were obtained from commercial source (Open Biosystem). DNA fragments to be used as templates for the cRNA probes synthesis were amplified by PCR and cloned into pGEM-7Zf(+)/LIC-F (ATCC #87048). After sequence validation, the templates for the antisense cRNA probes synthesis were generated by PCR using forward primers located at the 5′ end of the cloned DNA fragments and a reverse primer located upstream of the SP6 polymerase promoter (in the vector). Similarly, the templates for the sense (control) cRNA probes synthesis were generated by PCR using a forward primer located upstream of the T7 promoter (in the vector) and reverse primers located at the 3′ end of the cloned DNA fragments.

cRNA Probe Preparation

cRNA transcripts were synthesized in vitro from linear DNA fragments by run-off transcription with the SP6 or T7 RNA Polymerase from their respective promoters. Cold probe synthesis proved that DNA templates are functional and, hence, applied to radioactive probe synthesis labeled with ³⁵S-UTP (>1,000 Ci/mmol; Amersham).

### Tissues Preparation.

Tissues were frozen-cut into 10-μm sections, mounted on gelatin-coated slides and stored at −80° C. Before ISH, they were fixed in 4% formaldehyde (freshly made from paraformaldehyde) in phosphate-buffered saline (PBS), treated with triethanolamine/acetic anhydride, washed and dehydrated with a series of ethanol.

### Hybridization and Washing Procedures.

Sections were hybridized overnight at 55° C. in 50% deionized formamide, 0.3 M NaCl, 20 mM Tris-HCl, pH 7.4, 5 mM EDTA, 10 nM NaPO4, 10% dextran sulfate, 1×Denhardt's, 50 μg/ml total yeast RNA, and 50-80,000 cpm/μl ³⁵S-labeled cRNA probe. The tissue was subjected to stringent washing at 65° C. in 50% formamide, 2×SSC, and 10 mM DTT, followed by washing in PBS before treatment with 20 μg/ml RNAse A at 37° C. for 30 minutes. After washes in 2×SSC and 0.1×SSC for 10 minutes at 37° C., the slides were dehydrated, apposed to X-ray film for 5 days, then dipped in Kodak NTB nuclear track emulsion, and exposed for 12 days in light-tight boxes with desiccant at 4° C.

### Imaging.

Photographic development was undertaken with Kodak D-19. The slides were lightly counterstained with cresyl violet and analyzed under both light- and darkfield optics. Sense control cRNA probes (identical to mRNAs) always gave background levels of the hybridization signal.

### Storage and Rehydration

“Crystallization” of any section could be repaired by allowing the coverslips to fall off after soaking in xylene for 24-48 hours. The slides were rehydrated to 70% EtOH and then re-dehydrated again in a series of ethanol (80%, 96% and 2×100% for 2 minutes each). After 3 changes with xylene, the coverslips were mounted with Cytoseal (VWR Scientific) or other comparable mounting medium. Using the same method, the coverslips were removed for histological staining to take brightfield micrographs. Histological stains that require acidic conditions could dissolve silver grains. Overstaining could obscure the silver grains. Any excess mounting medium or residual emulsion on the back of the slides was removed with a single-edged razor. The re-coverslipped slides were dried flat for 24 hours, and stored indefinitely at room temperature.

### Viewing Original Slides

The results are best viewed by darkfield illumination, with ×2.5, ×4, ×10, ×25 and 40× objectives; the silver grains can be localized over particular cells. The antisense probe detects mRNA, and the sense control probe shows the background level of silver grains for the experiments.

### Results:

### Kmo

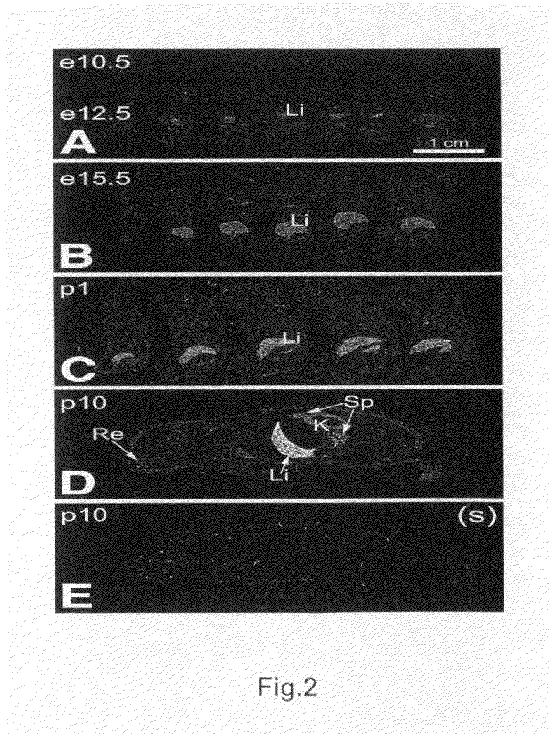

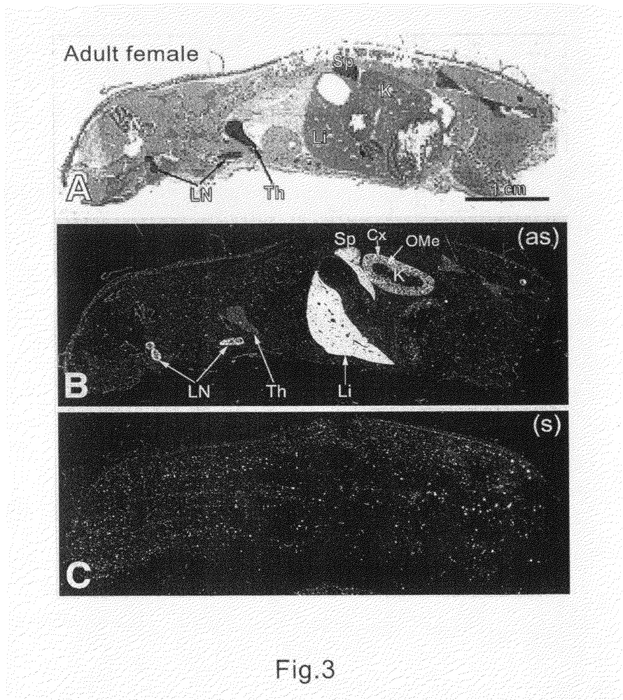

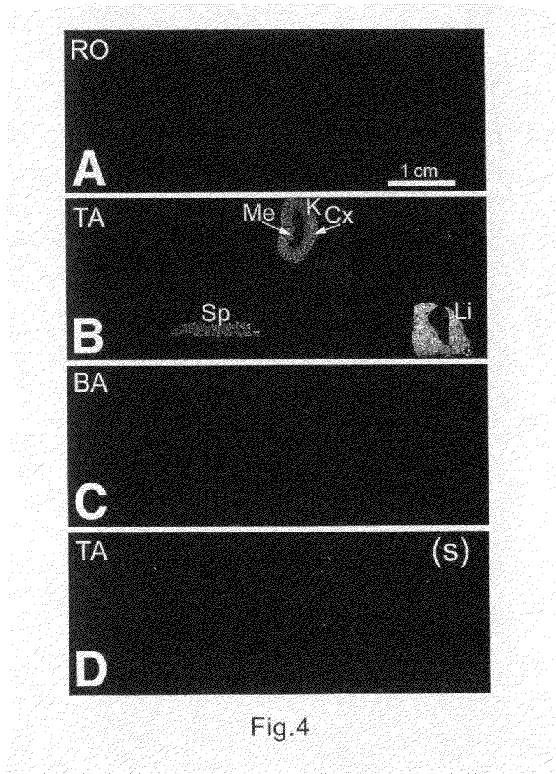

Following ISH, Kmo gene expression patterns were analyzed by both x-ray film autoradiography and emulsion autoradiography with exposure times of 4 days and 12 days, respectively. Results are presented in Table Z1 and FIGS. 2 to 8.

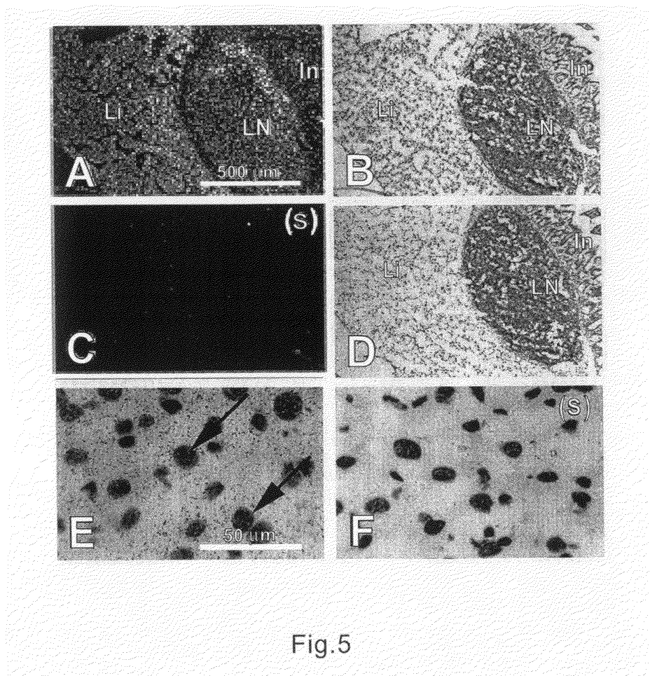

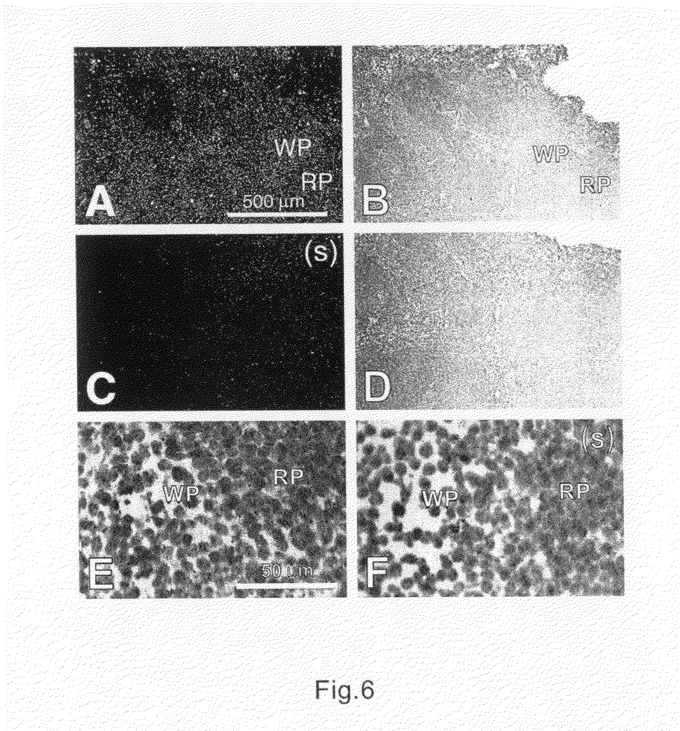

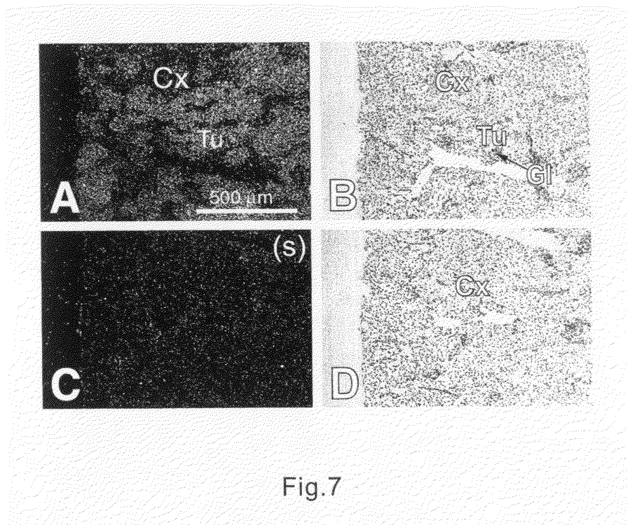

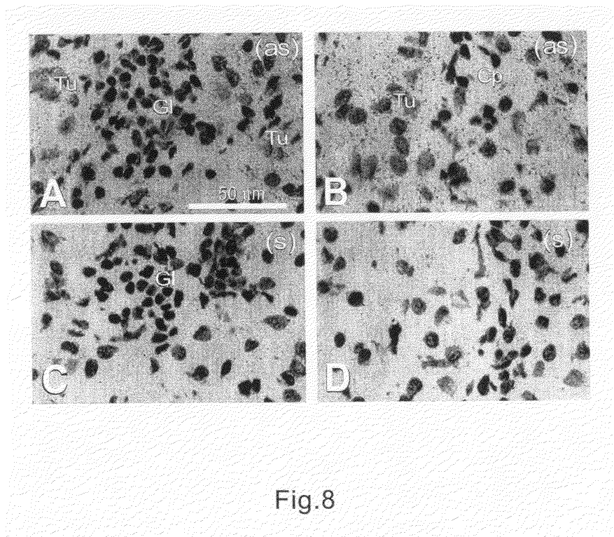

Analysis of ISH results provide evidence of Kmo expression in the specialized regions of the embryonic, newborn, postnatal and adult mice. Undetectable on embryonic day 10.5, ISH signal was evident on day 12.5 in the rudimental liver, persisting there along further developmental stages. The highest level of expression was noted to occur in the adult liver. The Kmo gene was clearly expressed in the hepatocytes (FIG. 5). Starting from birth to the adult stages, Kmo expression was also evident in the spleen and kidney tissue. In the spleen, low-level labelling was spread out over the organ, including the red pulp and white pulp regions (FIG. 6). In addition to the spleen, Kmo mRNA was also detected in the lymph nodes (FIG. 5), emphasizing its role in the body immunosurveillance process. In the kidney, Kmo expression was limited to the cortex and outer medulla, where the proximal and distal tubules, but not glomerulli, were labelled (FIGS. 7 and 8).

Kmo gene expression is characterized by high tissue specificity displaying a restricted pattern of mRNA distribution, with a presence in the liver, lymphatic tissue and kidney cortex. The highest level of expression was noted in the adult liver hepatocytes, suggesting its role in the hepatic metabolic/catabolic function.

TABLE Z1
Detection of KMO mRNA in whole body sections from 3 different
mouse ontogeny stages, 2 postnatal stages and adulthood

| | Development | | | |
|---|---|---|---|---|
| # | Day | Stage | SCORE | Comments |
| 1 | e10.5 | Embryo, midgestation | − | — |
| 2 | e12.5 | Embryo, midgestation | + | Very low-level expression in the liver |
| 3 | e15.5 | Embryo, lategestation | ++ | Low-level expression in the liver |
| 4 | P1 | Newborn | ++ | Low-level expression in the liver |
| 5 | P10 | Postnatal | +++ | Medium-level expression in the liver |
| | | | | (+++), spleen (+) and kidney (+) |
| 6 | P56-77 | Adulthood | ++++ | High-level expression in the liver |
| | | | | (++++), spleen (+++) and kidney (++). |

Average labeling level:
− = not detectable;
+ = very weak;
++ = weak;
+++ = medium; and
++++ = high and
+++++ = very high GENE13 mRNA concentration.

### Cadm3

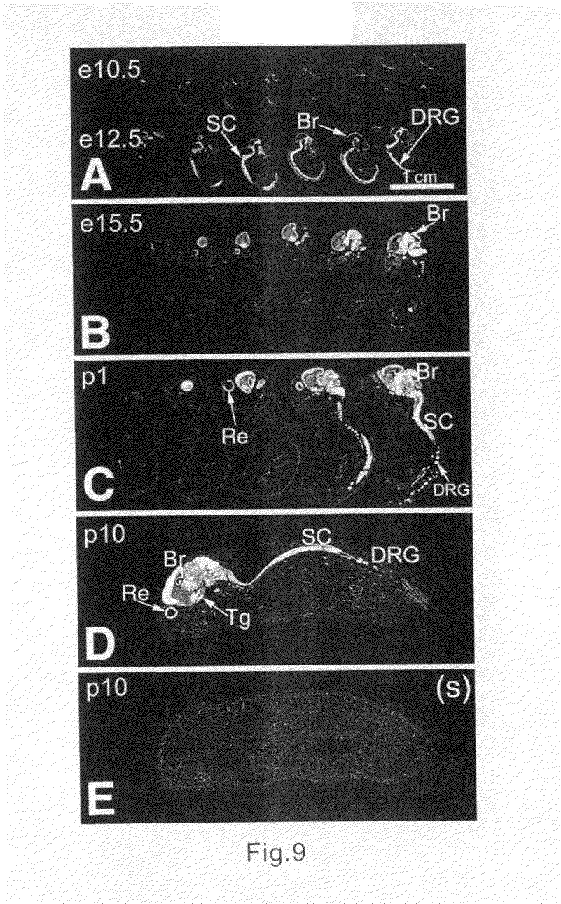

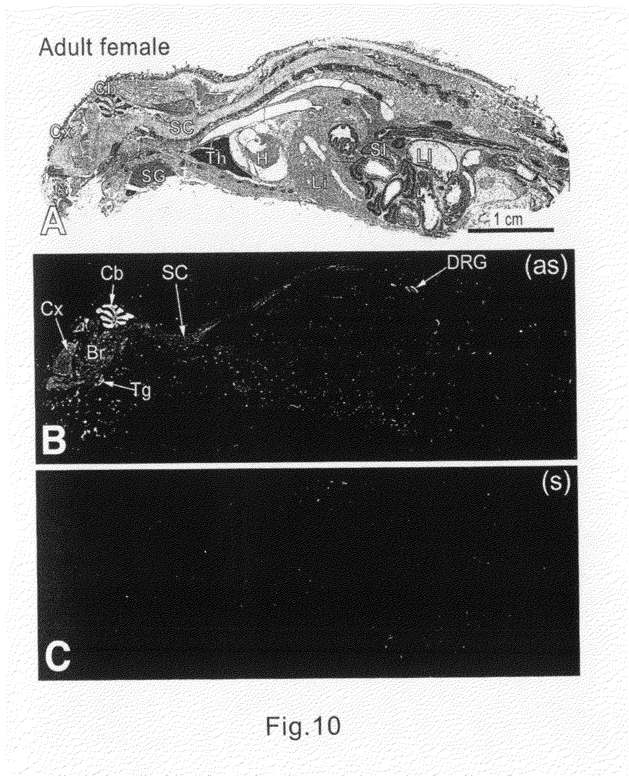

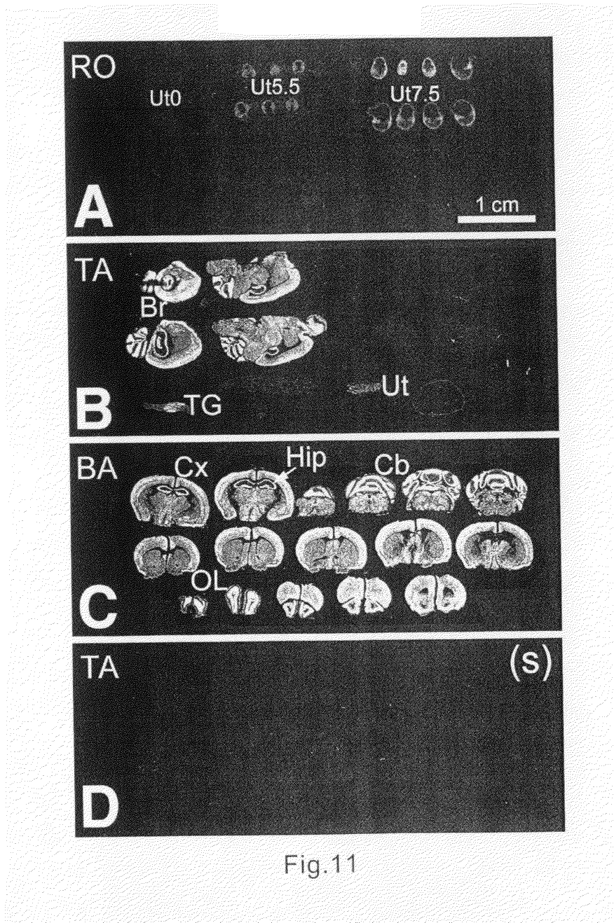

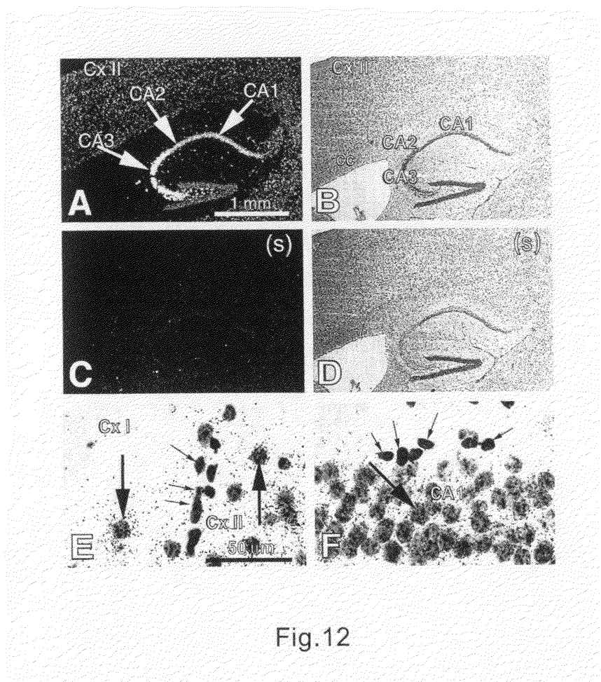

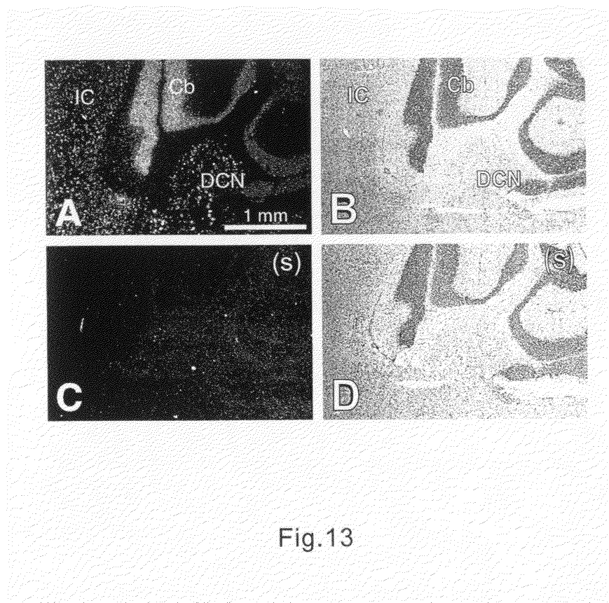

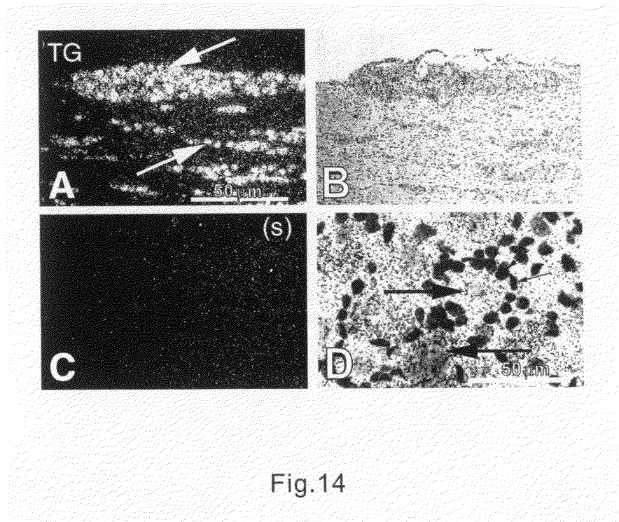

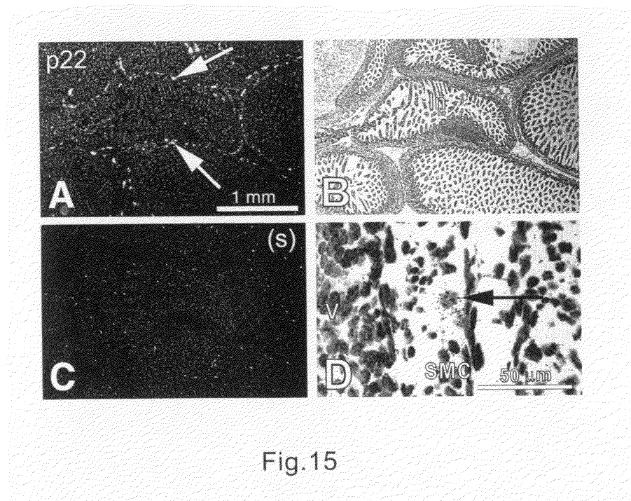

Following ISH, Cadm3 gene expression patterns were analyzed by both x-ray film autoradiography and emulsion autoradiography with exposure times of 3 days and 12 days, respectively. Results are presented in Tables Z2 and Z3 and FIGS. 9 to 15.

Analysis of ISH results provide evidence of Cadm3 expression in the central (CNS) and peripheral (PNS) nervous system of the embryonic, newborn, postnatal and adult mice. Light in e10.5 embryo, ISH signal increased significantly on day 12.5 and persisted elevated along further developmental stages. In the adult stage, when CNS architecture appears as fully developed, Cadm3 mRNA labelling was confined to grey matter clearly separated from unlabeled white matter. Labelled neurons displayed a widespread distribution in almost all CNS regions, showing Nissl-like pattern. Glial cells, ependymocytes, plexus choroids and endothelial cells in CNS appeared to be free of labelling. In the PNS, a presence of Cadm3 mRNA was noted in the cranial ganglia (trigeminal ganglion), dorsal root ganglia. During postnatal development, especially in p10 mice, there were labelled neurons in the intestinal wall, between smooth muscle fibres, forming a part of the plexus called plexus Auerbach. In the adult stage, Auerbach plexus appear to be free of labelling, suggesting by thus Cadm3 role of gut development rather than in adult intestine physiology.

Cadm3 gene expression is characterized by high tissue specificity displaying mRNA distribution pattern restricted to developing and adult CNS and PNS. The presence of Cadm3 mRNA specifically in the neuronal, but not glial cells suggests its neuronal function while its postnatal down-regulation in the plexus Auerbach suggests its role in the postnatal gut development.

TABLE Z2
Detection of CADM3 mRNA in whole body sections from 3
different mouse ontogeny stages, 2 postnatal stages and adulthood

| | Development | | | |
|---|---|---|---|---|
| # | Day | Stage | SCORE | Comments |
| 1 | e10.5 | Embryo, midgestation | + | Low-level expression in CNS and PNS |
| 2 | e12.5 | Embryo, midgestation | ++ | Medium-level expression in CNS and |
| | | | | PNS |
| 3 | e15.5 | Embryo, lategestation | ++++ | High-level expression in CNS and PNS |
| 4 | P1 | Newborn | +++++ | Very high-level expression in CNS and |
| | | | | PNS |
| 5 | P10 | Postnatal | +++++ | Very high-level expression in CNS and |
| | | | | PNS |
| 6 | P56-77 | Adulthood | +++++ | Very high-level expression in CNS and |
| | | | | PNS |

Average labelling level:
− = not detectable;
+ = very weak;
++ = weak;
+++ = medium; and
++++ = high and
+++++ = very high GENE15 mRNA concentration.

TABLE Z3
CADM3 mRNA tissue distribution in the adult mouse

| STRUCTURE | SCORE | COMMENTS |
|---|---|---|
| Section 1.01 Central nervous | +++++ | |
| system: | | |
| WHITE MATTER | − | |
| GREY MATTER | +++++ | |
| Cerebral cortex: | ++++ | |
| Neurons | ++++ | |
| Neuroblasts | ci | |
| Glial cells | − | |
| Circumventricular organs: | − | |
| Ependymocytes | − | |
| Tanycytes | − | |
| Choroid plexus | − | |
| Striatum: | ++ | |
| Hippocampus: | ++++ | |
| Hypothalamus: | ++ | |
| Thalamus: | ++ | |
| Epithalamus: | ++ | |
| Cerebellum: | +++ | |
| Medulla oblongata: | +++++ | |
| Spinal cord | +++ | |
| Section 1.02 Peripheral nervous | +++++ | |
| system: | | |
| Cranial ganglia: | +++++ | |
| Spinal ganglia: | +++++ | |
| Neurons | − | |
| Satelite cells | ne | |
| Paravertebral ganglia | ne | |
| Previsceral ganglia | − | ++ in p10 |
| Visceral plexus | − | |
| Peripheral nerves: | ++ | |
| Olfactory euroepithelium: | − | + in p1 |
| Retina | − | |
| Lens | ne | − in p1 |
| Corti organ | − | |
| Section 1.03 Circulatory system: | | |
| Section 1.04 Heart | | |
| Section 1.05 Blood Vessels | | |
| Respiratory System: | | |
| Nasal passage | | |
| Nasal mucosa | | |
| Trachea | | |
| Lung | − | |
| Section 1.06 Gastrointestinal | | |
| system: | | |
| Tongue | | |
| Oesophagus | | |
| Stomach | | |
| Small intestine | | |
| Large intestine | − | |
| Section 1.07 Gut associated | | |
| tissues: | | |
| Salivary gland | | |
| Exocrine pancreas | | |
| Liver | | |
| Gallbladder | − | |
| Section 1.08 Lymphatic tissues: | | |
| Thymus | | |
| Spleen | | |
| Lymphatic nodes | − | |
| Section 1.09 Endocrine System: | | |
| Pituitary gland | | |
| Thyroid | | |
| Parathyroid | | |
| Endocrine pancreas | − | |
| Adrenals | | |
| Section 1.10 Exocrine System: | | |
| Olfactory Bowman's glands | | |
| Lacrimal gland | | |
| Hardenia gland | | |
| Mammillary glands | | |
| Subaceus glands | − | |
| Sweet glands | | |
| Section 1.11 Urinary system: | | |
| Kidney | | |
| Cortex | | |
| Medulla | | |
| Urinary bladder | | |
| Section 1.12 Reproductive system: | ± | + in pregnant mouse |
| Ovary | | |
| Uterus | | |
| Testis | | |
| Epididymis | | |
| Seminal vesicle | | |
| Prostate | − | |
| Urethra | | |
| Skin: | | |
| Derma | | |
| Epidermis | − | |
| Hypodermis | | |
| Bone, Cartilage and Tooth: | | |
| Bone | | |
| Bone marrow | | |
| Cartilage: | | |
| Tooth | | |

Scale:
− = not detectable;
+ = weak;
++ = intermediate;
+++ = medium;
++++ = strong and
+++++ = very strong labelling;
ci = criteria insufficient to identify cell type at present condition.*;
ne = not examined.
*As the cell types were solely established based on their topography and morphology they are considered as presumptive only. Specific phenotype markers are required to identify cell type unambiguously.

### Ptprd

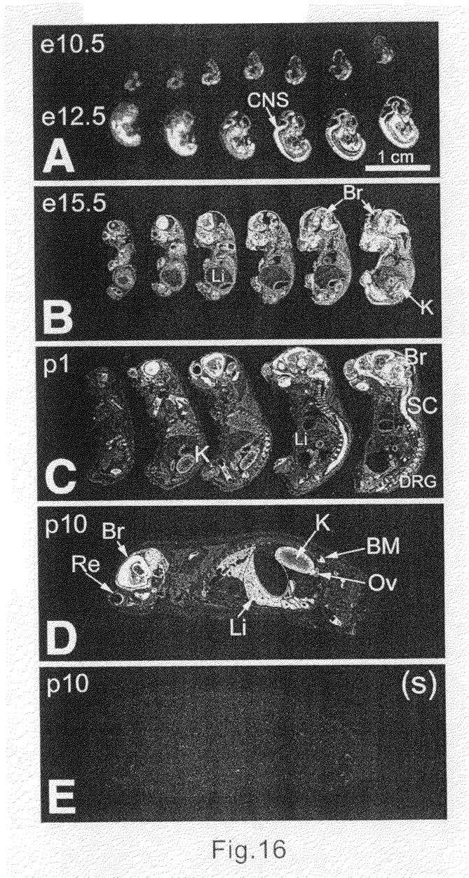

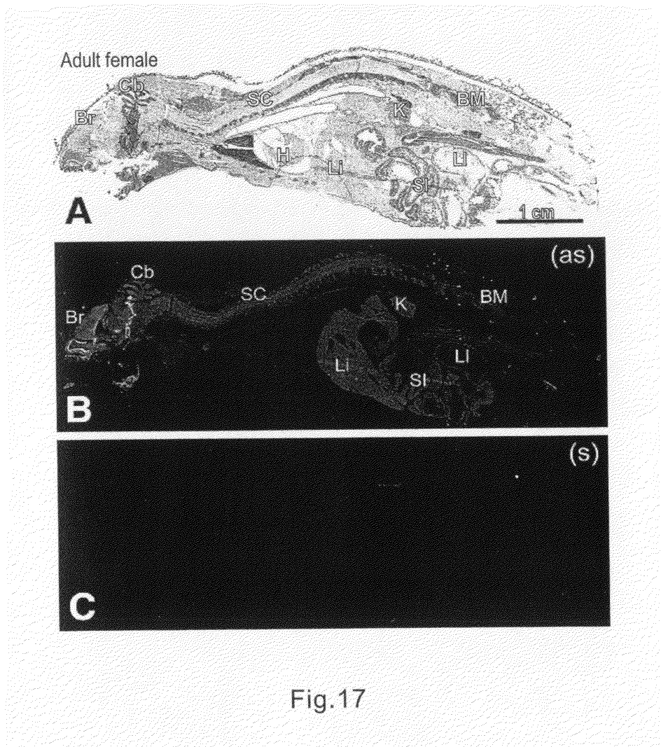

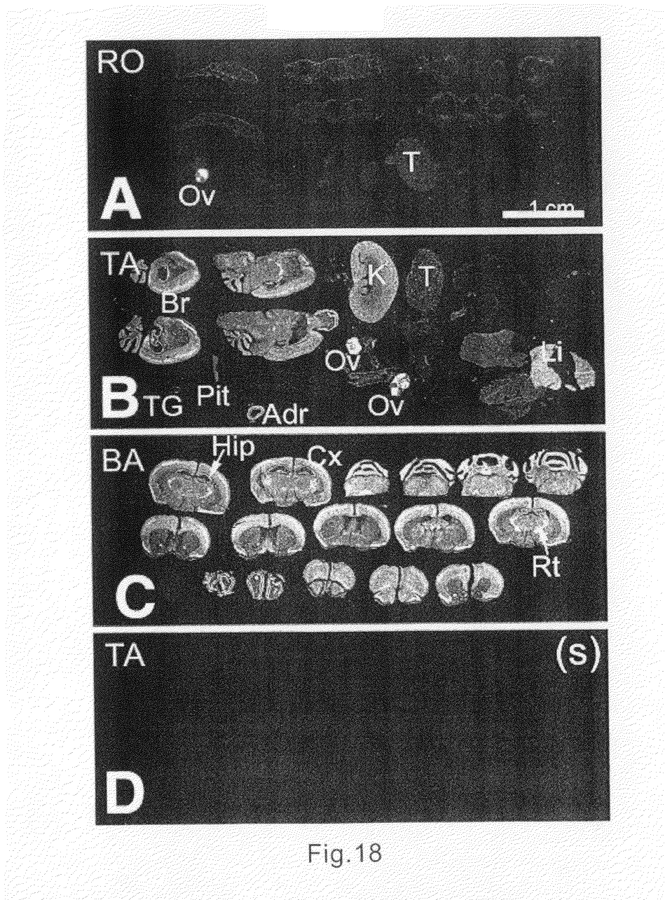

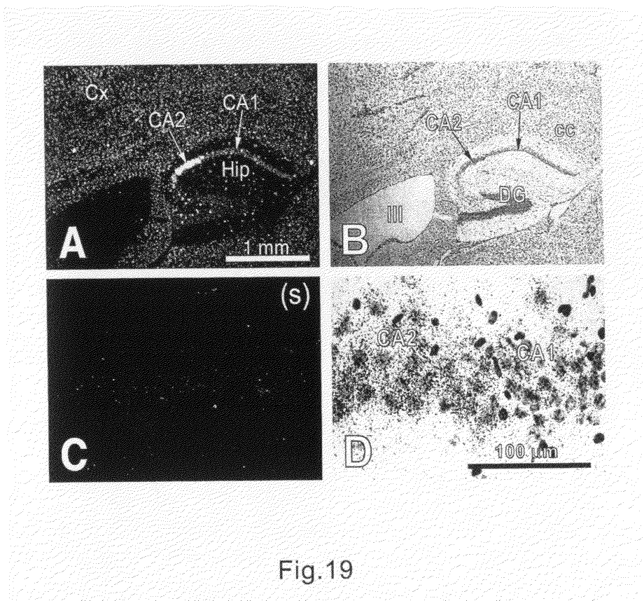

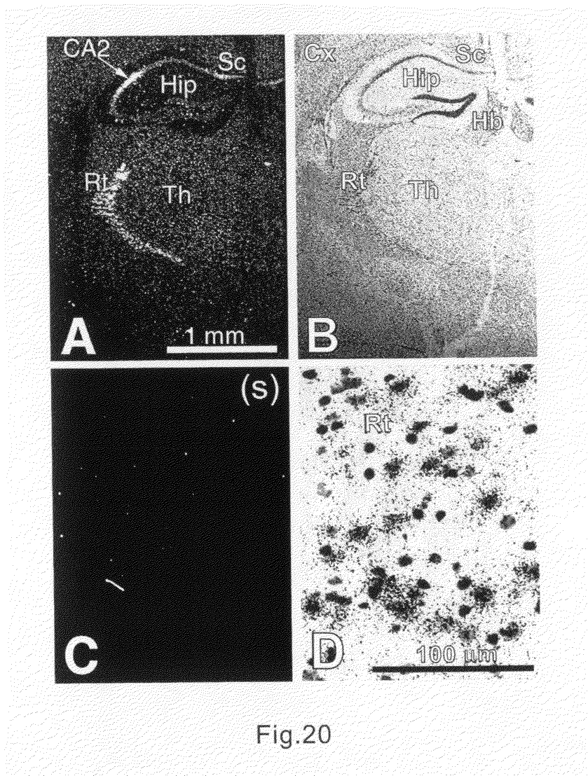

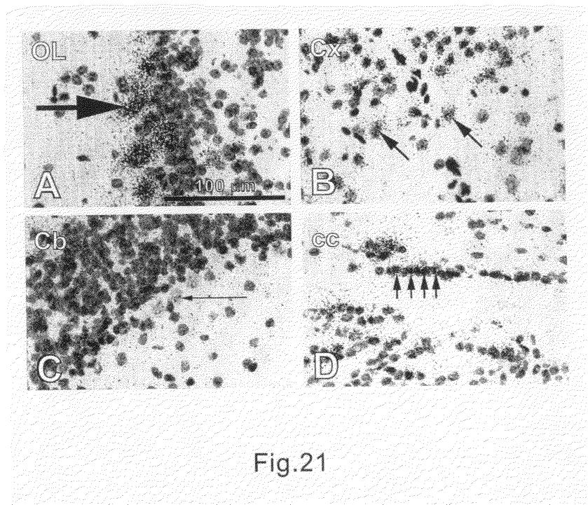

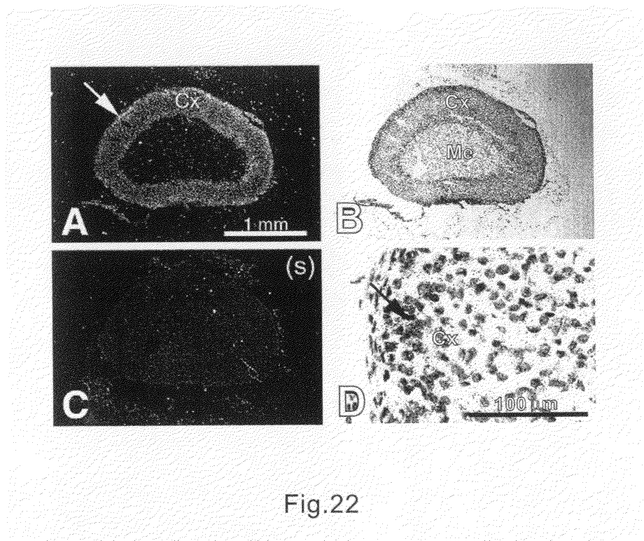

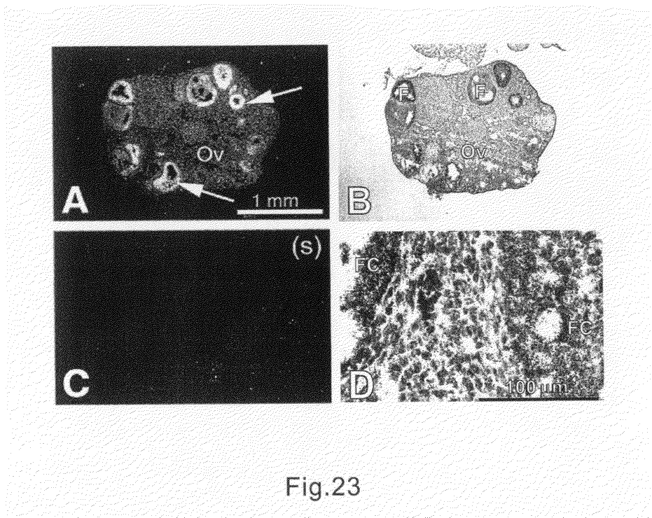

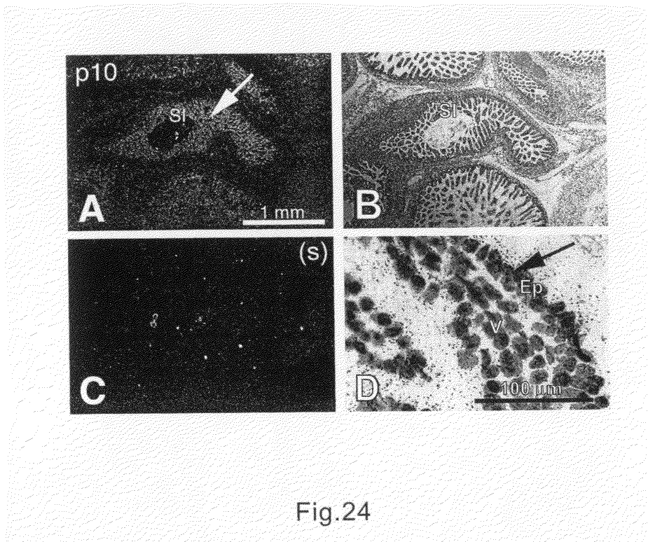

Following ISH, Ptprd gene expression patterns were analyzed by both x-ray film autoradiography and emulsion autoradiography with exposure times of 2 days and 10 days, respectively. Results are presented in Table Z4 and Z5 and FIGS. 16 to 24.

Analysis of ISH results provide evidence of Ptprd expression in the embryonic, newborn, postnatal and adult mice multiple regions including the central nervous system (CNS) and peripheral tissues. The onset time of Ptprd expression in different tissues is indicated in Table Z4. Light in e10.5 embryo, ISH signal increases significantly on day 12.5 and persists elevated along further developmental stages. Early expression was noted in e10.5 CNS, whereas late expression was observed in other regions: e12.5—gut; e15.5—kidney and lung; p1—adrenal gland and bone marrow, and p10—liver.

In the adult CNS Ptprd mRNA labelling formed a heterogeneous distribution pattern. Most labelling was found to be in a subpopulation of neuronal cells in the grey matter. If compare the large size neurons in the CNS, labelling intensity varied from one region to another, being high in the olfactory lobe mitral cells, moderate in the hippocampus pyramidal neurons, low in the cortex pyramidal cells and null in Purkinje cells of the cerebellum. This comparison indicates a regional specialization of Ptprd function. Among many labelled regions some are of interest to mental health. These are the hippocampal area 2 (CA2) involved in the stress regulation and the reticular thalamic nucleus (Rt), part of the brain visual tract, which is systemic to hallucinations in schizophrenia. In the white matter, Ptprd moderate labelling occurred in a subpopulation of the oligodendrocyte-like cells, which are known to produce myelin sheaths around the bundles of axon in CNS, indicating that Ptprd plays a role in the myelin production.

In addition to the nervous tissue, Ptprd mRNA was detected in the adrenal gland cortex. Higher concentration Ptprd mRNA was noted in a foremost peripheral zone known to contain aldosterone producing cells. Other endocrine cells containing tissues studied such as the pituitary gland, thyroid, gut and pancreas were not labelled. As summarized in the Table Z5, Ptprd mRNA was observed in the adult mouse hepatocytes in the liver, follicular cells in the ovary.

In conclusion, Ptprd gene expression is characterized by a widespread heterogeneous pattern of distribution throughout the multiple tissues observed along mouse ontogeny (Table Z4). In the central nervous system, Ptprd expression starts at midgestation and lasts until adulthood. During CNS ontogeny, Ptprd mRNA distribution pattern changes from homogeneous to heterogeneous, long-lasting within specific centres highly labelled. Some of these centers are involved in stress control (hippocampal area CA2 and specific hypothalamic regions), and visual tract reticular thalamic nucleus, involved in the hallucination in shizophrenia, suggesting that Ptprd might have a role to play in these conditions. Furthermore, the presence of Ptprd mRNA in the nervous system is not limited to neuronal cells, since, the labelled oligodendrocyte that produce myelin sheaths around the bundles of axons were observed in the white matter regions, such as corpus callosum in the brain. Ptprd may, thus, be involved in the myelin production in the white matter. Finally, most tissues including CNS, gut, kidney, adrenal gland, bone marrow and liver display a long-lasting pattern of Ptprd expression, each having its own onset time of expression, whether prenatal (most tissues) or postnatal (liver). Interestingly, the lung tissue displays a transient, two-peak pattern of expression (see Table Z4 and FIG. 16), suggesting a biphasic gene regulation mechanism including (i) an up-regulation event and (ii) a repression step. Altogether, the tissue specificity and the stage-wise gene expression characteristics suggest that combination of the followings may account to Ptprd function: (1) several Ptprd mRNA isoforms exist; (2) multiple, tissue-specific promoters regulate a gene expression; (3) differential splicing occurs in tissue-specific manner and (4) target gene expression repression mechanism operates. PtPRd-derived products might, thus, represent a target for both developmental and non-developmental gene expression regulatory factors, including a stress pathway in CNS.

TABLE Z4
Detection of PTPRD mRNA in mouse ontogeny

| # | Devel. Day | Stage | CNS | Gut | Kidney | Lung | Adr. | Bone | Liver |
|---|---|---|---|---|---|---|---|---|---|
| 1 | e10.5 | Midgestation | + | − | − | − | − | − | − |
| 2 | e12.5 | Midgestation | ++ | +++ | − | − | − | − | − |
| 3 | e15.5 | Lategestation | ++++ | +++ | ++ | +++ | + | ++ | − |
| 4 | P1 | Newborn | +++++ | ++ | +++ | + | +++ | +++ | − |
| 5 | P10 | Postnatal | +++++ | ++ | +++ | +++ | ne | +++ | +++ |
| 6 | P56-77 | Adulthood | +++ | + | ++ | − | +++ | ++ | +++ |

Average labelling level:
− = not detectable;
+ = very weak;
++ = weak;
+++ = medium; and
++++ = high and
+++++ = very high GENE17 mRNA concentration;
ne—not examined.

TABLE Z5
PTPRD mRNA tissue distribution in the adult mouse

| STRUCTURE | SCORE | COMMENTS |
|---|---|---|
| Section 1.13 Central nervous | | |
| system: | | |
| WHITE MATTER | ++ | |
| GREY MATTER | ++++ | |
| Cerebral cortex: | ++ | |
| Neurons | ++ | |
| Neuroblasts | − | |
| Glial cells | − | |
| Circumventricular organs: | − | |
| Ependymocytes | − | |
| Tanycytes | − | |
| Choroid plexus | − | |
| Striatum: | + | |
| Hippocampus: | +++ | |
| Hypothalamus: | ++ | |
| Thalamus: | ++++ | |
| Epithalamus: | + | |
| Cerebellum: | + | |
| Medulla oblongata: | +++ | |
| Spinal cord | +++ | |
| Section 1.14 Peripheral nervous | + | |
| system: | | |
| Cranial ganglia: | + | |
| Spinal ganglia: | + | |
| Neurons | − | |
| Satelite cells | − | |
| Paravertebral ganglia | − | |
| Previsceral ganglia | − | |
| Enteric plexus | − | |
| Peripheral nerves: | + | |
| Olfactory euroepithelium: | ne | +++ in p10 |
| Retina | ne | − in p10 |
| Lens | ne | + in p10 |
| Corti organ | | |
| Section 1.15 Circulatory system: | − | |
| Section 1.16 Heart | | |
| Section 1.17 Blood Vessels | | |
| Respiratory System: | | |
| Nasal passage | | |
| Nasal mucosa | | |
| Trachea | | |
| Lung | − | |
| Section 1.18 Gastrointestinal | ne | |
| system: | | |
| Tongue | − | |
| Oesophagus | + | |
| Stomach | + | |
| Small intestine | | |
| Large intestine | − | |
| Section 1.19 Gut associated | − | |
| tissues: | | |
| Salivary gland | ++ | |
| Exocrine pancreas | − | |
| Liver | − | |
| Gallbladder | | |
| Section 1.20 Lymphatic tissues: | | |
| Thymus | | |
| Spleen | | |
| Lymphatic nodes | | |
| Section 1.21 Endocrine System: | | |
| Pituitary gland | | |
| Thyroid | ++ | |
| Parathyroid | − | |
| Endocrine pancreas | | |
| Adrenals | | |
| Section 1.22 Exocrine System: | | |
| Olfactory Bowman's glands | | |
| Lacrimal gland | | |
| Hardenia gland | | |
| Mammillary glands | ++ | |
| Subaceus glands | ++ | |
| Sweet glands | − | |
| Section 1.23 Urinary system: | − | |
| Kidney | | |
| Cortex | +++ | |
| Medulla | − | |
| Urinary bladder | + | |
| Section 1.24 Reproductive system: | − | |
| Ovary | | |
| Uterus | − | |
| Testis | − | |
| Epididymis | ne | |
| Seminal vesicle | − | |
| Prostate | | |
| Urethra | | |
| Skin: | | |
| Derma | | |
| Epidermis | | |
| Hypodermis | | |
| Bone, Cartilage and Tooth: | | |
| Bone | | |
| Bone marrow | | |
| Cartilage: | | |
| Tooth | | |

Scale:
− = not detectable;
+ = weak;
++ = intermediate;
+++ = medium;
++++ = strong and
+++++ = very strong labelling;
ci = criteria insufficient to identify cell type at present condition.*;
ne = not examined.
*As the cell types were solely established based on their topography and morphology they are considered as presumptive only. Specific phenotype markers are required to identify cell type unambiguously.

### Tmeff2

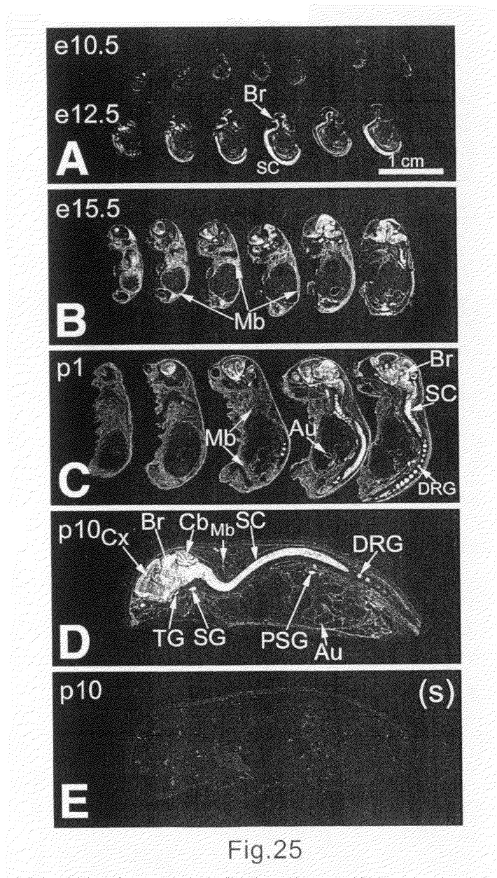

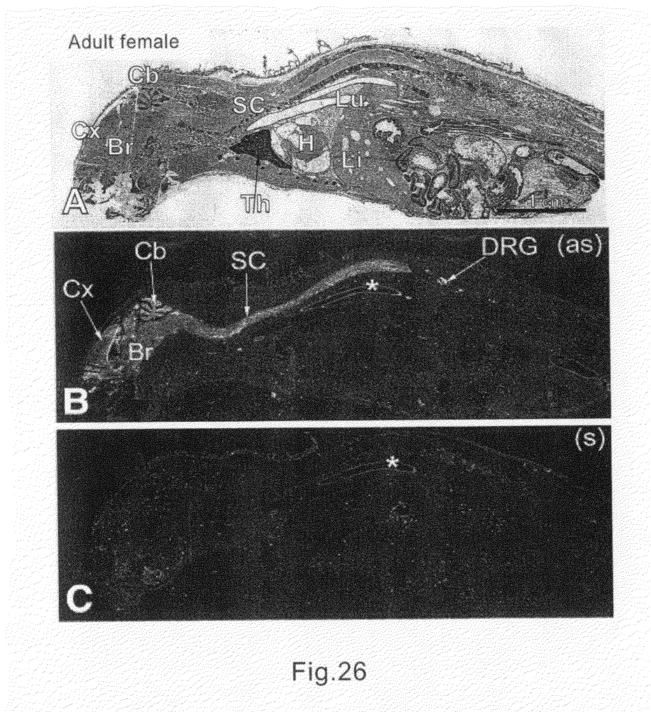

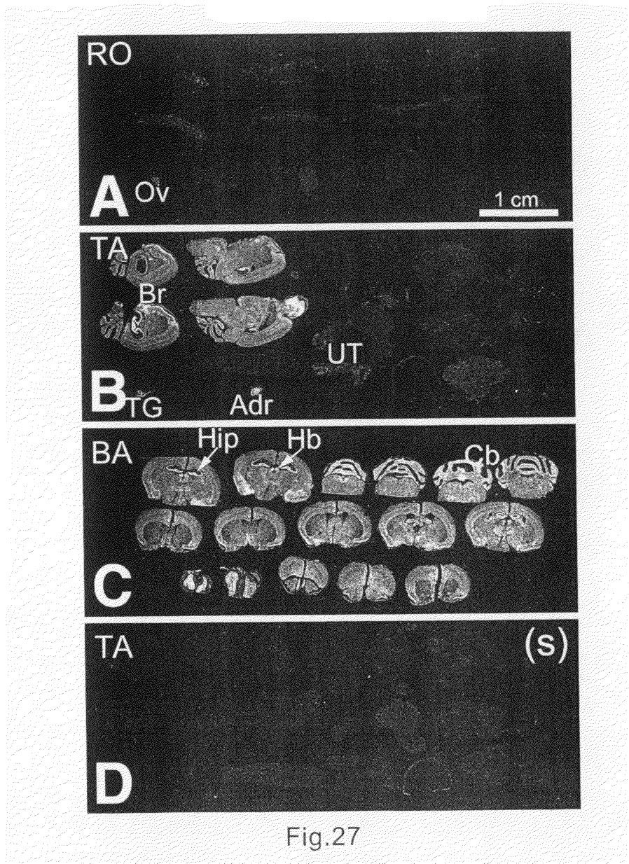

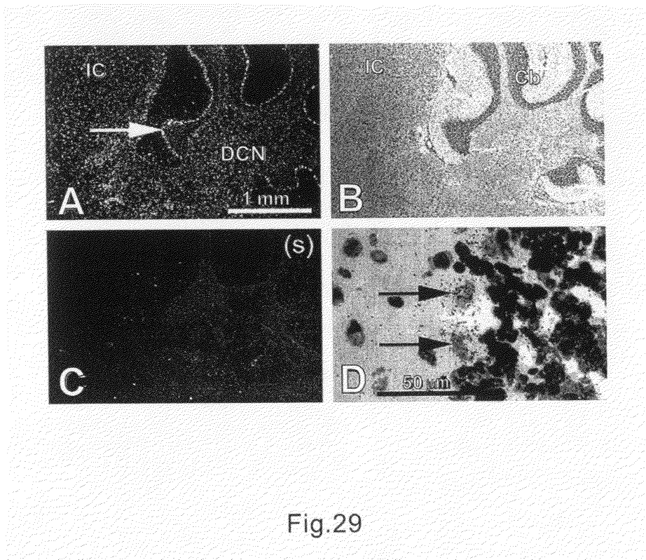

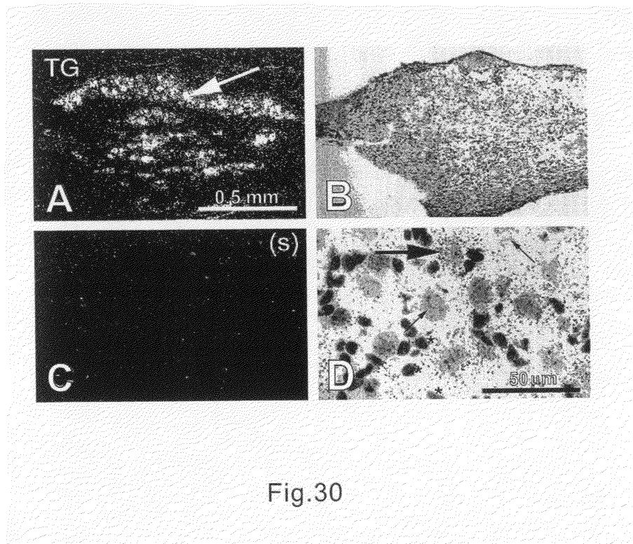

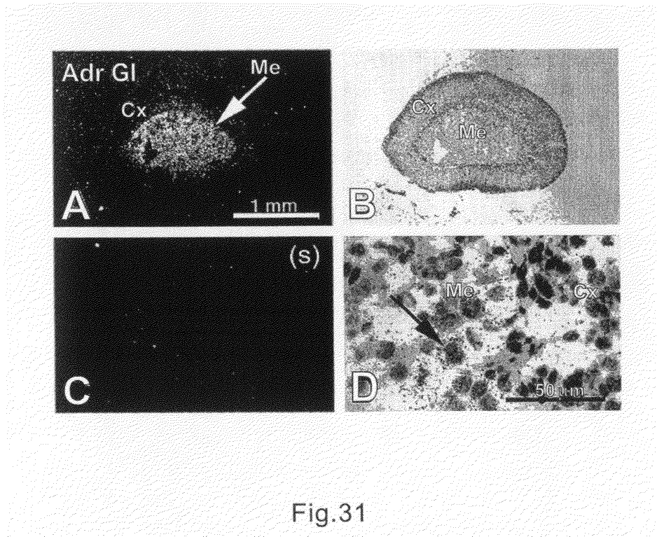

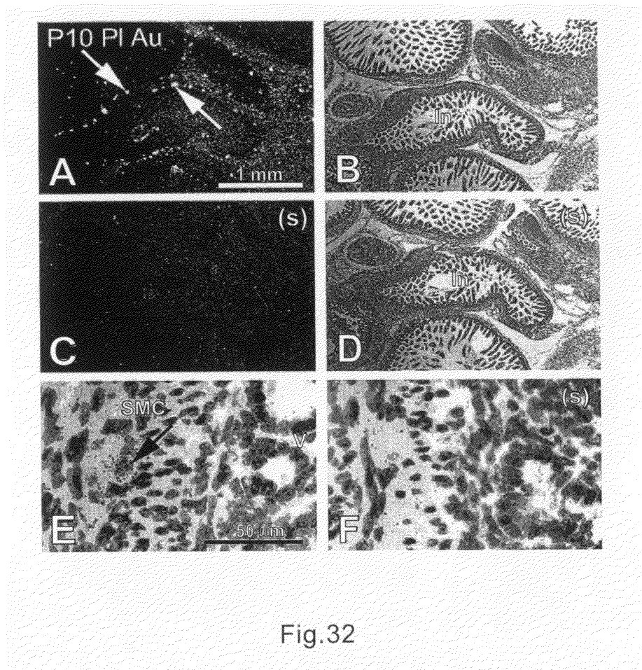

Following ISH, Tmeff2 gene expression patterns were analyzed by both x-ray film autoradiography and emulsion autoradiography with exposure times of 4 days and 16 days, respectively. Results are presented in Table Z6 and Z7 and FIGS. 25 to 32.

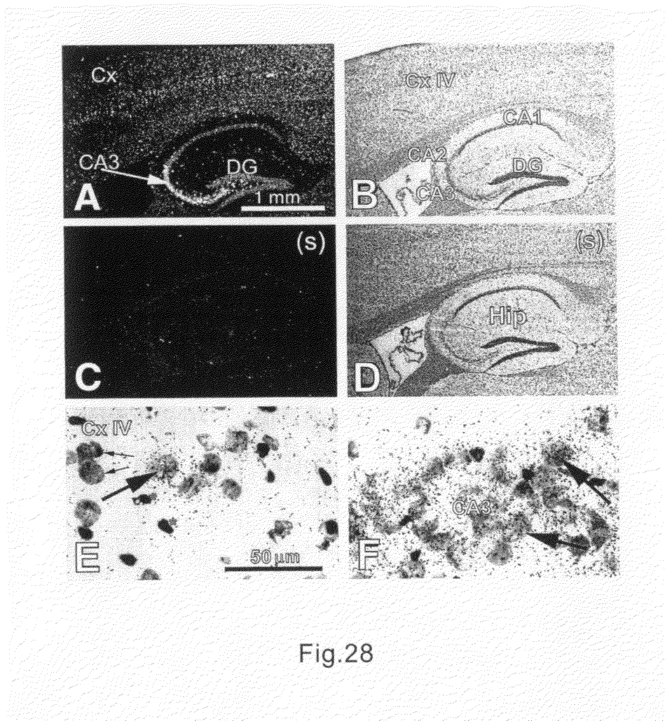

Analysis of ISH results provide evidence of Tmeff2 expression in the central (CNS) and peripheral (PNS) nervous system of the embryonic, newborn, postnatal and adult mice. Light in e10.5 embryo, ISH signal increases significantly on day 12.5 and persists elevated along further developmental stages. In the adult stage, when CNS architecture appears as fully developed with grey matter clearly delineated from white matter, Tmeff2 mRNA labelling appears to be confined to a former and absent in the letter. Glial cells, ependymocytes, plexus choroids and endothelial cells in CNS appeared to be free of labelling. Labelled neurons displayed a widespread distribution in almost all CNS regions, showing Nissl-like pattern. However, at closer examination performed under high microscopic magnification it appears that proportion of neurons, present for example in the cerebral cortex, remains unlabelled (FIG. 28E). For this reason, Tmeff2 expression pattern cannot be termed as pan-neuronal-like, but a widespread neuron-specific expression pattern.

In the PNS, a presence of Tmeff2 mRNA was noted in the neurons, but not in supportive satellite cells of the cranial ganglia such as trigeminal ganglion, spinal ganglia such as dorsal root ganglia, paravertebral sympathetic ganglia and gastrointestinal plexus. The later was especially evident during prenatal and postnatal development. Labelled enteric neurons present in the space in the intestinal wall, in between the two smooth muscle layers, inner circular and outer longitudinal, take part of the enteric plexus called Auerbach's plexus. In the adult stage, Auerbach plexus appear to be much less labelled, suggesting by thus Tmeff2 role mainly in the gut development. A role of Tmeff2 in the gastrointestinal nerve supply could potentially be a control of the peristalsis.

In addition to the nervous tissue, Tmeff2 mRNA was detected in the adrenal gland and the supportive tissue. Presence of Tmeff2 mRNA in the adrenal gland was limited to the medulla containing adrenergic/peptidergic cells, whereas the cortex where corticoids are synthesized remained unlabelled. Other endocrine cells containing tissues studied such as the pituitary gland, thyroid, gut and pancreas were not labelled.

Supportive tissues, especially the fibroblasts in the membranes around skeletal muscles and certain bones (i.e. cranial bones and phalanges) displayed Tmeff2 mRNA labelling. The level of Tmeff2 expression seems to be maximal in late prenatal development, was pronounced in the postnatal stage and low in the adult mice.

In conclusion, Tmeff2 gene expression displays a high-degree of tissue specificity, characterized by mRNA distribution restricted to the CNS, PNS, adrenal medulla and membranes. Expression of Tmeff2 in the supportive membranes around the muscles and skeleton suggests an interaction between the membrane fibroblasts and target cells in their growth and maintain. Otherwise said, Tmeff2 could be responsible for any malformation in the musculature and skeleton if cell-to-cell interaction depended upon its function. The presence of Tmeff2 mRNA in the nervous system, specifically in the neuronal, but not glial cells, suggests its neuronal function in a large number of regions. Finally, the expression of Tmeff2 in the enteric Auerbach's plexus suggests its role in the gut growth, probably influencing the set up of musculature and a subsequent peristalsis. Whether other body smooth musculature that control the iris, blood vessels and skin hairs receives Tmeff2 nerve supply is presently not known and merits further investigation in view to test Tmeff2 as CNS and PNS patho-physiology marker. As it is known, muscular tissue constitutes an excellent support to studies in genetics and pharmacology. Muscular tissue is also an excellent target to elaborate and test the diagnostic/prognostic tools to gene-encoded disease of the nervous system, whenever central or peripheral, or both.

TABLE Z6
Detection of TMEFF2 mRNA in whole body sections from 3
different mouse ontogeny stages, 2 postnatal stages and adulthood

| | Development | | | |
|---|---|---|---|---|
| # | Day | Stage | Score | Comments |
| 1 | e10.5 | Embryo, midgestation | + | Low-level expression in CNS and PNS |
| 2 | e12.5 | Embryo, midgestation | ++ | High-level expression in CNS and PNS; |
| | | | | Medium-level in the membranes |
| 3 | e15.5 | Embryo, lategestation | ++++ | High-level expression in CNS, PNS |
| | | | | and membranes |
| 4 | P1 | Newborn | +++++ | Very high-level in CNS and PNS; |
| | | | | Medium-level in the membranes |
| 5 | P10 | Postnatal | +++++ | Very high-level in CNS and PNS; |
| | | | | Low-level expression in the membranes |
| 6 | P56-77 | Adulthood | ++++ | High-level in CNS and PNS; |
| | | | | Low-level expression in the membranes |

Average labelling level:
− = not detectable;
+ = very weak;
++ = weak;
+++ = medium; and
++++ = high and
+++++ = very high GENE19 mRNA concentration.

TABLE Z7
TMEFF2 mRNA tissue distribution in the adult mouse

| STRUCTURE | SCORE | COMMENTS |
|---|---|---|
| Section 1.25 Central nervous | | |
| system: | | |
| WHITE MATTER | − | |
| GREY MATTER | +++ | |
| Cerebral cortex: | +++ | |
| Neurons | +++ | |
| Neuroblasts | − | |
| Glial cells | − | |
| Circumventricular organs: | − | |
| Ependymocytes | − | |
| Tanycytes | − | |
| Choroid plexus | − | |
| Striatum: | ± | |
| Hippocampus: | +++ | |
| Hypothalamus: | ++ | |
| Thalamus: | ++++ | |
| Epithalamus: | ++++ | |
| Cerebellum: | ++++ | |
| Medulla oblongata: | ++++ | |
| Spinal cord | ++ | |
| Section 1.26 Peripheral nervous | ++++ | |
| system: | | |
| Cranial ganglia: | ++++ | |
| Spinal ganglia: | ++++ | |
| Neurons | − | |
| Satelite cells | ne | +++++ in p1 |
| Paravertebral ganglia | ne | +++ in p10 |
| Previsceral ganglia | ± | ++++ in e15.5 |
| Enteric plexus | − | |
| Peripheral nerves: | ne | +++ in p10 |
| Olfactory euroepithelium: | ± | |
| Retina | − | |
| Lens | − | |
| Corti organ | − | |
| Section 1.27 Circulatory system: | | |
| Section 1.28 Heart | | |
| Section 1.29 Blood Vessels | | |
| Respiratory System: | | |
| Nasal passage | | |
| Nasal mucosa | | |
| Trachea | | |
| Lung | − | |
| Section 1.30 Gastrointestinal | | |
| system: | | |
| Tongue | | |
| Oesophagus | | |
| Stomach | | |
| Small intestine | | |
| Large intestine | − | |
| Section 1.31 Gut associated | | |
| tissues: | | |
| Salivary gland | | |
| Exocrine pancreas | | |
| Liver | − | |
| Gallbladder | | |
| Section 1.32 Lymphatic tissues: | | |
| Thymus | | |
| Spleen | | |
| Lymphatic nodes | − | |
| Section 1.33 Endocrine System: | − | |
| Pituitary gland | ne | |
| Thyroid | − | |
| Parathyroid | +++ | |
| Endocrine pancreas | − | |
| Adrenals | | |
| Section 1.34 Exocrine System: | | |
| Olfactory Bowman's glands | | |
| Lacrimal gland | | |
| Hardenia gland | | |
| Mammillary glands | | |
| Subaceus glands | − | |
| Sweet glands | | |
| Section 1.35 Urinary system: | | |
| Kidney | | |
| Cortex | | |
| Medulla | − | |
| Urinary bladder | | |
| Section 1.36 Reproductive system: | | |
| Ovary | | |
| Uterus | | |
| Testis | | |
| Epididymis | | |
| Seminal vesicle | − | |
| Prostate | | |
| Urethra | | |
| Skin: | | |
| Derma | − | |
| Epidermis | | |
| Hypodermis | | |
| Bone, Cartilage and Tooth: | | |
| Bone | | |
| Bone marrow | | |
| Cartilage: | | |
| Tooth | | |

Scale:
− = not detectable;
+ = weak;
++ = intermediate;
+++ = medium;
++++ = strong and
+++++ = very strong labelling;
ci = criteria insufficient to identify cell type at present condition.*;
ne = not examined.
*As the cell types were solely established based on their topography and morphology they are considered as presumptive only. Specific phenotype markers are required to identify cell type unambiguously.

### Schizophrenia Genemap and Pathways

The GWAS, and subsequent data mining analyses resulted in a compelling GeneMap that contains networks and pathways highly relevant to schizophrenia. The emerging GeneMap includes both novel and known pathways in neurological development, synaptic plasticity, learning, memory and other neurological disorders. Other identified regions contain genes with biological function relevant for the central nervous system or associated with neurological conditions such as spastic paraplegia.

### Link to Schizophrenia Pathway:

This pathway includes genes that have been already reported to be associated with schizophrenia, such as KCNN3, KMO, VDR, and NRG1. Other genes such as DISC1 and DTNBP1, have been repeatedly reported to be linked with the disease and connect directly to genes from our findings.

A signal pointing at the 5′ end of the Neuregulin 1 gene was found among the regions in paranoid sub-phenotype analysis. The NRG1 gene is expressed at synapses in the central nervous system and has an important role in the expression and activation of neurotransmitter receptors. The association of NRG1 with schizophrenia has been replicated in various populations. NRG1 codes for many mRNA species and different proteins via alternative splicing; it is thought to code for about 15 proteins with a diverse range of functions in the brain, including axon guidance, synaptogenesis, neurotransmission, etc. Any of these forms could potentially influence susceptibility to schizophrenia.

The KCNN3 gene encodes a potassium channel and it is epistatic to PTPRD, the top signal from the full sample analysis. KCNN3 is ubiquitously expressed across a variety of tissues. The first exon contains a polymorphic CAG repeats translating in a polyglutamine repeat in the protein. Several reports have shown evidence for a possible association of CAG expansion at this locus with schizophrenia and it has been suggested that variations in the length of the polyglutamine repeats produces subtle alterations in channel function, thus altering neuronal behavior.

Vitamin D3 receptor (VDR), is an intracellular hormone receptor that specifically binds the active form of vitamin D (1,25-dihydroxyvitamin D3). Our data show that this gene is in heterogeneity with PAFAH1B1 (LIS1), a gene identified in the full sample analysis. In animal models, the expression of VDR in the embryonic rat brain has been shown to rise steadily between embryonic days 15 and 23. Also, vitamin D has been shown to induce the expression of nerve growth factor and to stimulate neurite outgrowth in embryonic hippocampal explant cultures. In the neonatal rats low prenatal vitamin D in utero has been shown to lead to brain anomalies. Exposure to low levels of vitamin D during early human life is known to alter brain development and it is considered as a risk factor for schizophrenia. The KMO gene is located in the chromosome region 1q42-q44, a region associated with schizophrenia by linkage analysis. Polymorphisms in this gene have been shown to be associated with schizophrenia. Kynurenine 3-mono-oxygenase (KMO) inhibitors increase brain kynurenic acid (KYNA) synthesis and cause pharmacological actions possibly mediated by a reduced activity of excitatory synapses. Metabolic variations in the KYNA pathway have been suggested to be related to the etiology of schizophrenia. Finally, in situ hybridization experiment in mouse during different stage of development revealed that KMO is characterized by high tissue specificity displaying a restricted pattern of mRNA distribution, with a presence in the liver, lymphatic tissue and kidney cortex. The highest level of expression was noted in the adult liver hepatocytes, suggesting its role in the hepatic metabolic/catabolic function.

### Neurological Disorder Pathway:

This pathway includes genes such as APP, TAU, and PSEN1 that have been shown to be associated with Alzheimer's disease. Both schizophrenia and Alzheimer's result in cognitive defects. Cognition is a complex mental process that integrates awareness, perception, reasoning, language, memory and judgment. Genes from our finding such as APBA2, PIN1, ITGA3, PAK7 and ABCA1 connect directly to genes associated with Alzheimer's. The APBA2 gene was identified in the full sample analysis and it has a role in the regulation of APP, the amyloid precursor protein. A copy number variation (CNV) at the APBA2 locus was recently found to be associated with schizophrenia. The PIN1 gene is an independent risk factor to SPG3A, a gene identified in the full sample analysis. PIN1 encodes an enzyme that have been shown to prevent the tangle-like lesions found in the brains of Alzheimer's disease patients, and it also plays a role in guarding against the development of amyloid peptide plaques. Genetic variations in the human PIN1 gene are associated with Alzheimer's disease. Reduced production of the Pin1 enzyme has been suggested to be of key importance in the onset of Alzheimer's disease. PIN1 promotes dephosphorylation of TAU, and regulates the cleavage of APP as well as amyloid beta production. ITGA3, identified from the full sample analysis, is located in a linkage schizophrenia candidate region. As part of the DAB1/RELN signaling pathway, this gene may contribute to appropriate neuronal placement in the developing cerebral cortex. This gene was also found to be epistatic to SPG3A. ITGA3 is predominantly expressed in brain, it promotes neurite outgrowth, and it may play a role in neurite development. The ABCA1 is an independent risk factor to NRG1. Located in close vicinity to the 9q linkage region associated with Alzheimer's. ABCA1 plays an important role in cellular cholesterol efflux, it has a potential in brain lipid transport and it regulates APP.

### Novel Pathway: Development and Synapse Formation

Schizophrenia appears to be a development disorder resulting when neurons form inappropriate connections during fetal development. This pathway includes genes from the full sample analysis such as WNT7A and NKD2 as well as genes from sub-analyses such as MSX1 and FZD7. All of them have a role in Wnt signaling. Wnt signaling is a canonical pathway that is active in the nervous system and that exhibits a dynamic pattern during forebrain development. The WNT7A gene encodes a protein that regulates axonal remodeling and synaptic differentiation in the cerebellum. The mouse and fly NKD2 homologs are dishevelled binding proteins acting as inducible antagonists of Wnt signals. It is therefore possible that genetic alteration of NKD2 leads to modulation of the WNT—beta-catenin signaling pathway. The MSX1 gene was found to be epistatic to the CIAS1 locus (a gene identified from the full sample analysis) and also in the female with age of onset over 25. MSX1 was reported to be implicated in the development and definition of the craniofacial skeleton and it is also known to be involved in limb, muscle and nail development. The FZD7 gene was identified as an independent risk factor to the SPG3A locus. FZD7 regulates Wnts and facilitates the Wnt signal cascade during embryonic mesoderm and neural induction. It is required for neural crest induction by Wnt in the developing vertebrate embryo.

### Novel Pathway: Long Term Potentiation

Several reports have suggested that schizophrenia is associated with disrupted plasticity in the cortex. It has been shown recently, that deficits in learning and memory in schizophrenia may be mediated through altered processes in long term potentiation (LTP). This pathway includes two genes that play an important role in LTP. The PTPRD gene corresponds to our top signal from the full sample analysis. PTPRD binds PTPRA, a gene that is considered as a novel member of the functional class of genes that control neuronal migration and synaptic plasticity. PTPRD is also involved in the regulation of synaptic plasticity or in the processes regulating learning and memory. Such gene is highly expressed in the developing mammalian nervous system, regulates neuroendocrine development, axonal regeneration and hippocampal LTP. In situ hybridization experiments to map Ptprd gene expression sites in the mouse embryo, postnatal stages and adulthood revealed that in the central nervous system, Ptprd expression starts at midgestation and lasts until adulthood. During CNS ontogeny, Ptprd mRNA distribution pattern changes from homogeneous to heterogeneous, long-lasting within specific centers highly labeled. Some of these centers are involved in stress control (hippocampal area CA2 and specific hypothalamic regions), and visual tract reticular thalamic nucleus, involved in the hallucination in schizophrenia, suggesting that Ptprd might have a role to play in these conditions. Finally, Ptprd mRNA in the nervous system is not limited to neuronal cells, since, the labeled oligodendrocyte that produce myelin sheaths around the bundles of axons were observed in the white matter regions, such as corpus callosum in the brain. Ptprd may, thus, be involved in the myelin production in the white matter.

The NRG2 gene relates through indirect interactions to PTPRA and plays an important role in neurodevelopment. Recent studies have shown that NRG2 is associated with schizophrenia. In pair-wise interaction tests, clear evidence of gene-gene interactions was detected for NRG1-NRG2, EGFR-NRG2, and suggestive evidence was also seen for ERBB4-NRG2.

### Neurodevelopment and Inflammation

Previously, the brain was considered as an immune privileged organ, not susceptible to inflammation or immune activation and was thought to be largely unaffected by systemic inflammatory and immune response processes. It is now accepted that the brain coordinates and regulates many aspects of the host defense response to several diseases including schizophrenia. Since many schizophrenia patients have autoimmune diseases, schizophrenia link to inflammation might help explain why many schizophrenic patients have co-morbid autoimmune diseases. The neurodevelopment and inflammation pathway is characterized by the presence of several genes that have been implicated in inflammation. Among them, genes such as interleukin-6 (IL-6) and interleukin-1β (IL-1β) have been shown to reduce significantly dendrite development and complexity of developing cortical neurons, consistent with the neuropathology of schizophrenia. IL-1β connects directly to NLRP3 and PAPP-A genes. NLRP3 was identified in the full sample analysis. This gene was found to be associated with various inflammatory diseases and also forms with other proteins, an inflammasome with high pro-ILB-processing activity. PAPP-A levels are elevated in acute coronary syndromes and are closely related to inflammation and oxidative stress. Also the PAPP-A expression is regulated by cytokines like IL-B1. Other genes in the pathway include BCAS1, VIPR2, RAD23B, TOM1 and CENPE. BCAS1 is a gene that binds to dynein and our preliminary expression analysis detected a brain-specific spliced variant. VIPR2 is a critical mediator of VIP neuroprotective properties against excitotoxic white matter lesions in the developing mouse brain. The protein encoded by RAD23B is a DNA repair enzyme but it has been shown to accumulate in neuronal inclusions in specific neurodegenerative disorders. Furthermore, RAD23B may play an important role in development since RAD23B (−/−) mice show impaired embryonic development. The TOM1 gene, epistatic to the WNT7A locus, was shown to be associated to bipolar disorder. Finally, in situ hybridization studies using mouse at different stages of development, revealed that KMO expression was also evident in the spleen and in the lymph nodes, emphasizing a potential role in the body immunosurveillance process.

### Schizophrenia and Drug Targets:

It has been suggested that the imbalance in the interrelated chemical reactions of the brain involving the neurotransmitters dopamine and glutamate (and possibly others) plays a role in schizophrenia.

The commonly prescribed drugs for schizophrenia are atypical antipsychotics. An important number of these antipsychotics were subjects to evaluation in a recent study, CATIE (Clinical Antipsychotic Trials of Intervention Effectiveness). Some issues such as insufficient efficacy and tolerability experienced by patients have been observed (74% of patients taking antipsychotics discontinued treatment within 18 months). Each medication has a specific mechanism of action, and many are meant to target a certain symptom or group of symptoms. Several approved FDA treatments have a mechanism of action that targets dopamine D2 receptor. In schizophrenic brain, it has been shown that the density of dopamine D2 receptor is high and its blockade is the main target for antipsychotic drugs.

Several compounds that target this receptor are already marketed; others are in clinical trials. DRD2 gene connects to AKT1, a gene that is present in schizophrenia GeneMap and that is in direct interaction with LIS1/PAFAH1B1, a gene discovered from the full sample analysis.

New treatments are developed and being tested. Several of them are targeting the N-methyl-D-aspartate receptors (NMDARs). Increasing evidence has suggested that the NMDAR hypofunction plays a key role in schizophrenia. Administration of noncompetitive NMDAR antagonists in humans and animals has been shown to produce behavioral symptoms that are remarkably similar to schizophrenia.

In the schizophrenia GeneMap, NMDAR connects directly to 3 of the identified genes. KMO and RASGRF2 are genes identified from the full sample analysis and NRG1 is a sub-phenotype gene.

The CHRNA7 gene is a nicotinic receptor subunit that is considered as an attractive target for novel therapeutic drugs for neuropsychiatric diseases. CHRNA7 interacts with the genes in the GeneMap such as APP, PSEN1 and MAPT. Both PSEN1 and APP interacts with APBA2 a gene identified from the full sample analysis. MAPT interacts with 2 genes in the GeneMap, both of these genes regulate MAPT activity. One of them is the PAK1 gene, epistasic with PTPRD and an independent risk factor to the SPG3A locus. The other gene is PIN1, an independent risk factor to the SPG3A locus.

Other drugs targeting glutamate receptor subunits GRM2, GRM3, or GRM5 are currently in clinical trials. GRM2 is in direct interaction with GRIP1, a gene in epistasis with PTPRD. GRM3 and GRM5 interact with subunits of NMDAR and ERBB4, two genes in the GeneMap. Drugs targeting subunits of the serotonin receptor, 5-HT1 and 5-HT2, are already on the market whereas others are clinical trials. Serotonin receptor subunits directly interact with genes in the GeneMap. 5-HT1 connects to NMDAR and Calmodulin and 5-HT2 connects to Calmodulin, DLG3 and DLG4.

1.-20. (canceled)

21. A method of diagnosing schizophrenia, the predisposition to schizophrenia, or the progression or prognostication of schizophrenia, comprising determining the amount and/or concentration of at least one polypeptide from Tables 2-4 and/or at least one nucleic acid encoding the polypeptide present in said biological sample.

22. The method of claim 21, wherein the diagnosing comprises the steps of: (a) obtaining a biological sample of mammalian body fluid or tissue to be diagnosed; (b) comparing the amount and/or concentration of said polypeptide and/or nucleic acid encoding the polypeptide determine in said biological sample with the amount and/or concentration of said polypeptide and/or nucleic acid encoding the polypeptide as determined in a control sample, wherein the difference in the amount of said polypeptide and/or nucleic acid encoding the polypeptide is indicative of schizophrenia or the stage of schizophrenia.

23. The method of claim 21, wherein a nucleic acid probe is used for determining the amount and/or concentration of at least one nucleic acid sequence from Tables 2-4 encoding the polypeptide.

24. The method of claim 23, wherein said nucleic acid probe is selected from the nucleic acid sequences designated as SEQ ID NO: 1 to 19625.

25. The method of claim 23, wherein said nucleic acid probe comprises nucleic acids hybridizing to the nucleic acid sequences designated as SEQ ID NO: 1 to 19625, and/or fragments thereof.

26. The method of claim 23, wherein said nucleic acid probe comprises nucleic acids hybridizing to at least five nucleic acid sequences from Table 2, 3 or 46.

27. The method of claim 23, wherein said nucleic acid probe specifically hybridizes to at least 10 nucleic acid sequences from Tables 2-4.

28.-31. (canceled)

32. The method of claim 23, wherein said nucleic acid probe is at least about 10 nucleotides in length.

33.-34. (canceled)

35. The method of claim 23, wherein a PCR technique is used for determining the amount and/or concentration of at least one nucleic acid from Tables 2-4.

36. The method of claim 21, wherein a specific antibody is used for determining the amount and/or concentration of at least one polypeptide from Tables 2-4.

37.-38. (canceled)

39. A method of detecting susceptibility to schizophrenia comprising detecting at least one mutation or polymorphism in the nucleic acid molecule selected from Tables 2-4 in a patient.

40. The method of claim 39, wherein said method comprises hybridizing a probe to said patient's sample of DNA or RNA under stringent conditions which allow hybridization of said probe to nucleic acid comprising said mutation or polymorphism, wherein the presence of a hybridization signal indicates the presence of said mutation or polymorphism in at least one gene from Tables 2-4.

41.-42. (canceled)

43. The method of claim 39, wherein said method comprises sequencing at least one gene from Tables 2-4 in a sample of RNA or DNA from a patient.

44.-48. (canceled)

49. The method of claim 39, wherein the mutation is selected from the group consisting of at least one of the SNPs from Tables 5.1, 6.1, 7.1, 8.1, 9.2, 10.1, 11.1, 12.1, 13.1, 14.1, 15.1, 16.2, 17.2, 18.2, 19.2, 20.2, 21.1, 22.1, 23.1, 24.1, 25.1, 26.1, 27.1, 28.1, 29.1, 30.1, 31.1, 32.2, 33.1, 34.1 and 35.1, alone or in combination.

50. The method of claim 21, further comprising comparing the level of expression or activity of a polypeptide of Tables 2-4 in a test sample from a patient with the level of expression or activity of the same polypeptide in a control sample wherein a difference in the level of expression or activity between the test sample and control sample is indicative of SCHIZOPHRENIA disease.

51. (canceled)

52. A method of diagnosing susceptibility to schizophrenia in an individual, comprising screening for an at-risk haplotype of at least one gene or gene region from Tables 2-4, that is more frequently present in an individual susceptible to schizophrenia compared to a control individual, wherein the presence of the at-risk haplotype is indicative of a susceptibility to schizophrenia.

53. The method of claim 52 wherein the at-risk haplotype is indicative of increased risk for schizophrenia.

54. The method of claim 53, wherein the risk is increased at least about 20%.

55. The method of claim 52, wherein the at-risk haplotype is characterized by the presence of at least one single nucleotide polymorphism from Tables 5.1, 6.1, 7.1, 8.1, 9.2, 10.1, 11.1, 12.1, 13.1, 14.1, 15.1, 16.2, 17.2, 18.2, 19.2, 20.2, 21.1, 22.1, 23.1, 24.1, 25.1, 26.1, 27.1, 28.1, 29.1, 30.1, 31.1, 32.2, 33.1, 34.1 and 35.1.

56.-59. (canceled)

60. The method of claim 59, wherein determining the presence of an at-risk haplotype is performed by electrophoretic analysis, restriction length polymorphism analysis, sequence analysis or hybridization analysis.

61.-63. (canceled)

64. A method of diagnosing a susceptibility to schizophrenia, comprising detecting an alteration in the expression or composition of a polypeptide encoded by at least one gene from Tables 2-4 in a test sample, in comparison with the expression or composition of a polypeptide encoded by said gene in a control sample, wherein the presence of an alteration in expression or composition of the polypeptide in the test sample is indicative of a susceptibility to schizophrenia.

65. The method of claim 64, wherein the alteration in the expression or composition of a polypeptide encoded by said gene comprises expression of a splicing variant polypeptide in a test sample that differs from a splicing variant polypeptide expressed in a control sample.

66. A drug screening assay comprising: (a) administering a test compound to an animal having schizophrenia, or a cell population isolated therefrom; and (b) comparing the level of gene expression of at least one gene from Tables 2-4 in the presence of the test compound with the level of said gene expression in normal cells; wherein test compounds which provide the level of expression of one or more genes from Tables 2-4 similar to that of the normal cells are candidates for drugs to treat SCHIZOPHRENIA disease.

67.-68. (canceled)

69. A method for predicting the efficacy of a drug for treating schizophrenia in a human patient, comprising: (a) obtaining a sample of cells from the patient; (b) obtaining a gene expression profile from the sample in the absence and presence of the drug; the gene expression profile comprising one or more genes from Tables 2-4; and (c) comparing the gene expression profile of the sample with a reference gene expression profile, wherein similarity between the sample expression profile and the reference expression profile predicts the efficacy of the drug for treating schizophrenia in the patient.

70. The method of claim 69, further comprising exposing the sample to the drug for treating schizophrenia prior to obtaining the gene expression profile of the sample.

71.-73. (canceled)

74. The method of claim 69, wherein the gene expression profile comprises expression values for all of the genes listed in Tables 2-4.

75.-76. (canceled)

77. The method of claim 76, wherein the oligonucleotides comprises nucleic acid molecules at least 95% identical to the gene sequences from Tables 2-4.

78. The method of claim 69, wherein the reference expression profile is that of cells derived from patients that do not have schizophrenia.

79.-80. (canceled)

81. A method for predicting the efficacy of a drug for treating schizophrenia in a human patient, comprising: (a) obtaining a sample of cells from the patient; (b) obtaining a set of genotypes from the sample, wherein the set of genotypes comprises genotypes of one or more polymorphic loci from Tables 2-35; and (c) comparing the set of genotypes of the sample with a set of genotypes associated with efficacy of the drug, wherein similarity between the set of genotypes of the sample and the set of genotypes associated with efficacy of the drug predicts the efficacy of the drug for treating schizophrenia in the patient.

82.-84. (canceled)

85. The method of claim 81, wherein the set of genotypes from the sample comprises genotypes of at least two of the polymorphic loci listed in Tables 2-35.

86. The method of claim 81 wherein the set of genotypes from the sample is obtained by hybridization to allele-specific oligonucleotides complementary to the polymorphic loci from Tables 2-35, wherein said allele-specific oligonucleotides are contained on a microarray.

87. The method of claim 86, wherein the oligonucleotides comprise nucleic acid molecules at least 95% identical to SEQ ID from Tables 2-35.

88.-117. (canceled)

118. A method for identifying a gene that regulates drug response in schizophrenia, comprising: (a) obtaining a gene expression profile for at least one gene from Tables 2-4 in a resident tissue cell induced for a pro-inflammatory like state in the presence of the candidate drug; and (b) comparing the expression profile of said gene to a reference expression profile for said gene in a cell induced for the pro-inflammatory like state in the absence of the candidate drug, wherein genes whose expression relative to the reference expression profile is altered by the drug may identifies the gene as a gene that regulates drug response in schizophrenia.

119.-121. (canceled)

122. A kit for assessing a patient's risk of having or developing schizophrenia, comprising:
(a) means for detecting:
the differential expression, relative to a normal cell, of at least one gene shown in Tables 2-4 or the gene product thereof;
a sequence of at least one gene in Tables 2-4; or
a genotype of at least one polymorphic locus shown in Tables 2-35; and
(b) instructions for correlating the differential expression or the presence of said gene or gene product or the presence of the genotype with a patient's risk of having or developing schizophrenia. [Support in original claims 124 and 126]

123. The kit of claim 122, wherein the means includes nucleic acid probes for detecting the level of mRNA of said genes.

124. (canceled)

125. The kit of claim 124, wherein the means includes an immunoassay for detecting the level of at least one gene product from Tables 2-4.

126. (canceled)

127. The kit of claim 126, wherein the means includes nucleic acid probes for detecting the genotype of said at least one polymorphic locus.

128.-136. (canceled)

137. A method of assessing a patient's risk of having or developing SCHIZOPHRENIA disease, comprising: (a) determining the level of expression of at least one gene from Tables 2-4 or gene products thereof, and comparing the level of expression to a normal cell; and (b) assessing a patient's risk of having or developing SCHIZOPHRENIA disease by determining the correlation between the differential expression of said genes or gene products with known changes in expression of said genes measured in at least one patent suffering from SCHIZOPHRENIA disease.

138. A method of assessing a patient's risk of having or developing schizophrenia, comprising (a) determining a genotype for at least one polymorphic locus from Tables 2-35 in a patient; (b) comparing said genotype of (a) to a genotype for at least one polymorphic locus from Tables 2-35 that is associated with schizophrenia; and (c) assessing the patient's risk of having or developing schizophrenia, wherein said patient has a higher risk of having or developing schizophrenia if the genotype for at least one polymorphic locus from Tables 2-35 in said patient is the same as said genotype for at least one polymorphic locus from Tables 2-35 that is associated with schizophrenia.

139.-140. (canceled)
